(19) United States
(12) Patent Application Publication
(10) Pub. No.: US 2026/0248926 A1
(43) Pub. Date: Aug. 27, 2026

(54) BIFUNCTIONAL COMPOUND AND PHARMACEUTICAL COMPOSITION COMPRISING THE BIFUNCTIONAL COMPOUND, AND METHOD FOR TREATING ANDROGEN RECEPTOR RELATED DISEASE BY USING THE SAME

(71) Applicant: ANHORN MEDICINES CO., LTD., Taipei City (TW)

(72) Inventors: Chu-Chiang Lin, Taipei City (TW); Hung-Chuan Chen, New Taipei City (TW); Pei-Chin Cheng, Changhua County (TW); Chih-Chang Chou, Taoyuan City (TW)

(21) Appl. No.: 18/879,348

(22) PCT Filed: Jun. 29, 2023
(86) PCT No.: PCT/CN2023/103568

§ 371 Date: Dec. 27, 2024

Related U.S. Application Data
(60) Provisional application No. 63/357,054, filed on Jun. 30, 2022.

(51) Int. Cl. A61K 47/55; A61K 47/54

(52) U.S. Cl. CPC A61K 47/55; A61K 47/545

(57) ABSTRACT

Provided is a bifunctional compound, or a pharmaceutically acceptable salt, hydrate, solvate, metabolite or prodrug thereof, wherein the bifunctional compound is represented by Formula (I):

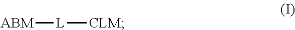

wherein:
ABM is an androgen receptor binding moiety;
-L- is a linking moiety; and
CLM is a cereblon E3 ubiquitin ligase binding moiety represented by Formula (II)-1:

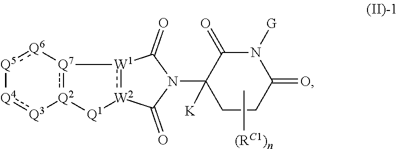

wherein one end of the -L- is covalently joined to Q₃, Q₄, Q₅ or Q₆; and the other end of the -L- is covalently joined to the ABM. Also provided are a pharmaceutical composition comprising the bifunctional compound and a method for treating an androgen receptor related disease by administering the bifunctional compound.

### BACKGROUND OF THE INVENTION

### 1. Field of the Invention

The present disclosure relates to a bifunctional compound; and a pharmaceutical composition comprising the bifunctional compound and a method for treating an androgen receptor related disease by administering the bifunctional compound.

### 2. Description of the Prior Arts

Protein-protein interactions are difficult to be targeted using small molecules because proteins have large contact surfaces and the shallow grooves or flat interfaces thereon may get involved in the interactions. On the other hand, tagging the pathogenic protein with ubiquitin and the eventual degradation of the pathogenic protein by the 26S proteasome system has demonstrated that this modality can provide an extended and thorough removal of the cause of disease (Sun et al., Signal Transduct. Target Ther. 2019, 4:64). In addition to the E1 and E2 enzymes, E3 ubiquitin ligases (also known as E3 ligases) and their substrate recognition proteins confer the substrate specificity for ubiquitination. They are the critical components for specific and selective degradation of target protein substrates. Recent development of targeted protein degradation indicated E3 ligases such as cereblon (CRBN) E3 ligase, von Hippel-Lindau disease tumor suppressor (VHL) E3 ligase, mouse double minute 2 protein (MDM2) E3 ligase, and cell inhibitor of apoptosis protein (cIAP) E3 ligase have been utilized successfully for small molecule protein degrader design. These molecules are likely to become therapeutic candidates (Wang et al., Acta Pharm Sin B. 2020 February; 10 (2): 207-238).

One E3 ligase with therapeutic potential is cereblon E3 ligase, a protein in humans that is encoded by the CRBN gene. CRBN orthologs are highly conserved from plants to humans. Cereblon forms an E3 ubiquitin ligase complex with damaged DNA binding protein 1 (DDB1), Cullin-4A (CUL4A), and Regulator of Cullins 1 (ROC 1). This complex ubiquitinates a number of other proteins (Vriend et al., Front Mol Biosci. 2018, 5:19).

Androgen receptor (AR) belongs to a nuclear hormone receptor family that is activated by androgens, such as testosterone and dihydrotestosterone. Upon binding of androgen, AR is translocated into the nucleus where it acts as a transcription factor to promote gene expression responsible for male sexual characteristics. While AR is responsible for the development of male sexual characteristics, it also drives the growth and survival of prostate cancer cells (Salami et al., Commun. Biol. 2018, 1:100).

AR signaling suppression is a common strategy for treating prostate cancer. Prostate cancer is the second most diagnosed cancer and the fifth leading cause of death in men worldwide. The 5-year survival rate for most men with local or regional prostate cancer is nearly 100%. For men diagnosed with prostate cancer that has spread to other parts of the body, the 5-year survival rate is 31%. Based on GLOBOCAN estimates, 1.41 million new cases of prostate cancer were reported in 2020 with 375 thousand deaths worldwide. For decades, the androgen deprivation therapy (ADT), by either surgical or chemical castration, has been the standard treatment for prostate cancer management. However, a castration-resistant form of prostate cancer eventually develops, whereby tumor cell proliferation resumes despite sub-castration levels of serum testosterone. With the approval of second-generation antiandrogen drugs such as abiraterone acetate (ABI), enzalutamide (ENZ), and recently approved apalutamide (APA) and darolutamide (DARO), overall survival rate of castration-resistant prostate cancer (CRPC) patients was improved. But, as frequently seen in cancer chemotherapy, drug resistant escape mutations eventually appear while the disease progresses (Zhao et al., Mol Cancer Ther. 2020, 19 (8): 1708-1718). In addition, it is also found that androgen and AR involve in many skin conditions, such as androgenetic alopecia, acne, hirsutism, atopic dermatitis, and the like. In the skin, significant amounts of sexual hormones are synthesized, and androgen affects hair growth, epidermal barrier homeostasis, wound healing, sebaceous gland growth, differentiation and so on. Nowadays, antiandrogen drugs such as clascoterone, cyproterone acetate and flutamide have been used for treating these skin conditions. The level of androgen and AR may play important roles in the development of the diseases involving AR signaling. (Zhou et al., Journal of Biosciences and Medicines 2022, 10:180-200).

The present disclosure provides an alternative approach via proteolysis targeting chimeric (PROTAC) bifunctional compounds for the treatment of AR-mediated or AR-dependent diseases or disorders. Related PROTAC patent applications are disclosed in WO2018071606, WO2018144649 and WO2021143816. However, compounds having better effects for treating androgen receptor related diseases or disorders are still needed.

### SUMMARY OF THE INVENTION

The present disclosure provides proteolysis targeting chimeric (PROTAC) bifunctional compounds comprising both a cereblon (CRBN) E3 ubiquitin ligase binding moiety and an androgen receptor binding moiety, which redirect a ubiquitin proteasome degradation system to degrade androgen receptors, and degrade the androgen receptors and/or otherwise inhibit the androgen receptors. Unexpectedly, these bifunctional compounds demonstrate higher potency in the degradation of androgen receptors.

In one aspect, the present disclosure provides a bifunctional compound, or a pharmaceutically acceptable salt, hydrate, solvate, metabolite or prodrug thereof, wherein the bifunctional compound is represented by Formula (I):

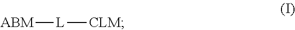

wherein:
ABM is an androgen receptor binding moiety;
-L- is a linking moiety; and
CLM is a cereblon E3 ubiquitin ligase binding moiety represented by Formula (II)-1:

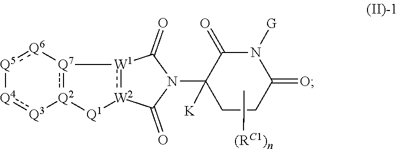

wherein
[symbol] represents a single bond or a double bond;
one end of the -L- is covalently joined to Q₃, Q₄, Q₅ or Q₆; and the other end of the -L- is covalently joined to the ABM;
W¹ and W² are each independently CR^{C2} or N when a single bond is present between W¹ and W²; or, W¹ and W² are each C when a double bond is present between W¹ and W²;
G is selected from the group consisting of —H, —OH, —CH₂OH, —R^{C3}OC(═O)OR^{C4}, —R^{C3}OC(═O)NR^{C4}R^{C5}, and 2-(trimethylsilyl) ethoxymethyl group;
Q¹ is O, S or NR^{C6};
Q² and Q⁷ are each independently N or CR^{C2} when a single bond is present between Q² and Q⁷; or, Q² and Q⁷ are each C when a double bond is present between Q² and Q⁷;
when the one end of the -L- is covalently joined to any one atom selected from Q₃, Q₄, Q₅ and Q₆; the atom covalently joined with the -L- is CR^{C2} when two single bonds are each independently present between the C atom of CR^{C2} and its two adjacent atoms on the ring, or the atom covalently joined with the -L- is C when a double bond is present between the C atom and one of its two adjacent atoms on the ring; and, the other atoms selected from Q³, Q⁴, Q⁵ and Q⁶ which are not covalently joined with the -L- are each independently O, S, C(R^{C2})₂ or NR^{C2} when two single bonds are each independently present between the O atom, S atom, C atom of C(R^{C2})₂ or N atom of NR^{C2} and its two adjacent atoms on the ring, or the other atoms selected from Q³, Q⁴, Q⁵ and Q⁶ which are not covalently joined with the -L- are each independently CR^{C2} when a double bond is present between the C atom of CR^{C2} and one of its two adjacent atoms on the ring;
K is selected from the group consisting of —H, an unsubstituted alkyl group, an alkyl group substituted by R^{C7}, an unsubstituted cycloalkyl group, and a cycloalkyl group substituted by R^{C7}; K is bound to the 6-membered ring with a stereospecific bond or a non-stereospecific bond.
R^{C1} is selected from the group consisting of an unsubstituted alkyl group, an alkyl group substituted by R^{C8}, an unsubstituted aryl group, an aryl group substituted by R^{C8}, an unsubstituted alkyl-aryl group, an alkyl-aryl group substituted by R^{C8}, an unsubstituted alkoxyl group, and an alkoxyl group substituted by R^{C8},
R^{C2} is selected from the group consisting of —H, -D, a halo group, —CH₂OH, —NR^{C4}R^{C5}, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, and an aryl group substituted by one or more halo groups;
R^{C3} is selected from the group consisting of an unsubstituted alkylene group, and an alkylene group substituted by R^{C7};
R^{C4} and R^{C5} are each independently selected from the group consisting of —H, an unsubstituted alkyl group, an alkyl group substituted by R^{C9}, an unsubstituted cycloalkyl group, a cycloalkyl group substituted by R^{C9}, an unsubstituted heterocyclyl group, a heterocyclyl group substituted by R^{C9}, an unsubstituted aryl group, an aryl group substituted by R^{C9}, an unsubstituted heteroaryl group, and a heteroaryl group substituted by R^{C9};
R^{C6} is selected from the group consisting of —H, a halo group, —CH₂OH, 2-(trimethylsilyl) ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C7} is selected from the group consisting of a halo group, —CH₂OH, —NR^{C4}R^{C5}, 2-(trimethylsilyl) ethoxymethyl, an alkoxyl group, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C8} is selected from the group consisting of a halo group, —CH₂OH, —NR^{C4}R^{C5}, 2-(trimethylsilyl) ethoxymethyl, an unsubstituted cycloalkyl group, a cycloalkyl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C9} is selected from the group consisting of a halo group, —CH₂OH, 2-(trimethylsilyl) ethoxymethyl, and an alkoxyl group;
n is 0, 1, 2, 3 or 4; and
the -L- is represented by Formula (III):

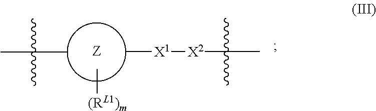

wherein
Z is selected from the group consisting of a 3- to 8-membered monocyclic ring, a 5- to 12-membered bicyclic ring, a 8- to 15-membered tricyclic ring and a 6- to 12-membered spiro bicyclic ring, each independently having 0 to 4 heteroatoms;
R^{L1} is selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, an unsubstituted C₁₋₆ alkoxyl group, a keto group, and an oxide group;
X¹ is a methylene group or an ethylene group, each of which is unsubstituted or substituted by alkyl or cycloalkyl;
X² is selected from the group consisting of a 5- to 8-membered arylene group, a 5- to 8-membered heteroarylene group having 1 to 3 heteroatoms, a 3- to 7-membered cyclic alkylene group, a 3- to 7-membered heterocyclic alkylene group having 1 to 2 heteroatoms, a 3- to 8-membered cyclic alkenylene group, a 3- to 8-membered cyclic heteroalkenylene group having 1 to 3 heteroatoms, and a 6- to 12-membered spiro bicyclic bivalent group having 0 to 4 heteroatoms, each of which is unsubstituted or substituted by alkyl or cycloalkyl;
m is 0, 1, 2, 3, 4, 5 or 6; v1 is 1 or 2; and v2 is 1 or 2; and
the heteroatom is selected from N, O and S.

In another aspect, the present disclosure provides a pharmaceutical composition comprising an effective amount of the above-mentioned compound, or the pharmaceutically acceptable salt, hydrate, solvate, metabolite or prodrug thereof; and a pharmaceutically acceptable carrier.

In yet another aspect, the present disclosure provides a method for treating an androgen receptor related disease in a subject in need thereof, comprising administering an effective amount of the above-mentioned compound, or the pharmaceutically acceptable salt, hydrate, solvate, metabolite or prodrug thereof, or the above-mentioned pharmaceutical composition to the subject.

In some embodiments, the CLM is represented by Formula (II)-2:

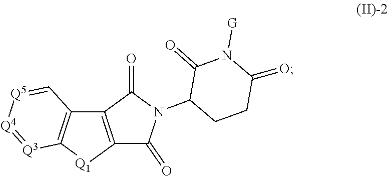

wherein
one end of the -L- is covalently joined to Q₃, Q₄ or Q₅;
G is selected from the group consisting of —H, —OH, and —CH₂OH;
Q¹ is O, S or NR^{C6};
when the one end of the -L- is covalently joined to any one atom selected from Q₃, Q₄ and Q₅; the atom attached with the -L- is C; and, the other atoms selected from Q³, Q⁴ and Q⁵ which are not attached with the -L- are each independently CR^{C2}; and
R^{C2} is selected from the group consisting of —H, -D, a halo group, an unsubstituted alkyl group, and an alkyl group substituted by one or more halo groups;
R^{C6} is selected from the group consisting of —H, —CH₂OH, an unsubstituted C₁₋₆ alkyl group, a C₁₋₆ alkyl group substituted by one or more halo groups.

In some embodiments, the heteroatoms in Formula (II)-1 are each independently selected from N, O and S.

In some embodiments, G is selected from the group consisting of —H, —OH, —CH₂OH, —CH₂OCOOCH₃ and 2-(trimethylsilyl) ethoxymethyl group.

In some embodiments, the 2-(trimethylsilyl) ethoxymethyl group is also abbreviated as an SEM group.

In some embodiments, K is bound to the 6-membered ring with a stereospecific bond:

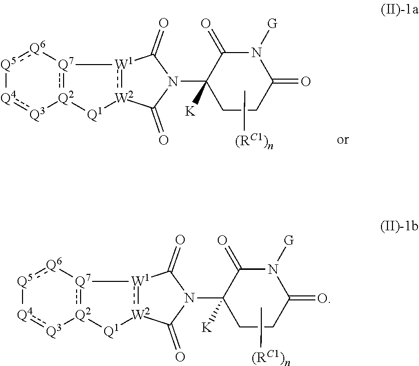

In the present invention, the carbon on the 6-membered ring which is attached with K is a chiral carbon center, and the bifunctional compound, or a pharmaceutically acceptable salt, hydrate, solvate, metabolite or prodrug thereof may exist two stereoisomers having a CLM represented by Formula (II)-la and Formula (II)-1b. The bifunctional compound, or a pharmaceutically acceptable salt, hydrate, solvate, metabolite or prodrug thereof may have one above-mentioned stereoisomer or both stereoisomers.

In some embodiments, K is selected from the group consisting of —H, an unsubstituted C₁₋₆ alkyl group, a C₁₋₆ alkyl group substituted by R^{C7}, and a C₃₋₈ cycloalkyl group. In some embodiments, K may be an unsubstituted C₁₋₃ alkyl group, or a C₁₋₃ alkyl group substituted by R^{C7}.

In some embodiments, K is an alkyl selected from the group consisting of a linear alkyl and a branched alkyl, each of which is unsubstituted or substituted by R^{C7}. In some embodiments, K is an alkyl selected from the group consisting of a linear C₁₋₆ alkyl and a branched C₁₋₆ alkyl, each of which is unsubstituted or substituted by R^{C7}. In some embodiments, K is an alkyl selected from the group consisting of a linear C₁₋₃ alkyl and a branched C₁₋₃ alkyl, each of which is unsubstituted or substituted by R^{C7}.

In some embodiments, R^{C1} may be an unsubstituted C₁₋₆ alkyl group, a C₁₋₆ alkyl group substituted by R^{C8}, an unsubstituted C₃₋₈ aryl group, a C₃₋₈ aryl group substituted by R^{C8}, an unsubstituted C₃₋₈ alkyl-aryl group, a C₃₋₈ alkyl-aryl group substituted by R^{C8}, an unsubstituted C₁₋₆ alkoxyl group, or a C₁₋₆ alkoxyl group substituted by R^{C8}. In some embodiments, R^{C1} may be an unsubstituted C₁₋₃ alkyl group, a C₁₋₃ alkyl group substituted by R^{C8}, an unsubstituted C₁₋₃ alkoxyl group, or a C₁₋₃ alkoxyl group substituted by R^{C8}.

In some embodiments, R^{C1} is an alkyl selected from the group consisting of a linear alkyl and a branched alkyl, each of which is unsubstituted or substituted by R^{C8}. In some embodiments, R^{C1} is an alkyl selected from the group consisting of a linear C₁₋₆ alkyl and a branched C₁₋₆ alkyl, each of which is unsubstituted or substituted by R^{C8}. In some embodiments, R^{C1} is an alkyl selected from the group consisting of a linear C₁₋₃ alkyl and a branched C₁₋₃ alkyl, each of which is unsubstituted or substituted by R^{C8}.

In the present disclosure, a halo group may be F, Cl, Br or I. In the present disclosure, a halo group may be F or Cl. In the present disclosure, a halo group is F.

In the present disclosure, R^{C2} is selected from the group consisting of —H, -D (deuterium), a halo group, an unsubstituted C₁₋₆ alkyl group, and a C₁₋₆ alkyl group substituted by one or more halo groups. In the present disclosure, R^{C2} is selected from the group consisting of —H, -D, —F, —Cl, an unsubstituted C₁₋₃ alkyl group, and a C₁₋₃ alkyl group substituted by one or more halo groups.

In the present disclosure, R^{C4} and R^{C5} are each independently selected from the group consisting of an unsubstituted C₁₋₆ alkyl group, a C₁₋₆ alkyl group substituted by R^{C9}, an unsubstituted C₃-s cycloalkyl group, a C₃₋₈ cycloalkyl group substituted by R^{C9}, an unsubstituted C₃₋₈ heterocyclyl group, a C₃₋₈ heterocyclyl group substituted by R^{C9}, a C₃₋₈ aryl group, and a C₃₋₈ heteroaryl group. In the present disclosure, R^{C4} and R^{C5} are each independently selected from the group consisting of an unsubstituted C₁₋₃ alkyl group, and a C₁₋₃ alkyl group substituted by R^{C9}. In the present disclosure, R^{C4} may be methyl, ethyl n-propyl or isopropyl.

In some embodiments, R^{C4} and R^{C5} are each independently an alkyl selected from the group consisting of a linear alkyl and a branched alkyl, each of which is unsubstituted or substituted by R^{C9}. In some embodiments, R^{C4} and R^{C5} are each independently an alkyl selected from the group consisting of a linear C₁₋₆ alkyl and a branched C₁₋₆ alkyl, each of which is unsubstituted or substituted by R^{C9}. In some embodiments, R^{C4} and R^{C5} are each independently an alkyl selected from the group consisting of a linear C₁₋₃ alkyl and a branched C₁₋₃ alkyl, each of which is unsubstituted or substituted by R^{C9}.

In some embodiments, the CLM is represented by Formula (II)-1 or Formula (II)-2, wherein Q¹ is NR^{C6}; and R^{C6} is —H, an unsubstituted C₁₋₆ alkyl group, or a C₁₋₆ alkyl group substituted by one or more halo groups. In some embodiments, Q¹ is NR^{C6}; and R^{C6} is H, an unsubstituted C₁₋₃ alkyl group, or a C₁₋₃ alkyl group substituted by one or more halo groups. In some embodiments, Q¹ is NR^{C6}; and R^{C6} is an unsubstituted C₁₋₃ alkyl group selected from methyl, ethyl, n-propyl, and isopropyl. In some embodiments, Q¹ is NR^{C6}, and R^{C6} is a C₁₋₃ alkyl group selected from methyl, ethyl, n-propyl, isopropyl, which is substituted by one or more halo groups independently selected from F, Cl and Br.

In some embodiments, one end of the -L- is covalently joined to Q₄ or Q₅. In some embodiments, one end of the -L- is covalently joined to Q₄.

In the three-ring system (formed by one N atom, two C atoms, W¹, W², Q¹, Q², Q³, Q¹, Q⁵, Q⁶ and Q⁷) in the Formula (II)-1 or Formula (II)-2, both double bond and single bond are represented or understood within the context of the bifunctional compound shown and well-known rules for valence interactions.

In some embodiments, the -L- is represented by Formula (III):

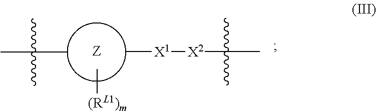

wherein
Z is selected from the group consisting of a 3- to 8-membered monocyclic ring, a 6- to 10-membered bicyclic ring and a 8- to 10-membered spiro bicyclic ring, each independently having 1 to 2 heteroatoms;
X¹ is an unsubstituted methylene group, or a methylene group substituted by alkyl or cycloalkyl;
X² is selected from the group consisting of a 3- to 7-membered heterocyclic alkylene group having 1 to 2 heteroatoms, a 3- to 8-membered cyclic heteroalkenylene group having 1 to 2 heteroatoms, and a 6- to 12-membered spiro bicyclic bivalent group having 1 to 2 heteroatoms, each of which is unsubstituted or substituted by alkyl or cycloalkyl;
m is 0, 1 or 2; v1 is 1 or 2; and v2 is 1.

In some embodiments, the heteroatoms in Formula (III) are each independently selected from N, O and S. In some embodiments, the Z ring comprises one or two heteroatoms selected from N, O and S.

In some embodiments, Z is an unsubstituted 3-, 4-, 5-, 6- or 7-membered heterocyclic alkylene group having 1 or 2 heteroatoms. In some embodiments, Z is a 6-, 7-, 8-, 9-, 10-, 11- or 12-membered spiro bicyclic bivalent group having 1 or 2 heteroatoms.

In some embodiments, R^{L1} is selected from the group consisting of an unsubstituted C₁₋₃ alkyl group, a C₁₋₃ alkyl group substituted by a C₁₋₃ alkoxyl group, a C₁₋₃ alkyl group substituted by one or more halo groups, a halo group, a C₁₋₃ alkoxyl group, a keto group, or an oxide group. In some embodiments, R^{L1} is an oxide group which is attached to one heteroatom N on the Z ring to form an N-oxide group (N⁺—O⁻).

In some embodiments, R^{L1} is an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, R^{L1} is an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups.

In some embodiments, X¹ is a methylene group substituted by 1 or 2 C₁₋₆ alkyl. In some embodiments, X¹ is a methylene group substituted by 1 or 2 C₁₋₃ alkyl. In some embodiments, X¹ is a methylene group substituted by 1 or 2 C₃₋₆ cycloalkyl. In some embodiments, X¹ is a methylene group substituted by 1 or 2 C₃₋₆ cycloalkyl.

In some embodiments, X¹ is an ethylene group substituted by 1 to 4 C₁₋₆ alkyl. In some embodiments, X¹ is an ethylene group substituted by 1 to 4 C₁₋₃ alkyl. In some embodiments, X¹ is an ethylene group substituted by 1 to 4 C₃₋₆ cycloalkyl. In some embodiments, X¹ is an ethylene group substituted by 1 to 4 C₃₋₆ cycloalkyl.

In some embodiments, X² is a 3-, 4-, 5-, 6- or 7-membered heterocyclic alkylene group having 1 or 2 heteroatoms, each of which is unsubstituted or substituted by alkyl or cycloalkyl. In some embodiments, X² is a 3-, 4-, 5-, 6-, 7- or 8-membered cyclic heteroalkenylene group having 1 or 2 heteroatoms, each of which is unsubstituted or substituted by alkyl or cycloalkyl. In some embodiments, X² is a 6-, 7-, 8-, 9-, 10-, 11- or 12-membered spiro bicyclic bivalent group having 1 or 2 heteroatoms, each of which is unsubstituted or substituted by alkyl or cycloalkyl.

In some embodiments, X² is a 3-membered heterocyclic alkylene group with 1 heteroatom and substituted by 1 to 3 alkyl or cycloalkyl, a 3-membered heterocyclic alkylene group with 2 heteroatoms and substituted by 1 to 2 alkyl or cycloalkyl, a 4-membered heterocyclic alkylene group with 1 heteroatom and substituted by 1 to 5 alkyl or cycloalkyl, a 4-membered heterocyclic alkylene group with 2 heteroatoms and substituted by 1 to 4 alkyl or cycloalkyl, a 5- to 7-membered heterocyclic alkylene group with 1 to 2 hetero atoms and substituted by 1 to 6 alkyl or cycloalkyl.

In some embodiments, X² is a 3-membered cyclic heteroalkenylene group having 1 heteroatom and substituted by 1 to 3 alkyl or cycloalkyl, a 4-membered cyclic heteroalkenylene group having 1 heteroatom and substituted by 1 to 3 alkyl or cycloalkyl, a 4-membered cyclic heteroalkenylene group having 2 heteroatoms and substituted by 1 to 2 alkyl or cycloalkyl, a 5-membered cyclic heteroalkenylene group having 1 heteroatom and substituted by 1 to 5 alkyl or cycloalkyl, a 5-membered cyclic heteroalkenylene group having 2 heteroatoms and substituted by 1 to 4 alkyl or cycloalkyl, a 6-, 7- or 8-membered cyclic heteroalkenylene group having 1 heteroatom and substituted by 1 to 6 alkyl or cycloalkyl, a 6-, 7- or 8-membered cyclic heteroalkenylene group having 2 heteroatoms and substituted by 1 to 6 alkyl or cycloalkyl.

In some embodiments, the -L- is selected from the group consisting of

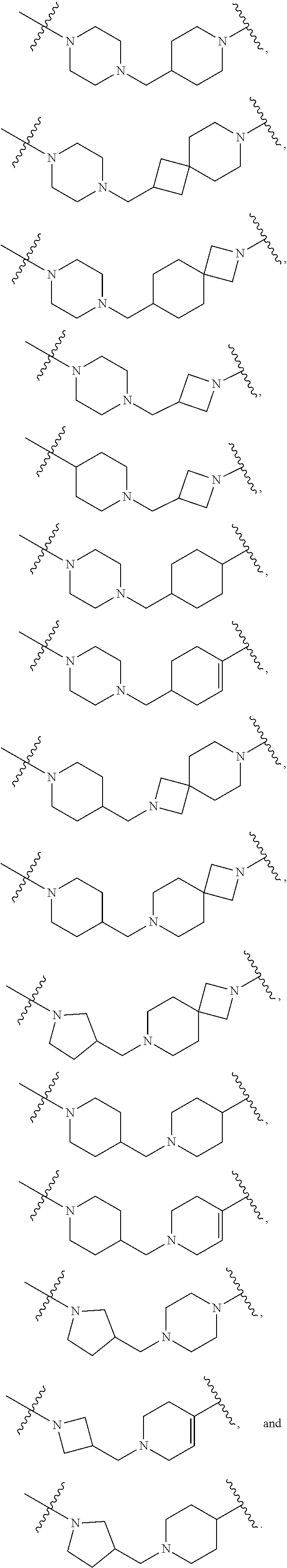

In some embodiments, the ABM is an androgen receptor binding moiety represented by

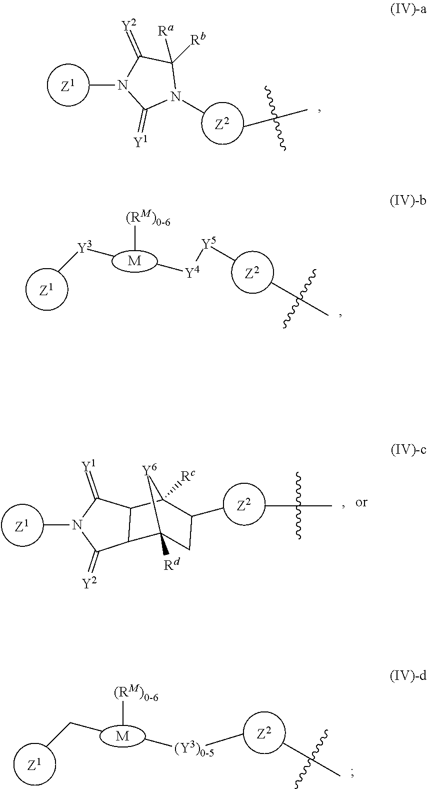

wherein:
Z¹ is selected from the group consisting of an aryl group, a heteroaryl group, a bicyclic group, or a bi-heterocyclic group, each independently substituted by one or more substituents independently selected from the group consisting of a halo group, a hydroxyl group, a nitro group, —CN, —C≡CH, an unsubstituted C₁₋₆ alkyl group, a C₁₋₆ alkyl group substituted by a C₁₋₆ alkoxyl group, a C₁₋₆ alkyl group substituted by one or more halo groups, an unsubstituted C₁₋₆ alkoxyl group, a C₁₋₆ alkoxyl group substituted by one or more halo groups, an unsubstituted C₂₋₆ alkenyl, a C₂₋₆ alkenyl substituted by one or more halo groups, an unsubstituted C₂₋₆ alkynyl, a C₃₋₆ alkynyl substituted by one or more halo groups, and any combinations thereof;
Y¹ and Y² are each independently NR^{Y1}, O or S;
Y³, Y⁴ and Y⁵ are each independently selected from the group consisting of a bond, —O—, —NR^{Y2}—, —C(—R^{Y1}) (—R^{Y2})—, —C(═O)—, —C(═S)—, —S(═O)—, —SO₂—, a heteroarylene group, and an arylene group;
Y⁶ is —N(—R^{Y1})—, —O— or —S—;
M is a 3- to 6-membered ring having 0 to 4 heteroatoms, which is unsubstituted or substituted by 0 to 6 R^{M} groups;
each R^{M} group is independently selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, and a C₁₋₆ alkoxyl group; or two R^{M} groups are taken together with the atom they attach to and form a 3- to 8-membered ring system containing 0 to 2 heteroatoms;
R^{a}, R^{b}, R^{c}, R^{d}, R^{Y1} and R^{Y2} are each independently selected from the group consisting of —H, an unsubstituted C₁₋₆ alkyl group, a C₁₋₆ alkyl group substituted by a C₁₋₆ alkoxyl group, a C₁₋₆ alkyl group substituted by one or more halo groups, a halo group, a C₁₋₆ alkoxyl group, a cyclic group, and a heterocyclic group; or R^{a}, R^{b} are taken together with the atom they attach to and form a 3- to 8-membered ring system containing 0 to 2 heteroatoms;
Z² is selected from the group consisting of a bond, a C₁₋₆ alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each or which is unsubstituted or substituted by 1 to 10 R^{Z2} groups;

each R^{Z2} group is independently selected from the group consisting of —H, a halo group, an unsubstituted C₁₋₆ alkyl group, a C₁₋₆ alkyl group substituted by one or more —F, —OR^{Z2A}, a C₃₋₆ cycloalkyl group, a C₄₋₆ cycloheteroalkyl group, an unsubstituted C₁₋₆ alkyl group, a C₁₋₆ alkyl group substituted by a C₁₋₃ alkyl group, a C₁₋₆ alkyl group substituted by a C₁₋₆ alkoxyl group, a C₁₋₆ alkyl group substituted by one or more halo groups, an unsubstituted heterocyclic group, a heterocyclic group substituted by a C₁₋₃ alkyl group, a heterocyclic group substituted by a C₁₋₆ alkoxyl group, a heterocyclic group substituted by one or more halo groups, an unsubstituted aryl group, an aryl group substituted by a C₁₋₃ alkyl group, an aryl group substituted by a C₁₋₆ alkoxyl group, an aryl group substituted by one or more halo groups, an unsubstituted heteroaryl group, a heteroaryl group substituted by a C₁₋₃ alkyl group, a heteroaryl group substituted by a C₁₋₆ alkoxyl group, a heteroaryl group substituted by one or more halo groups, a bicyclic heteroaryl group, an unsubstituted C₁₋₃ alkoxyl group, and a C₁₋₃ alkoxyl substituted by one or more groups selected from —F, —OH, —NH₂, —NR^{Y1}R^{Y2} and —CN; and
R^{Z2A} is selected from the group consisting of H, a C₁₋₆ alkyl group, and a C₁₋₆ heteroalkyl group, each of which is unsubstituted or substituted by a cycloalkyl group, a cycloheteroalkyl group, an aryl group, a heterocyclic group, a heteroaryl group, a halo group, or a C₁₋₃ alkoxyl group.

In some embodiments, the heteroatoms in Formula (IV)-a, (IV)-b, (IV)-c or (IV)-d are each independently selected from N, O and S.

In some embodiments, Z¹ is selected from the group consisting of

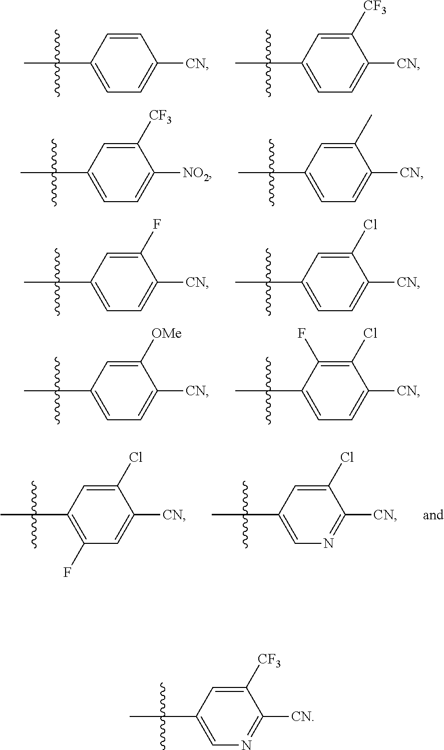

In some embodiments, Z¹ is selected from the group consisting of:

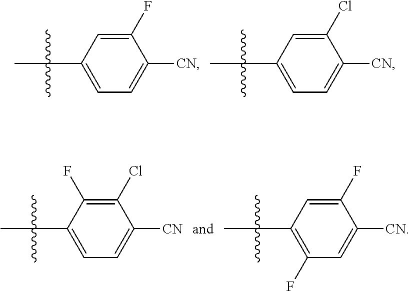 [SMILES: *c1ccc(C#N)c(F)c1 ; *c1ccc(C#N)c(Cl)c1 ; *c1ccc(C#N)c(Cl)c1F ; *c1cc(F)c(C#N)cc1F]

In some embodiments, Z² is selected from the group consisting of

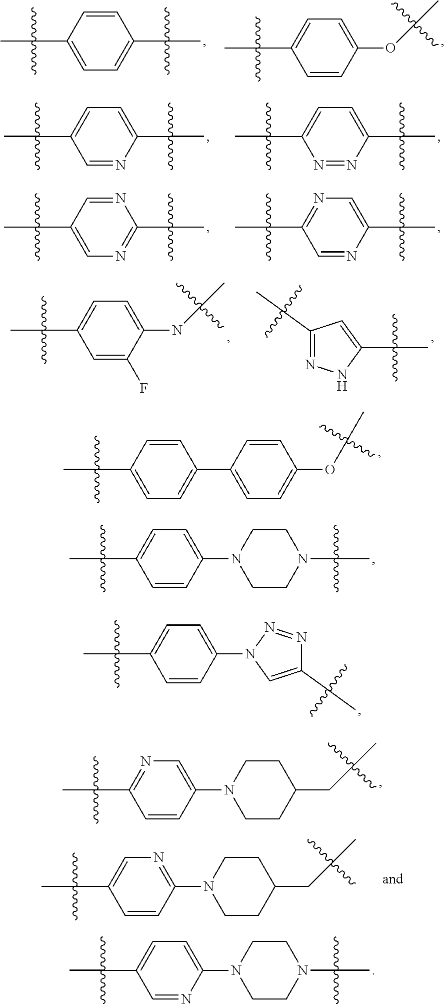

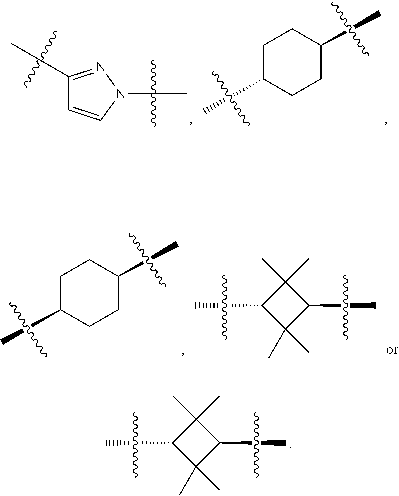

In some embodiments, M is

In some embodiments, each R^{M} group is independently an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, each R^{M} group is independently an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups. In some embodiments, each R^{M} group is independently methyl, ethyl, n-propyl or isopropyl.

In some embodiments, R^{a}, R^{b}, R^{c}, R^{d}, R^{Y1} and R^{Y2} are each independently an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, R^{a}, R^{b}, R^{c}, R^{d}, R^{Y1} and R^{Y2} are each independently an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups. In some embodiments, R^{a}, R^{b}, R^{c}, R^{d}, R^{Y1} and R^{Y2} are each independently methyl, ethyl n-propyl or isopropyl.

In some embodiments, (Y³)₀₋₅ shown in Formula (IV)-d is

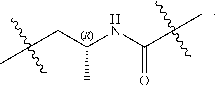

The mark (R) indicates that the carbon has R (rectus) configuration.

In some embodiments, each R^{Z2} group is independently an alkyl selected from the group consisting of a linear C₁₋₆ alkyl and a branched C₁₋₆ alkyl, each of which is unsubstituted or substituted by one or more halo groups. In some embodiments, each R^{Z2} group is independently an alkyl selected from the group consisting of a linear C₁₋₃ alkyl and a branched C₁₋₃ alkyl, each of which is unsubstituted or substituted by one or more halo groups.

In some embodiments, R^{Z2A} is an alkyl selected from the group consisting of a linear C₁₋₆ alkyl and a branched C₁₋₆ alkyl. In some embodiments, R^{Z2A} is an alkyl selected from the group consisting of a linear C₁₋₃ alkyl and a branched C₁₋₃ alkyl.

In some embodiments, R^{Z2A} is a heteroalkyl selected from the group consisting of a linear C₁₋₆ heteroalkyl and a branched C₁₋₆ heteroalkyl. In some embodiments, R^{Z2A} is a heteroalkyl selected from the group consisting of a linear C₁₋₃ heteroalkyl and a branched C₁₋₃ heteroalkyl.

In some embodiments, the ABM is selected from the group consisting of

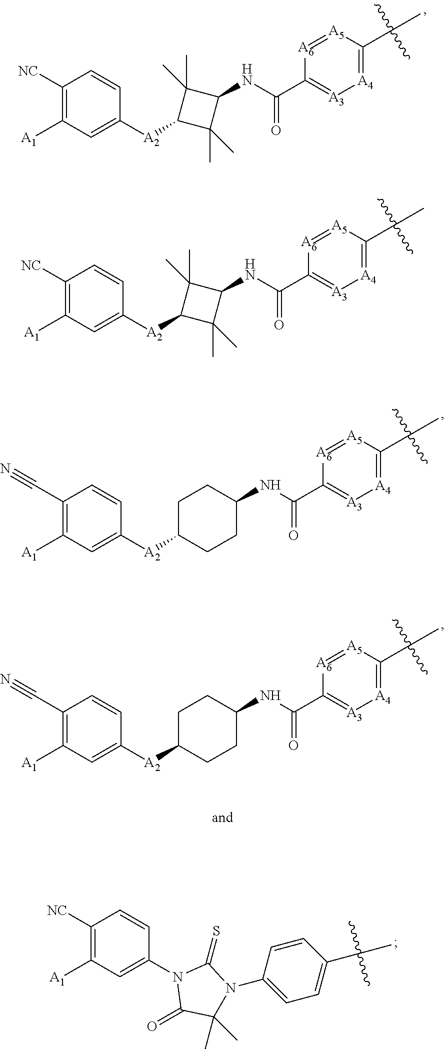

wherein:
A₁ is selected from —Cl, —F, —Br or —CF₃;
A₂ is selected from —O—, —NH—, —NCH₃— or —NCH₂CH₃—; and
A₃, A₄, A₅ and A₆ are each independently CH or N.

In certain embodiments, the ABM is an androgen receptor binding moiety represented by Formula (IV)-b, wherein M is a 4-membered alicyclic ring having 0 to 2 heteroatoms, which is unsubstituted or optionally substituted by 1 to 6 R^{M}. Other groups have the same meaning as indicated above.

In certain embodiments, the ABM is an androgen receptor binding moiety represented by Formula (IV)-b, wherein Z² is a bond, a C₁₋₆ alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each of which is substituted by 1, 2 or 3 R^{Z2} groups. Other groups have the same meaning as indicated above.

In certain embodiments, the ABM is an androgen receptor binding moiety represented by Formula (IV)-b, wherein
Z¹ is

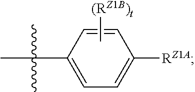

R^{ZA1} is —H or —CN;
each R^{Z1B} is independently —H, a halo group, or —CF₃; t is 0, 1, 2, 3 or 4;
Y³ is —O—;
M is a 4- to 6-membered ring, which is unsubstituted or substituted with 1 to 4 R^{M} groups, and each R^{M} is independently —H or methyl;
Y⁴ is —NH—;
Y⁵ is —C(═O)—;
Z² is independently selected from the group consisting of an unsubstituted C₅₋₆ aryl group, a C₅₋₆ aryl group substituted by 1, 2 or 3 R^{Z2} groups, a C₅₋₆ heteroaryl group having 1 to 2 heteroatoms, and a C₅₋₆ heteroaryl group having 1 heteroatom and substituted by 1, 2 or 3 R^{Z2} groups, and a C₅₋₆ heteroaryl group having 2 heteroatoms and substituted by 1 or 2 R^{Z2} groups; each R^{Z2A} group is an alkyl selected from the group consisting of a linear C₁₋₆ alkyl and a branched C₁₋₆ alkyl. In some embodiments, R^{Z2A} is an alkyl selected from the group consisting of a linear C₁₋₃ alkyl and a branched C₁₋₃ alkyl. Other groups have the same meaning as indicated above.

In certain embodiments, the ABM is an androgen receptor binding moiety represented by Formula (IV)-d, wherein
Z¹ is an aryl substituted by one or more halo groups, or an aryl substituted by —CN; or an aryl independently substituted by —CN and one or more halo groups;
Y³ is a bond, —NR^{Y2}—, —CR^{Y1}R^{Y2}—, or —C(═O)—;
M is a 5-membered aromatic ring having 1 or 2 heteroatoms;
R^{Y1} and R^{Y2} are each independently H, or a C₁₋₆ alkyl group;
Z² is a bond, an aryl, or heteroaryl, each optionally substituted by 1, 2 or 3 R^{W2}; and each R^{W2} is independently —H, a halo group, C₁₋₆ alkyl (optionally substituted by 1 or more —F), C₁₋₃ alkoxyl (optionally substituted by 1 or more —F):
R^{W2} group is independently selected from the group consisting of —H, a halo group, a 6-membered alicyclic group having 1 or 2 heteroatoms, or a 5-membered aromatic group having 1 or 2 or 3 heteroatoms. Other groups have the same meaning as indicated above.

In certain embodiments, the ABM is an androgen receptor binding moiety represented by Formula (IV)-d, wherein
Z¹ is

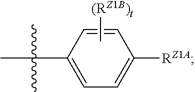

wherein each R^{Z1A} is a halo group or CN; and each R^{Z1B} is independently H or halo. Other groups have the same meaning as indicated above.

In some embodiments, ABM is selected from the group consisting of

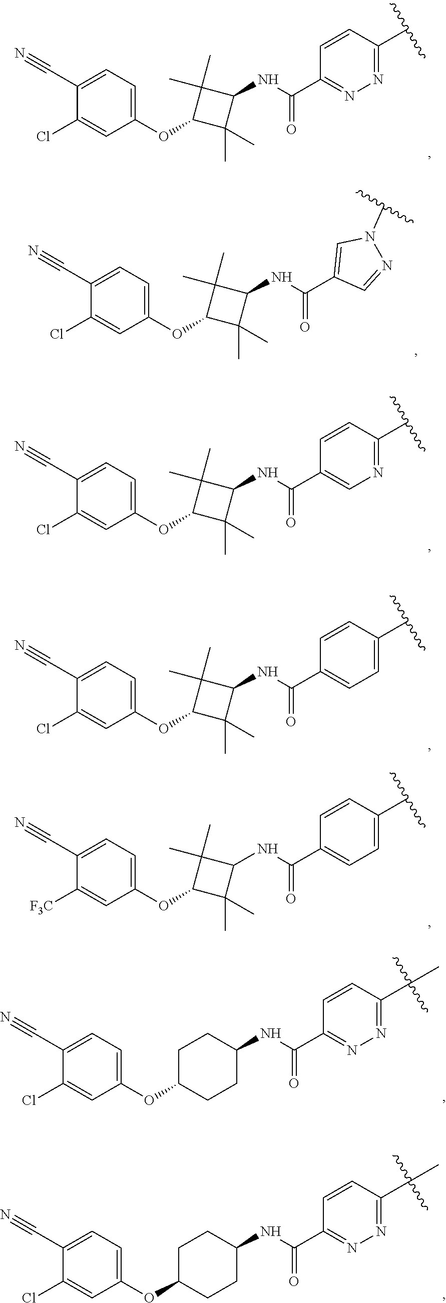

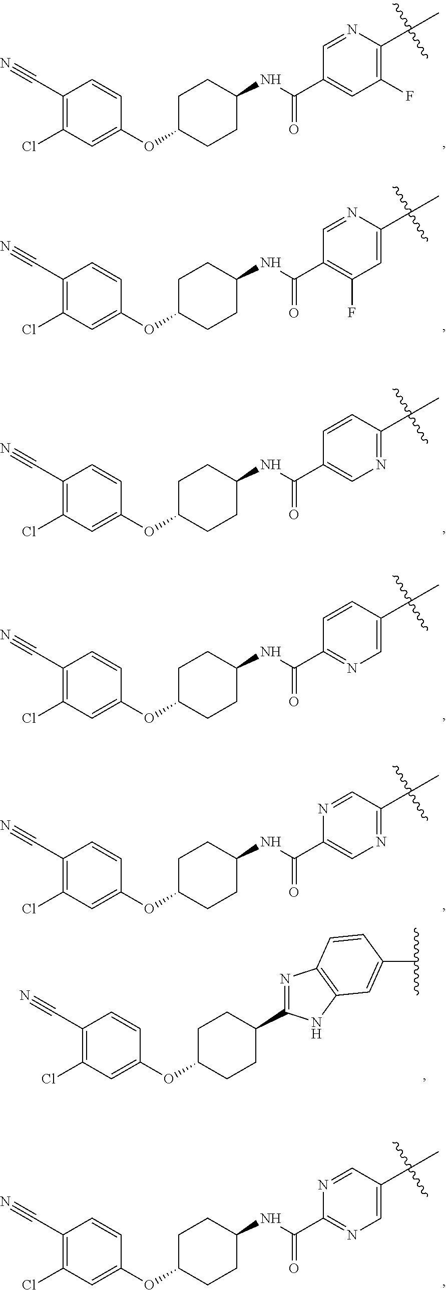

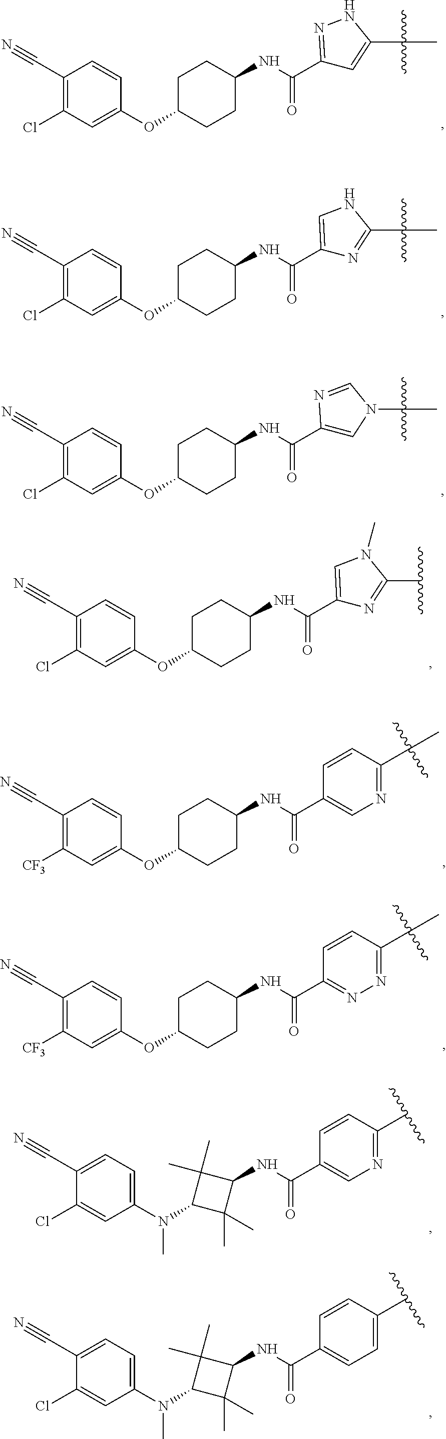

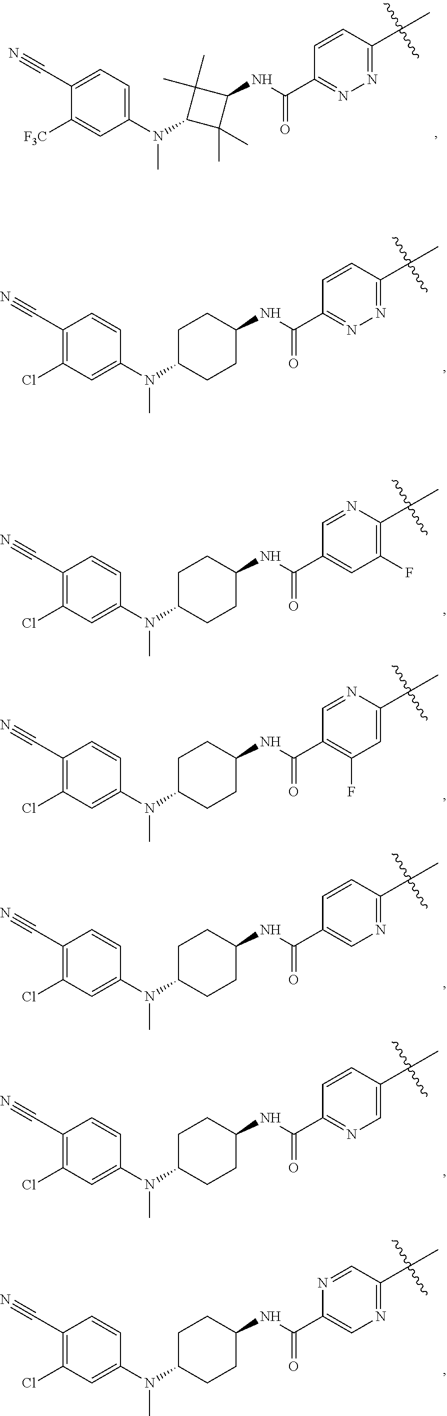

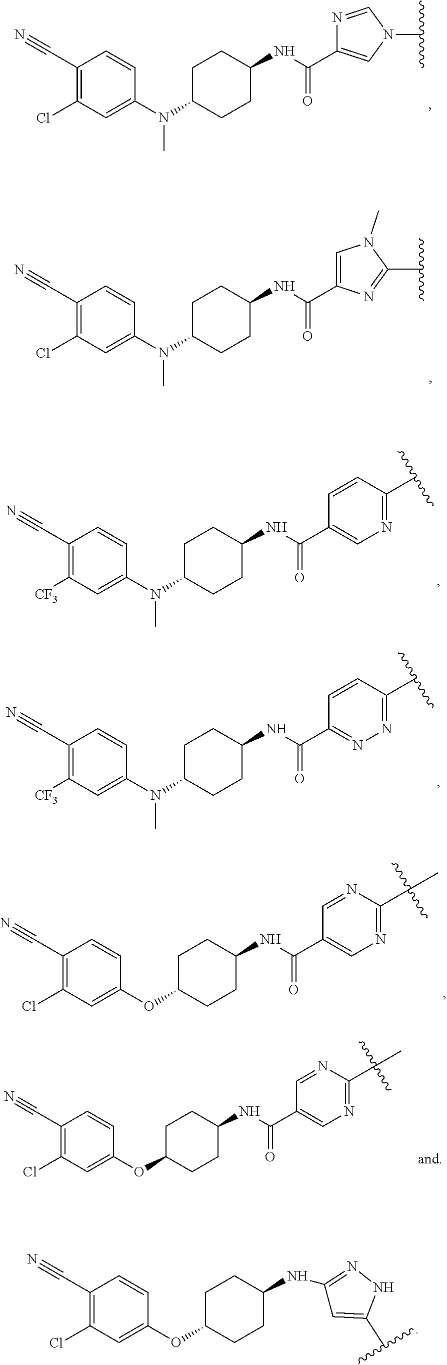

In some embodiments, the pharmaceutical composition of the present invention further comprises a second therapeutic agent.

In some embodiments, the androgen receptor related disease is an androgen receptor related cancer or an androgen receptor related skin disease.

In some embodiments, the androgen receptor related cancer is breast cancer or prostate cancer. In some embodiments, the cancer is breast cancer. In some embodiments, the cancer is prostate cancer. In some embodiments, the prostate cancer is castration-resistant prostate cancer.

In some embodiments, the androgen receptor related skin disease is androgenetic alopecia, acne, hidradenitis suppurativa, hirsutism, or atopic dermatitis.

In some embodiments, the method for treating an androgen receptor related disease further comprises administering an effective amount of a second therapeutic agent.

In some embodiments, the second therapeutic agent may be an androgen receptor inhibitor. Examples of the androgen receptor inhibitor may be enzalutamide.

In some embodiments, the second therapeutic agent may be an antitumor agent conventionally used in the art. Examples of conventional antitumor agents include Docetaxel, Flutamide, Goserelin acetate, Leuprolide acetate, colchicine, Leuprolide acetate, Mitoxantrone hydrochloride, 5-fluorouracil, and Olaparib. Examples thereof include one or more of cyclophosphamide, mitomycin C and the like.

In some embodiments, the second therapeutic agent may be a therapeutic agent for treating an AR related skin disease conventionally used in the art. Examples of conventional antitumor agents include Clascoterone, ASC-J9, Spironolactone, Flutamide, Finasteride, dutasteride, Cyproterone acetate, Pyrilutamide, Minoxidil, Ketoconazole and the like.

As used herein in the specification and in the claims, the articles “a” and “an” are used to refer to one or to more than one (i.e., to at least one) of the grammatical object of the article unless the context clearly indicates otherwise. By way of example, “an element” means one element or more than one element.

As used herein in the specification and in the claims, the phrase “and/or,” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a condition, disorder or disease state in a subject in need of such treatment or receiving such treatment. The term “effective” subsumes all other effective amount or effective concentration terms, e.g., “effective amount/dose,” “pharmaceutically effective amount/dose” or “therapeutically effective amount/dose,” which are otherwise described or used in the present application.

The effective amount depends on the type and severity of disease, the composition used, the route of administration, the type of mammal being treated, the physical characteristics of the specific mammal under consideration, concurrent medication, and other factors which those skilled in the medical arts will recognize. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described.

It is understood by those of ordinary skill in the art that molecules which are described herein are stable compounds as generally described hereunder. When the bond [symbol] is shown, both a double bond and single bond are represented or understood within the context of the compound shown and well-known rules for valence interactions.

As used herein, an “alkoxyl group” refers to an alkyl group which is singularly bonded to oxygen; such as methoxy (—O—CH₃) and ethoxy (—O—CH₂CH₃).

As used herein, “derivatives” can mean compositions formed from the native compounds either directly, by modification, or by partial substitution. As used herein, “analogs” can mean compositions that have a structure similar to, but not identical to, the native compound.

The term “ubiquitin ligase” refers to a family of proteins that facilitate the transfer of ubiquitin to a specific substrate protein, targeting the substrate protein for degradation. For example, cereblonE3 ubiquitin ligase, alone or in combination with E2 ubiquitin-conjugating enzyme, causes the attachment of ubiquitin to lysine on a target protein, and subsequently targets the specific protein substrates for degradation by the proteasome. Thus, E3 ubiquitin ligase, alone or in complex with E2 ubiquitin conjugating enzyme, is responsible for the transfer of ubiquitin to target proteins. In general, the ubiquitin ligases are involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further, E3 ubiquitin ligase promotes the formation of poly-ubiquitin chains through the lysine residues on ubiquitin. Lys48-linked chains are the predominant type of poly-ubiquitination, which can target proteins to proteasome for degradation.

The term “patient” or “subject” is used throughout the specification to describe a cell, tissue, or animal, preferably a mammal, e.g., a human or a domesticated animal, to whom treatment, including prophylactic treatment, with the compositions according to the present disclosure is provided. For treatment of those infections, conditions or disease states which are specific for a specific animal such as a human patient, the term “patient” refers to that specific animal, including a domesticated animal such as a dog or cat or a farm animal such as a horse, cow, sheep, etc. In general, in the present disclosure, the term “patient” refers to a human patient unless otherwise stated or implied from the context of the use of the term.

Understand that in all cases disclosed herein, the description of an integer range of any variable describes the description range, all individual members of the range, and all possible subranges of that variable. For example, the description that n is an integer from 0 to 4 is 0, 1, 2, 3 and 4 are described as being in the range of individual selectable values. In addition, the description that n is an integer from 0 to 4 also describes each and all subranges, each of which n is 0-4, 0-3, 0-2, 0-1, 1-4, 1-3, 1-2, 2-4, 2-3 and 3-4. In addition, the term “C₁₋₆ alkyl” indicates an alkyl having a carbon number of 1 to 6.

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

### DETAILED DESCRIPTION OF THE PREFERRED EMBODIMENTS

Shown in Table 1 below are exemplary of the bifunctional compounds with the structure ABM -L- CLM of the present invention:

TABLE 1
Structure

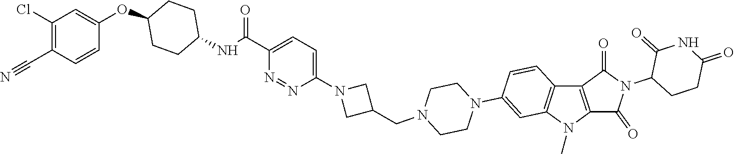 [SMILES: Cn1c2c(c3ccc(N4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 1

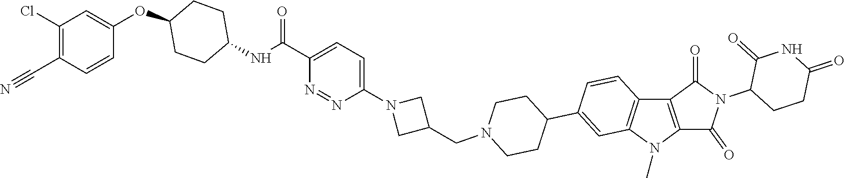 [SMILES: Cn1c2c(c3ccc(C4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 2

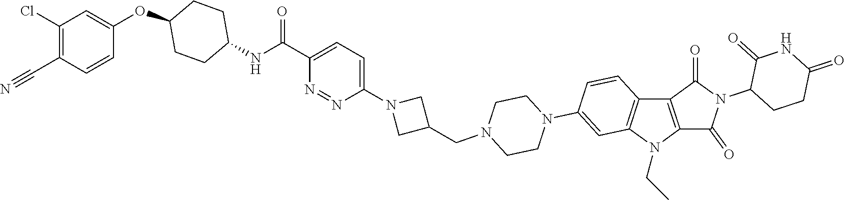 [SMILES: CCn1c2c(c3ccc(N4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 3

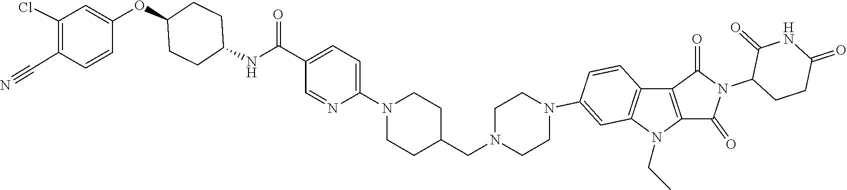 [SMILES: CCn1c2c(c3ccc(N4CCN(CC5CCN(c6ccc(C(=O)N[C@H]7CC[C@H](Oc8ccc(C#N)c(Cl)c8)CC7)cn6)CC5)CC4)cc31)C(=O)N(C1CCC(=O)NC1=O)C2=O]

Compound 4

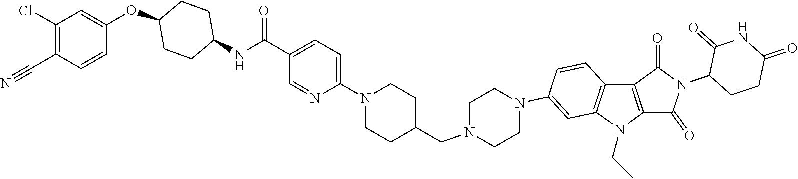 [SMILES: CCn1c2c(c3ccc(N4CCN(CC5CCN(c6ccc(C(=O)N[C@H]7CC[C@@H](Oc8ccc(C#N)c(Cl)c8)CC7)cn6)CC5)CC4)cc31)C(=O)N(C1CCC(=O)NC1=O)C2=O]

Compound 5

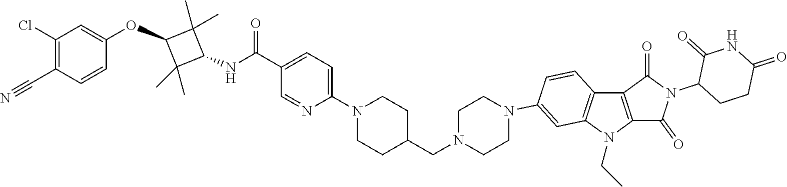 [SMILES: CCn1c2c(c3ccc(N4CCN(CC5CCN(c6ccc(C(=O)N[C@H]7C(C)(C)[C@H](Oc8ccc(C#N)c(Cl)c8)C7(C)C)cn6)CC5)CC4)cc31)C(=O)N(C1CCC(=O)NC1=O)C2=O]

Compound 6

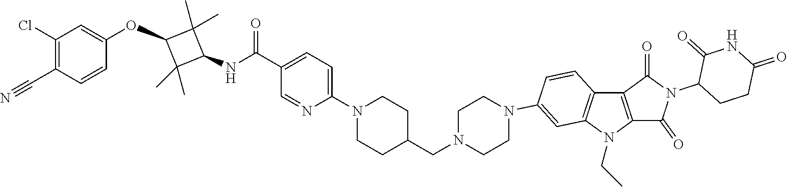 [SMILES: CCn1c2c(c3ccc(N4CCN(CC5CCN(c6ccc(C(=O)N[C@H]7C(C)(C)[C@@H](Oc8ccc(C#N)c(Cl)c8)C7(C)C)cn6)CC5)CC4)cc31)C(=O)N(C1CCC(=O)NC1=O)C2=O]

Compound 7

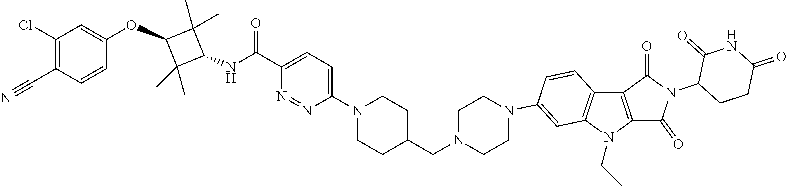 [SMILES: CCn1c2c(c3ccc(N4CCN(CC5CCN(c6ccc(C(=O)N[C@H]7C(C)(C)[C@H](Oc8ccc(C#N)c(Cl)c8)C7(C)C)nn6)CC5)CC4)cc31)C(=O)N(C1CCC(=O)NC1=O)C2=O]

Compound 8

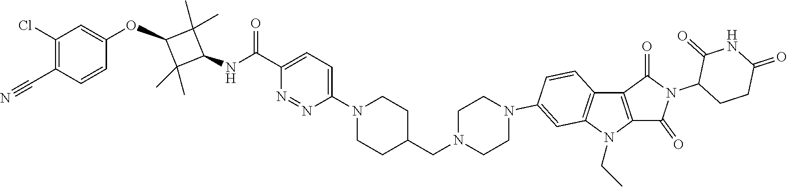 [SMILES: CCn1c2c(c3ccc(N4CCN(CC5CCN(c6ccc(C(=O)N[C@H]7C(C)(C)[C@@H](Oc8ccc(C#N)c(Cl)c8)C7(C)C)nn6)CC5)CC4)cc31)C(=O)N(C1CCC(=O)NC1=O)C2=O]

Compound 9

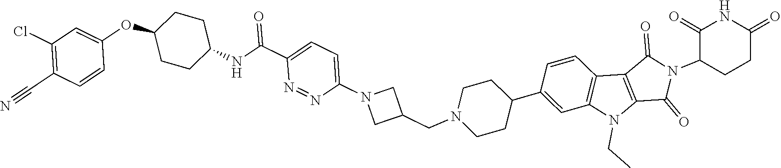 [SMILES: CCn1c2c(c3ccc(C4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 10

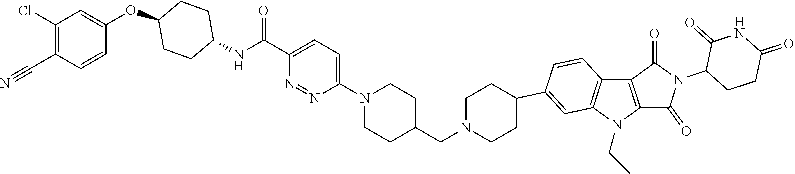 [SMILES: CCn1c2c(c3ccc(C4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 11

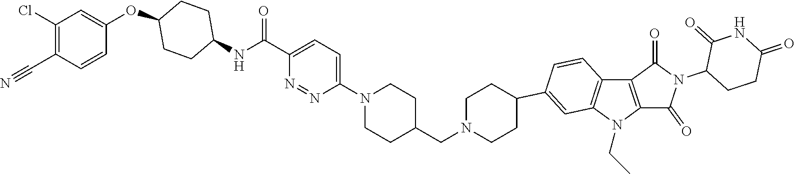 [SMILES: CCn1c2c(c3ccc(C4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O]

Compound 12

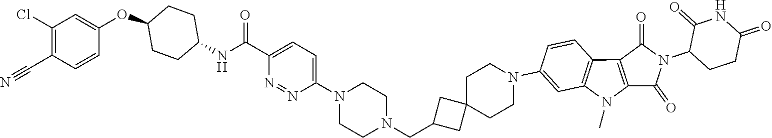 [SMILES: Cn1c2c(c3ccc(N4CCC5(CC4)CC(CN4CCN(c6ccc(C(=O)N[C@H]7CC[C@H](Oc8ccc(C#N)c(Cl)c8)CC7)nn6)CC4)C5)cc31)C(=O)N(C1CCC(=O)NC1=O)C2=O]

Compound 13

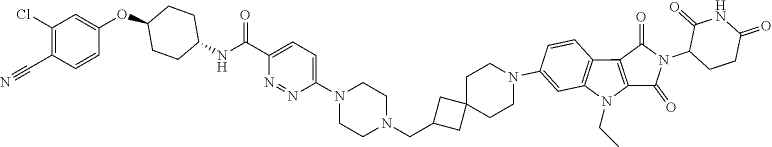 [SMILES: CCn1c2c(c3ccc(N4CCC5(CC4)CC(CN4CCN(c6ccc(C(=O)N[C@H]7CC[C@H](Oc8ccc(C#N)c(Cl)c8)CC7)nn6)CC4)C5)cc31)C(=O)N(C1CCC(=O)NC1=O)C2=O]

Compound 14

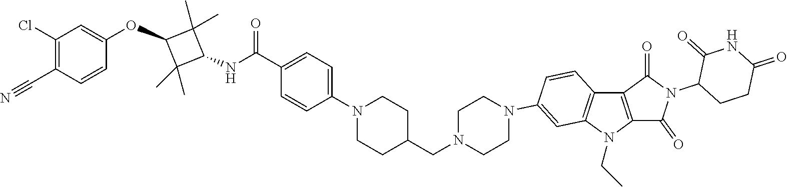 [SMILES: CCn1c2c(c3ccc(N4CCN(CC5CCN(c6ccc(C(=O)N[C@H]7C(C)(C)[C@H](Oc8ccc(C#N)c(Cl)c8)C7(C)C)cc6)CC5)CC4)cc31)C(=O)N(C1CCC(=O)NC1=O)C2=O]

Compound 15

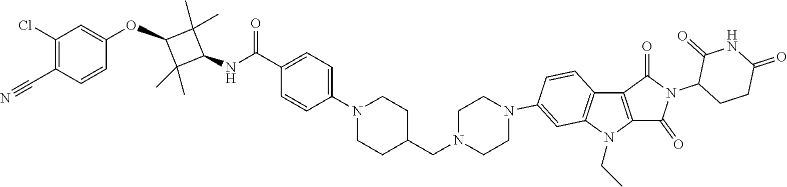 [SMILES: CCn1c2c(c3ccc(N4CCN(CC5CCN(c6ccc(C(=O)N[C@H]7C(C)(C)[C@@H](Oc8ccc(C#N)c(Cl)c8)C7(C)C)cc6)CC5)CC4)cc31)C(=O)N(C1CCC(=O)NC1=O)C2=O]

Compound 16

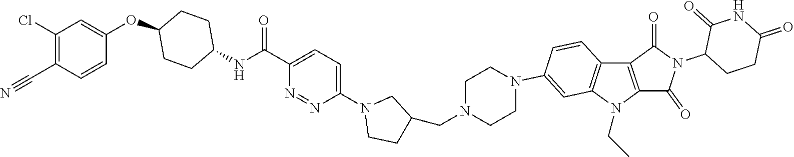 [SMILES: CCn1c2c(c3ccc(N4CCN(CC5CCN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 17

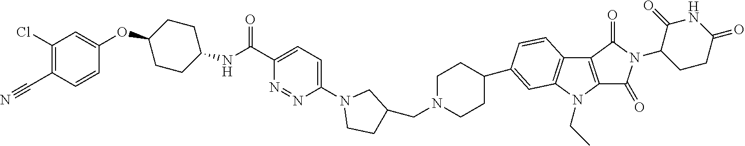 [SMILES: CCn1c2c(c3ccc(C4CCN(CC5CCN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 18

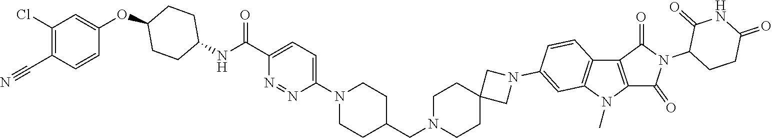 [SMILES: Cn1c2c(c3ccc(N4CC5(CCN(CC6CCN(c7ccc(C(=O)N[C@H]8CC[C@H](Oc9ccc(C#N)c(Cl)c9)CC8)nn7)CC6)CC5)C4)cc31)C(=O)N(C1CCC(=O)NC1=O)C2=O]

Compound 19

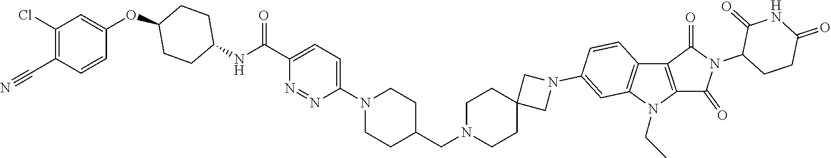 [SMILES: CCn1c2c(c3ccc(N4CC5(CCN(CC6CCN(c7ccc(C(=O)N[C@H]8CC[C@H](Oc9ccc(C#N)c(Cl)c9)CC8)nn7)CC6)CC5)C4)cc31)C(=O)N(C1CCC(=O)NC1=O)C2=O]

Compound 20

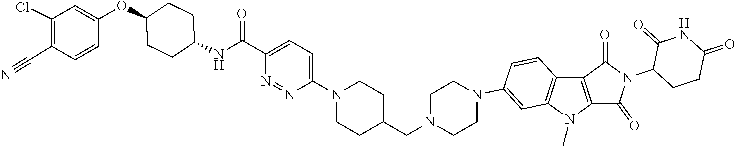 [SMILES: Cn1c2c(c3ccc(N4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 21

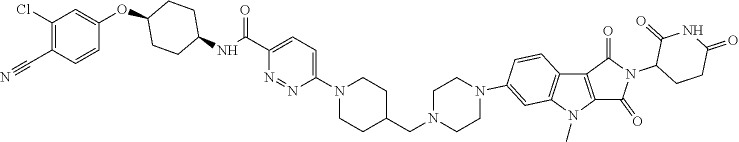 [SMILES: Cn1c2c(c3ccc(N4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O]

Compound 22

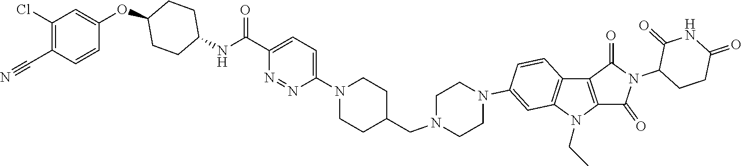 [SMILES: CCn1c2c(c3ccc(N4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 23

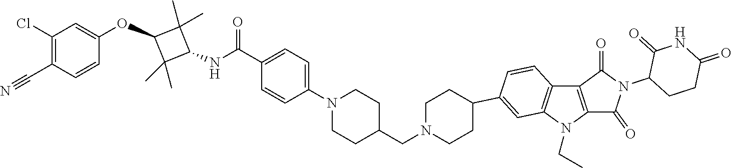 [SMILES: CCn1c2c(c3ccc(C4CCN(CC5CCN(c6ccc(C(=O)N[C@H]7C(C)(C)[C@H](Oc8ccc(C#N)c(Cl)c8)C7(C)C)cc6)CC5)CC4)cc31)C(=O)N(C1CCC(=O)NC1=O)C2=O]

Compound 24

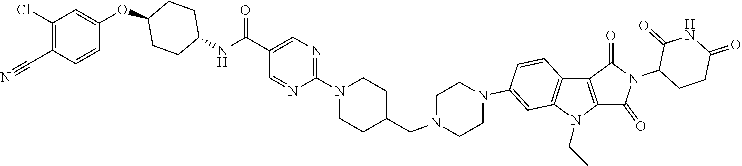 [SMILES: CCn1c2c(c3ccc(N4CCN(CC5CCN(c6ncc(C(=O)N[C@H]7CC[C@H](Oc8ccc(C#N)c(Cl)c8)CC7)cn6)CC5)CC4)cc31)C(=O)N(C1CCC(=O)NC1=O)C2=O]

Compound 25

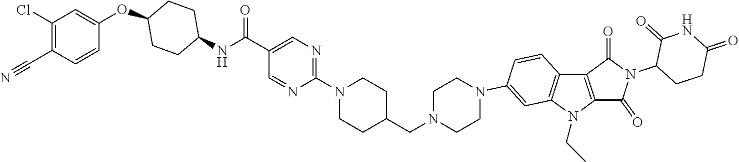 [SMILES: CCn1c2c(c3ccc(N4CCN(CC5CCN(c6ncc(C(=O)N[C@H]7CC[C@@H](Oc8ccc(C#N)c(Cl)c8)CC7)cn6)CC5)CC4)cc31)C(=O)N(C1CCC(=O)NC1=O)C2=O]

Compound 26

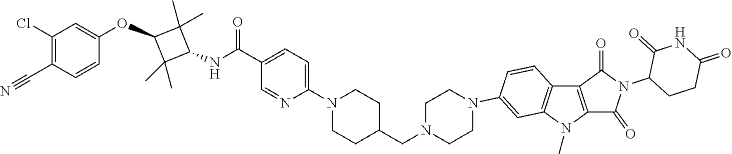 [SMILES: Cn1c2c(c3ccc(N4CCN(CC5CCN(c6ccc(C(=O)N[C@H]7C(C)(C)[C@H](Oc8ccc(C#N)c(Cl)c8)C7(C)C)cn6)CC5)CC4)cc31)C(=O)N(C1CCC(=O)NC1=O)C2=O]

Compound 27

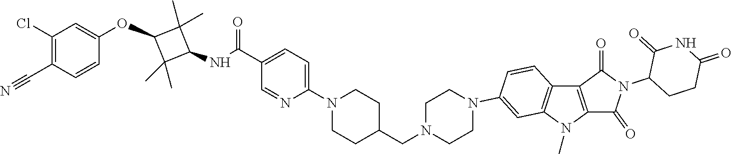 [SMILES: Cn1c2c(c3ccc(N4CCN(CC5CCN(c6ccc(C(=O)N[C@H]7C(C)(C)[C@@H](Oc8ccc(C#N)c(Cl)c8)C7(C)C)cn6)CC5)CC4)cc31)C(=O)N(C1CCC(=O)NC1=O)C2=O]

Compound 28

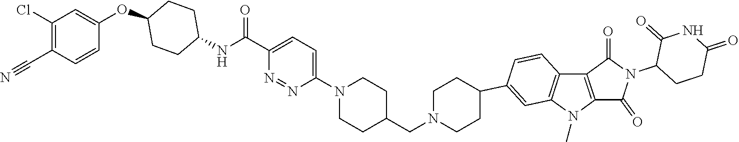 [SMILES: Cn1c2c(c3ccc(C4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 29

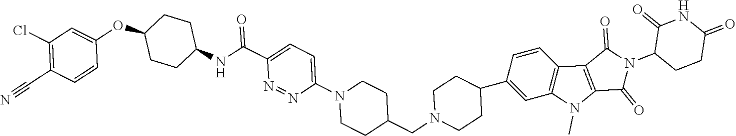 [SMILES: Cn1c2c(c3ccc(C4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O]

Compound 30

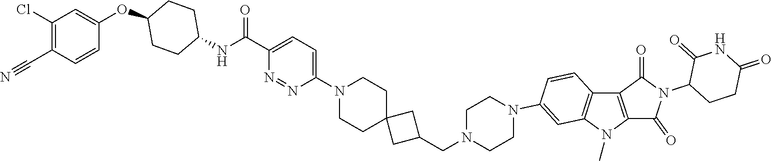 [SMILES: Cn1c2c(c3ccc(N4CCN(CC5CC6(CCN(c7ccc(C(=O)N[C@H]8CC[C@H](Oc9ccc(C#N)c(Cl)c9)CC8)nn7)CC6)C5)CC4)cc31)C(=O)N(C1CCC(=O)NC1=O)C2=O]

Compound 31

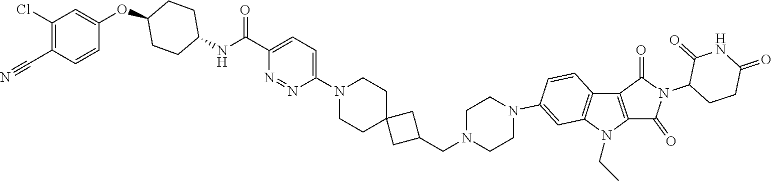 [SMILES: CCn1c2c(c3ccc(N4CCN(CC5CC6(CCN(c7ccc(C(=O)N[C@H]8CC[C@H](Oc9ccc(C#N)c(Cl)c9)CC8)nn7)CC6)C5)CC4)cc31)C(=O)N(C1CCC(=O)NC1=O)C2=O]

Compound 32

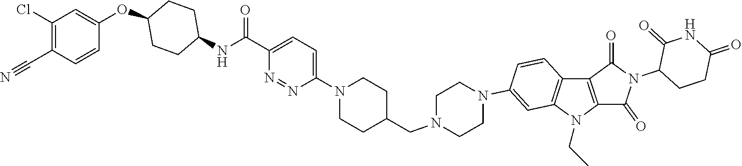 [SMILES: CCn1c2c(c3ccc(N4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O]

Compound 33

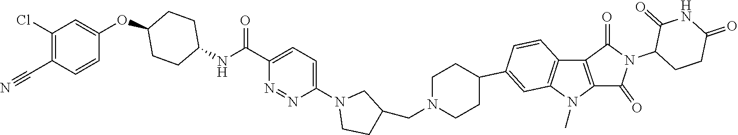 [SMILES: Cn1c2c(c3ccc(C4CCN(CC5CCN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 34

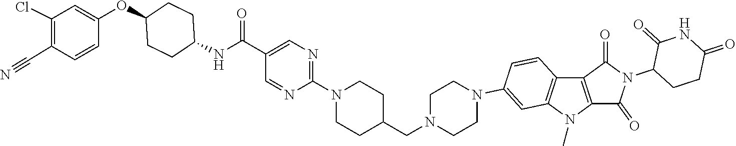 [SMILES: Cn1c2c(c3ccc(N4CCN(CC5CCN(c6ncc(C(=O)N[C@H]7CC[C@H](Oc8ccc(C#N)c(Cl)c8)CC7)cn6)CC5)CC4)cc31)C(=O)N(C1CCC(=O)NC1=O)C2=O]

Compound 35

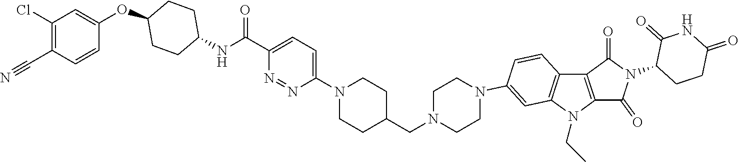 [SMILES: CCn1c2c(c3ccc(N4CCN(CC5CCN(c6ccc(C(=O)N[C@H]7CC[C@H](Oc8ccc(C#N)c(Cl)c8)CC7)nn6)CC5)CC4)cc31)C(=O)N([C@H]1CCC(=O)NC1=O)C2=O]

Compound 36

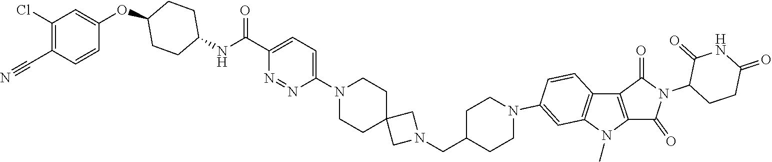 [SMILES: Cn1c2c(c3ccc(N4CCC(CN5CC6(CCN(c7ccc(C(=O)N[C@H]8CC[C@H](Oc9ccc(C#N)c(Cl)c9)CC8)nn7)CC6)C5)CC4)cc31)C(=O)N(C1CCC(=O)NC1=O)C2=O]

Compound 37

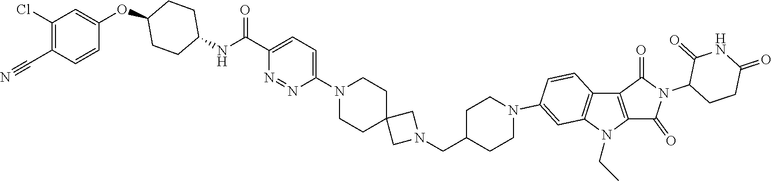 [SMILES: CCn1c2c(c3ccc(N4CCC(CN5CC6(CCN(c7ccc(C(=O)N[C@H]8CC[C@H](Oc9ccc(C#N)c(Cl)c9)CC8)nn7)CC6)C5)CC4)cc31)C(=O)N(C1CCC(=O)NC1=O)C2=O]

Compound 38

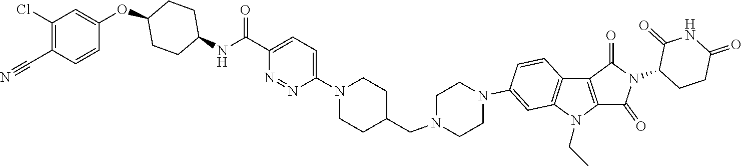 [SMILES: CCn1c2c(c3ccc(N4CCN(CC5CCN(c6ccc(C(=O)N[C@H]7CC[C@@H](Oc8ccc(C#N)c(Cl)c8)CC7)nn6)CC5)CC4)cc31)C(=O)N([C@H]1CCC(=O)NC1=O)C2=O]

Compound 39

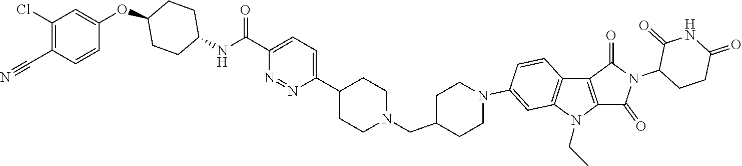 [SMILES: CCn1c2c(c3ccc(N4CCC(CN5CCC(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 40

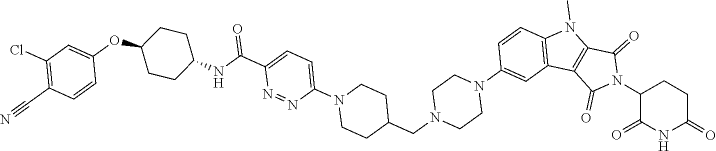 [SMILES: Cn1c2c(c3cc(N4CCN(CC5CCN(c6ccc(C(=O)N[C@H]7CC[C@H](Oc8ccc(C#N)c(Cl)c8)CC7)nn6)CC5)CC4)ccc31)C(=O)N(C1CCC(=O)NC1=O)C2=O]

Compound 41

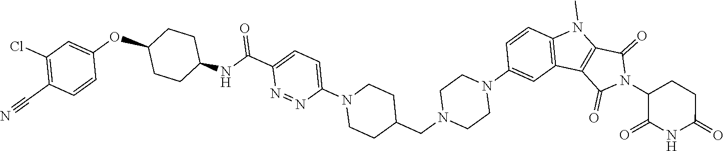 [SMILES: Cn1c2c(c3cc(N4CCN(CC5CCN(c6ccc(C(=O)N[C@H]7CC[C@@H](Oc8ccc(C#N)c(Cl)c8)CC7)nn6)CC5)CC4)ccc31)C(=O)N(C1CCC(=O)NC1=O)C2=O]

Compound 42

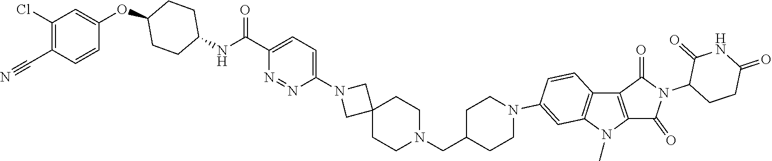 [SMILES: Cn1c2c(c3ccc(N4CCC(CN5CCC6(CC5)CN(c5ccc(C(=O)N[C@H]7CC[C@H](Oc8ccc(C#N)c(Cl)c8)CC7)nn5)C6)CC4)cc31)C(=O)N(C1CCC(=O)NC1=O)C2=O]

Compound 43

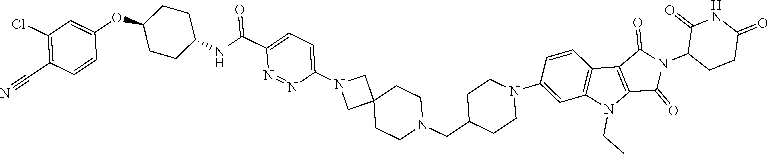 [SMILES: CCn1c2c(c3ccc(N4CCC(CN5CCC6(CC5)CN(c5ccc(C(=O)N[C@H]7CC[C@H](Oc8ccc(C#N)c(Cl)c8)CC7)nn5)C6)CC4)cc31)C(=O)N(C1CCC(=O)NC1=O)C2=O]

Compound 44

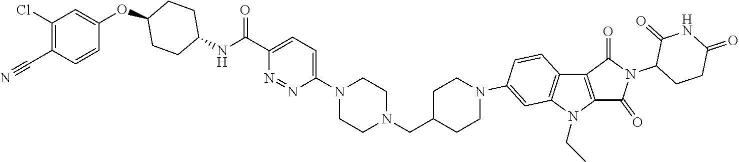 [SMILES: CCn1c2c(c3ccc(N4CCC(CN5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 45

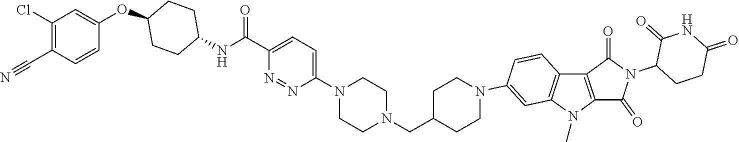 [SMILES: Cn1c2c(c3ccc(N4CCC(CN5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 46

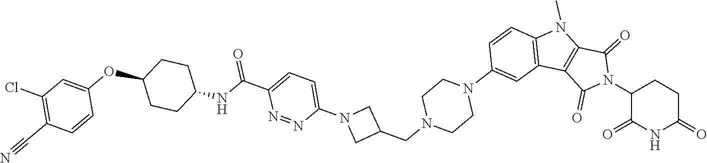 [SMILES: Cn1c2c(c3cc(N4CCN(CC5CN(c6ccc(C(=O)N[C@H]7CC[C@H](Oc8ccc(C#N)c(Cl)c8)CC7)nn6)C5)CC4)ccc31)C(=O)N(C1CCC(=O)NC1=O)C2=O]

Compound 47

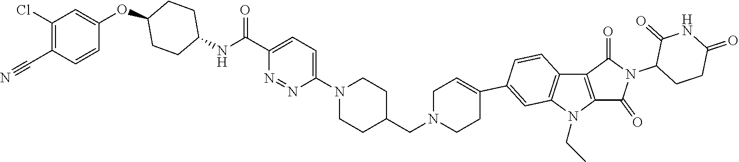 [SMILES: CCn1c2c(c3ccc(C4=CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O]

Compound 48

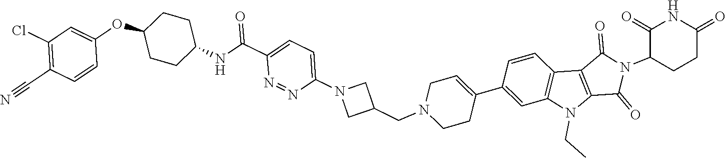 [SMILES: CCn1c2c(c3ccc(C4=CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

Compound 49

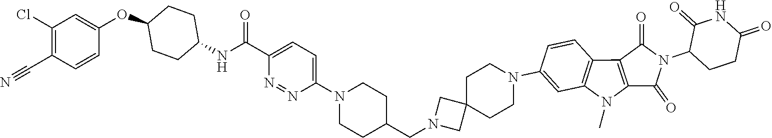 [SMILES: Cn1c2c(c3ccc(N4CCC5(CC4)CN(CC4CCN(c6ccc(C(=O)N[C@H]7CC[C@H](Oc8ccc(C#N)c(Cl)c8)CC7)nn6)CC4)C5)cc31)C(=O)N(C1CCC(=O)NC1=O)C2=O]

Compound 50

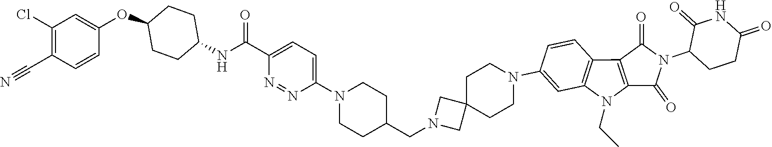 [SMILES: CCn1c2c(c3ccc(N4CCC5(CC4)CN(CC4CCN(c6ccc(C(=O)N[C@H]7CC[C@H](Oc8ccc(C#N)c(Cl)c8)CC7)nn6)CC4)C5)cc31)C(=O)N(C1CCC(=O)NC1=O)C2=O]

Compound 51

### Method for Producing Compound of the Present Invention

A general method for producing the compounds of the present invention will be exemplified below. And, as extraction and purification, treatment which is performed in a normal experiment of organic chemistry may be conducted.

Synthesis of the compound of the present invention can be carried out referring to the procedures known in the art.

As a raw material compound, commercially available compounds, compounds described in the present description, compounds described in the references cited in the present description, and other known compounds can be utilized.

Among the compounds of the present invention, there are compounds in which a tautomer can be present, and the present invention includes all possible isomers and a mixture thereof, including them.

When one wants to obtain a salt of the compound of the present invention, in the case where the compound of the present invention is obtained in a form of a salt, it may be purified as it is and, in the case where the compound of the present invention is obtained in a free form, a salt may be formed by a normal method by dissolving or suspending the compound in a suitable organic solvent, and adding an acid or a base.

In addition, the compound of the present invention and a pharmaceutically acceptable salt thereof are present in a form of adducts with water or various solvents (hydrate or solvate) in some cases, and these adducts are included in the present invention.

The details of one or more embodiments of the invention are set forth in the description below. Other features, objects, and advantages of the invention will be apparent from the description and from the claims.

The compounds of this invention can be synthesized from commercially available starting materials by methods well known in the art. For example, one can prepare the compounds of this invention via the route shown below:

### General Procedure for the Compound Preparation:

### Procedure 1

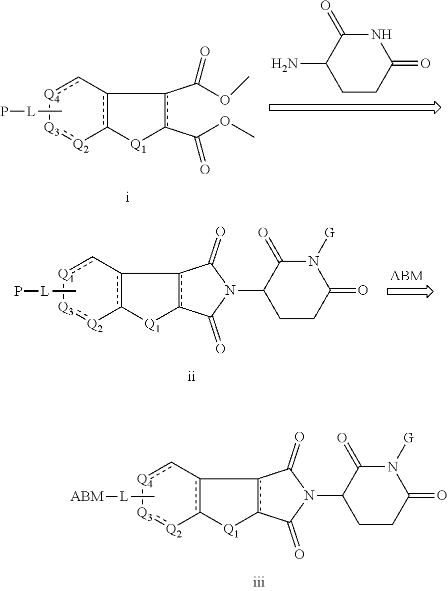

The Procedure 1 illustrates a general process for the preparation of the bifunctional compound of the present disclosure. In the first stage, a di-ester compound (i) was condensed with 3-aminopiperidine-2,6-dione to form an intermediate (ii). In the second stage, the intermediate (ii) was further condensed with a specific ABM compound to afford the desired compound (iii). The letter “P”, shown in the above Procedure 1, represents a protecting group.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Synthetic Method A—Synthesis of Compound 1:

### (1) Preparation of Intermediate I-A

The intermediate I-A was first prepared from commercially available 3-benzyloxy-phenylamine via the route shown below:

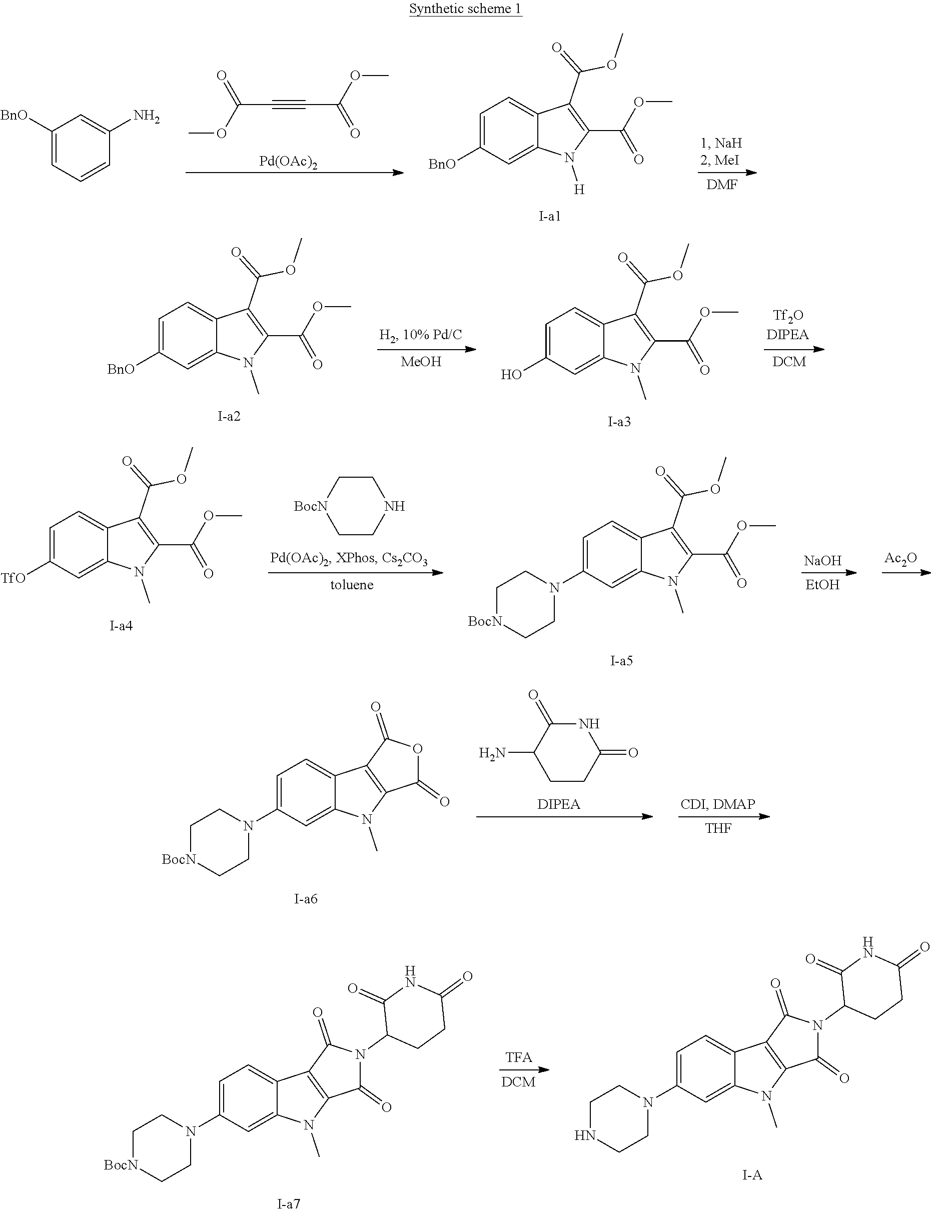

Pd(OAc)₂ (1.13 g, 10 mol %) was added to a solution of 3-benzyloxy-phenylamine (10 g, 50.1 mmol), dimethyl acetylenedicarboxylate (5.86 mL, 47.6 mmol), and dimethylacetamide (DMA)/pivalic acid (PivOH) (4:1 v/v; 100 mL) in a container purged with air. The reaction mixture was gradually heated to 120° C. for 8 h. After reaction was completed, the solution was then cooled to room temperature, diluted with ethyl acetate (200 mL), washed with water (3×50 mL) and brine (50 mL), dried over MgSO₄, filtered, and evaporated under vacuum to give a crude product. The crude product was purified by column chromatography on silica gel (KM3 SCIENTIFIC CORP., Particle size 45-75 μm) with an eluent (hexane:ethyl acetate=5:1) to afford a brown solid of 6-benzyloxy-1H-indole-2,3-dicarboxylic acid dimethyl ester I-a1 (8.4 g, 54%)

An ice cold solution of 6-benzyloxy-1H-indole-2,3-dicarboxylic acid dimethyl ester I-a1 (3.0 g, 8.84 mmol) in N,N-dimethylformamide (DMF) (15 ml) was treated with 60% NaH (0.53 g, 13.3 mmol) and methyl iodide (0.82 ml, 13.3 mmol) and then warmed to room temperature. After 1 h, the reaction mixture was partitioned between dichloromethane (DCM) (60 mL) and saturated aqueous NH₄Cl (20 mL). The combined organic layers were collected and washed with water (3×20 mL) and brine (20 mL), then dried over MgSO₄, filtered and concentrated to give a crude product. The crude product was purified by column chromatography on silica gel (KM3 SCIENTIFIC CORP., Particle size 45-75 μm) with an eluent (hexane:ethyl acetate=3:1) to afford a brown solid I-a2 (2.7 g, 87%).

To a solution of 6-benzyloxy-1-methyl-1H-indole-2,3-dicarboxylic acid dimethyl ester I-a2 (5.0 g) in MeOH (150 mL), Pd/C (0.5 g) was added and stirred under hydrogen atmosphere for 2 h. After the reaction was completed, the mixture was passed through a Celite pad and rinsed with ethyl acetate (10 mL), and the organic solvent was collected and removed on a rotor to afford an intermediate I-a3 (3.72 g, 100%).

To a stirred solution of 6-hydroxy-1-methyl-1H-indole-2,3-dicarboxylic acid dimethyl ester I-a3 (2.85 g, 10.83 mmol, 1.0 eq) in DCM (60 mL) was added N,N-diisopropylethylamine (DIPEA) (5.6 mL, 32.49 mmol, 3 eq) at 0° C. and the mixture was stirred at the same temperature for 10 minutes. Trifluoromethanesulfonic anhydride (Tf₂O) (2.9 mL, 16.25 mmol, 1.5 eq) was added to the reaction dropwise and the resulting mixture was stirred at 0° C. for 1 hour. The reaction was diluted with DCM (40 mL) and washed with saturated NaHCO₃ (10 mL), saturated NH₄Cl (10 mL), water and brine. The organic layer was dried over MgSO₄, filtered, and concentrated to give a dark brown oil. The dark brown oil was purified by column chromatography on silica gel (KM3 SCIENTIFIC CORP., Particle size 45-75 μm) eluting with 10% ethyl acetate in hexane to give the title compound as pure product I-a4 (3.68 g, 86%)

To a stirred solution of 1-methyl-6-trifluoromethanesulfonyloxy-1H-indole-2,3-dicarboxylic acid dimethyl ester I-a4 (0.77 g, 1.94 mmol, 1.0 eq) in toluene (20 mL), tert-butyl piperazine-1-carboxylate (1.08 g, 5.82 mmol, 3.0 eq), XPhos (0.18 g, 20 mol %) and Cs₂CO₃ (1.9 g, 5.82 mmol, 3 eq) were added. The mixture was degassed with N₂ for 10 minutes and Pd(OAc)₂ (0.087 g, 20 mol %) was added to mixture. The resulting mixture was stirred at 80° C. for 17 h until no starting material left. The reaction was cooled to room temperature, diluted with ethyl acetate (30 mL) and water (10 mL), and the layers were separated. The aqueous layer was extracted with ethyl acetate (30 mL) and the combined organic layers were washed with brine (20 mL), dried over MgSO₄, filtered, and concentrated to give a crude material. The crude material was purified by column chromatography on silica gel (KM3 SCIENTIFIC CORP., Particle size 45-75 μm) eluting with 25% ethyl acetate in hexane to give the title compound as pure product I-a5 (0.72 g, 86%)

The I-a5 (0.65 g, 1.51 mmol) was treated with a solution of NaOH (0.6 g, 15.1 mmol) in EtOH (30 mL) and heated to reflux (85° C. bath temperature). After 4 h, the reaction mixture was cooled to ambient temperature, removed excess EtOH, diluted with ethyl acetate (30 mL) and acidified with 1 M HCl to pH 3 and the layers were separated. The aqueous layer was then extracted with ethyl acetate (30 mL) and the combined organic layers were washed with brine, dried over MgSO₄, filtered and concentrated to afford a white solid. Then the white solid and Ac₂O (5 mL) were mixed and heated to 140° C. After 2 h, a mono-ester was found by liquid chromatography mass spectrometry (LC-MASS), and the reaction mixture was cooled to ambient temperature. Then excess Ac₂O was removed, and then dried toluene (5 mL) was added, mixed and concentrated. The residue I-a6 was thus produced and then used without any further purification.

A solution of 3-aminopipyridine-2,6-dione hydrochloride (0.11 g, 2 eq) and N,N-diisopropylethylamine (DIPEA) (0.23 mL, 4 eq) in tetrahydrofuran (THF) (2 mL) was stirred at room temperature for 30 min, then a solution of the compound I-a6 (0.13 g, 1 eq) in THF (2 mL) was added. The reaction was stirred at room temperature for 50 min, then the mixture was diluted with ethyl acetate (10 mL) and water (10 mL). 1N HCl_{(aq)} was then added until the pH value of aqueous layer was less than 2 (<2). The aqueous layer was extracted with ethyl acetate (2×10 mL), and the organic layers were combined, washed with brine (10 mL), and dried with MgSO₄. The solvent was removed to give a crude material, and the crude material was used in the next step without purification. The above crude material was dissolved in THF (4 mL), and carbonyldiimidazole (CDI) (0.11 g, 2 eq) and 4-dimethylaminopyridine (DMAP) (4 mg, 0.1 eq) were added. The reaction mixture was stirred at 50° C. for 2 h. After cooling down, the reaction mixture was diluted with ethyl acetate (10 mL) and water (10 mL). 1N HCl_{(aq)} was then added until the pH value of aqueous layer was less than 2 (<2), and the aqueous layer was then extracted with ethyl acetate (2×10 mL). The organic layers were collected and combined, washed with brine (10 mL), and dried over Na₂SO₄. The solvent was removed to give a crude product, and the crude product was purified by flash column chromatography with an eluent (hexane/EtOAc=1/1 to 1/2) to afford a compound I-a7 (0.28 g, 85%). Then the solid I-a7 and CF₃COOH (TFA) (1 ml) were combined and stirred in CH₂Cl₂ (4 mL) at o room temperature for 4 h. After reaction completed, the excess CF₃COOH was removed under vacuum to give a residue. Then the residue was diluted with DCM (10 mL) and basified with Na₂CO₃ to pH 10 and the organic layer was washed with brine (5 mL), dried over Na₂SO₄, filtered and concentrated to afford an intermediate I-A, which was directly used without any purification.

### (2) Preparation of Intermediate I-B

The intermediate I-B was prepared from commercially available benzonitrile, 4-[(trans-3-amino-2,2,4,4-tetramethylcyclobutyl)oxy]-2-chloro-, hydrochloride via the route shown below:

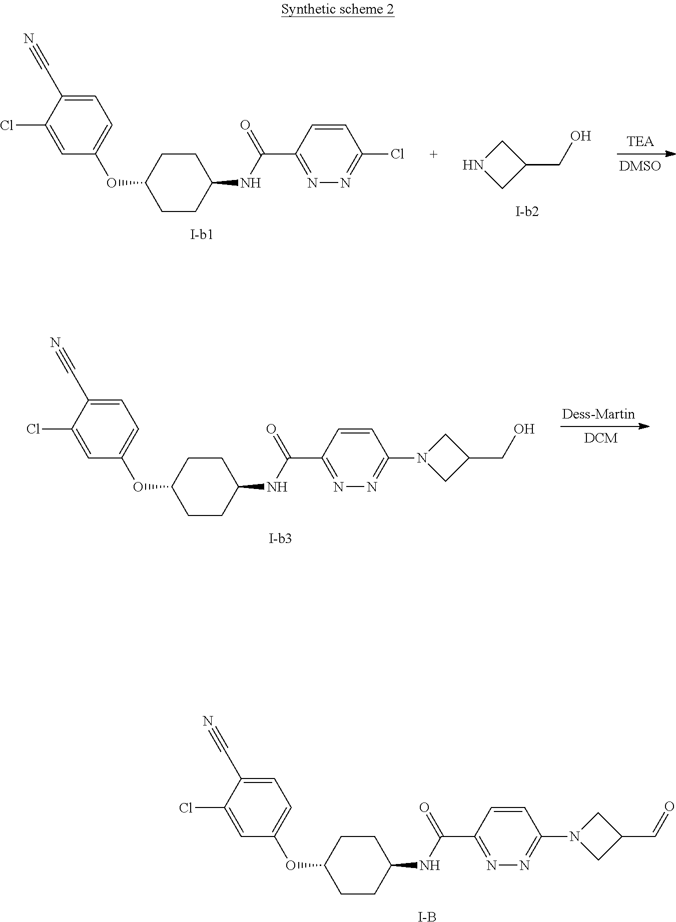

A mixture of 6-Chloro-N-(trans-4-(3-chloro-4-cyanophenoxy) cyclohexyl) pyridazine-3-carboxamide I-b1 (800 mg, 1 eq), compound I-b2 (200 mg, 1.1 eq), and (triethyl) amine (TEA) (0.57 mL, 2 eq) in dimethyl sulfoxide (DMSO) (6 mL) was stirred at 100° C. for 24 h. After cooling down, the mixture was added slowly into water (30 mL). The suspension was filtered to afford compound I-b3 (0.85 g, 94%).

To a solution of I-b3 (0.60 g, 1.36 mmol) in DCM (25 mL) and Dess-Martin periodinane (0.75 g, 1.78 mmol) were added at room temperature then stirred at room temperature for 2 h. The reaction solution was filtered, and the filtrate was washed with saturated Na₂S₂O₃ (10 mL×2), saturated NaHCO₃ (10 mL), brine (10 mL), dried over Na₂SO₄, and concentrated, to provide intermediate I-B (0.54 g, 90%).

(3) Preparation of Compound 1 from Intermediate I-A and Intermediate I-B

Compound 1 was prepared via the route shown below:

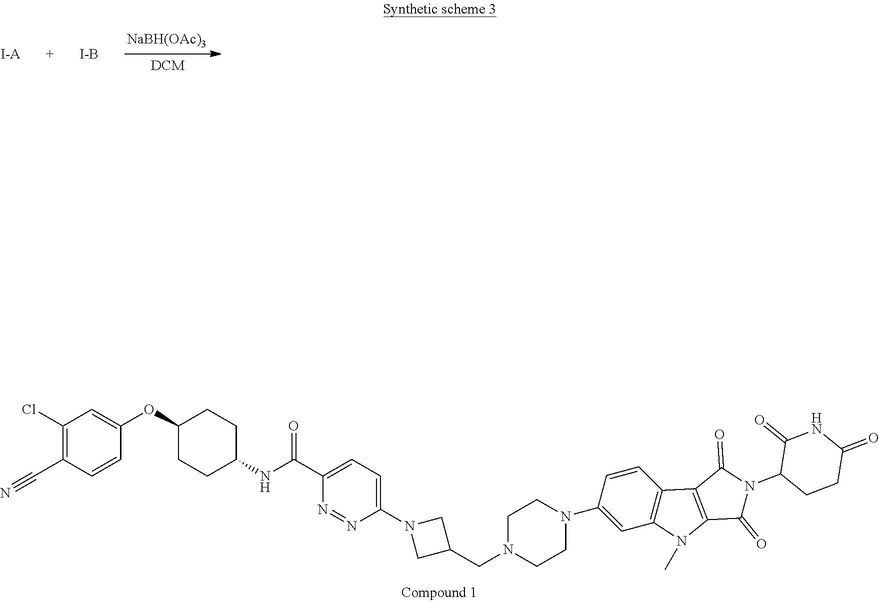 [SMILES: "I-A" ; "I-B" ; Cn1c2c(c3ccc(N4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O ; "NaBH(OAc)3"]

To a mixture of the intermediate I-A (100 mg, 0.25 mmol) and I-B (111 mg, 0.25 mmol) in DCM (1 mL), NaBH(OAc)₃ (107 mg, 0.50 mmol) was added. The resulting solution was stirred for 17 h at room temperature. After reaction was completed, water was added to give a solution, and the solution was diluted with DCM (10 mL). The organic layer was washed with brine (5 mL), dried over Na₂SO₄, filtered and concentrated to afford a crude product. The crude product was purified by column chromatography on silica gel (KM3 SCIENTIFIC CORP., Particle size 45-75 μm) to give Compound 1 (160 mg, 78%). MS: m/z 819.5 (M⁺+1); ¹H NMR (DMSO-d₆) δ 11.05 (s, 1H), 8.55 (d, 1H), 7.87-7.82 (m, 2H), 7.56 (d, 1H), 7.39 (d, 1H), 7.17-7.12 (m, 2H), 7.07 (s 1H), 6.87 (d, 1H), 4.96 (dd, 1H), 4.56-4.51 (m, 1H), 4.26-4.24 (m, 2H), 3.93 (s, 3H), 3.88-3.82 (m, 3H), 3.29 (bs, 4H), 3.13-3.08 (m, 1H), 2.92-2.86 (m, 1H), 2.72-2.70 (m, 2H), 2.59 (bs, 4H), 2.60-2.49 (m, 2H), 2.12-2.09 (m, 2H), 2.05-2.01 (m, 1H), 1.92-1.89 (m, 2H), 1.67-1.61 (m, 2H), 1.54-1.49 (m, 2H).

Compound 1 also could be prepared via the route shown below:

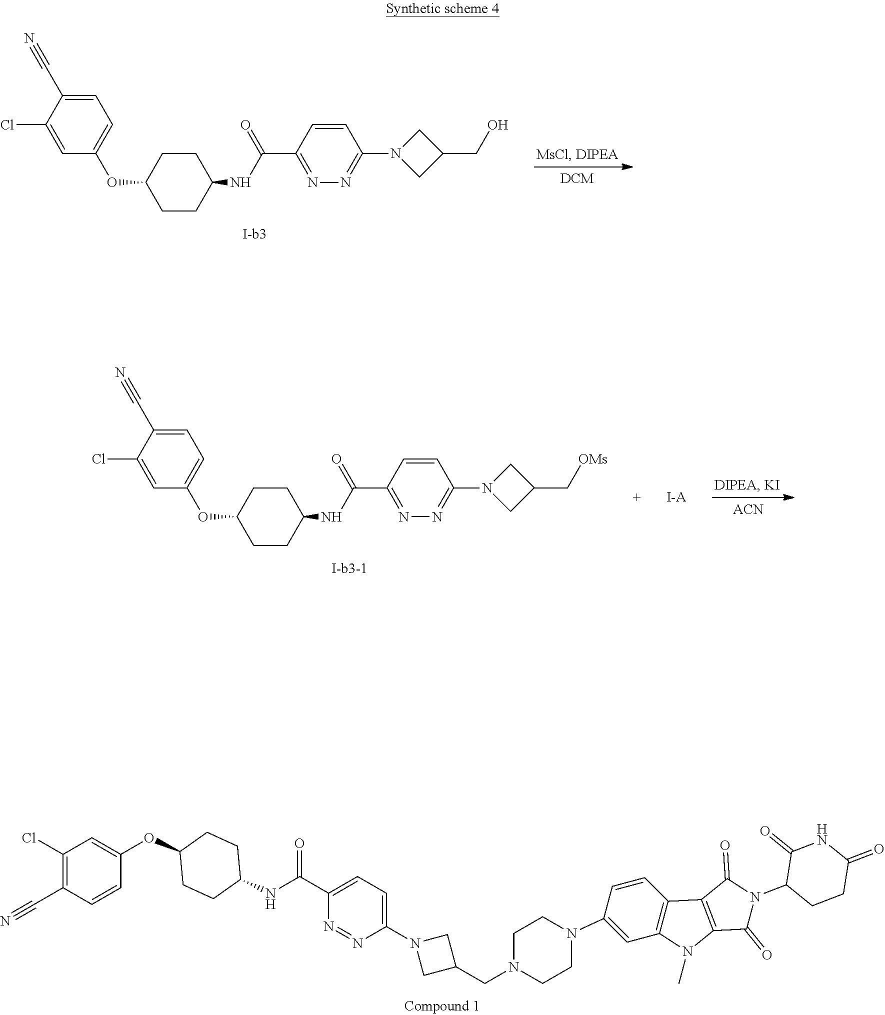 [SMILES: N#Cc1ccc(O[C@H]2CC[C@H](NC(=O)c3ccc(N4CC(CO)C4)nn3)CC2)cc1Cl ; CS(=O)(=O)OCC1CN(c2ccc(C(=O)N[C@H]3CC[C@H](Oc4ccc(C#N)c(Cl)c4)CC3)nn2)C1 ; "I-A" ; "ACN" ; Cn1c2c(c3ccc(N4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O]

To a solution of I-b3 (0.1 g, 0.25 mmol) in DCM (2 mL), DIPEA (0.16 mL, 4.0 eq) was added at room temperature. Then methane sulfonyl chloride (MsCl) (0.038 mL, 2.0 eq) was added dropwise. The mixture reaction was stirred at room temperature for 1 h to 2 h. After the reaction completed, diluted with DCM (10 mL) and washed with saturated NaHCO₃ (5 mL), saturated NH₄Cl (5 mL), and bine (5 mL), dried over Na₂SO₄ (s), concentrated, and purified by column chromatography on silica gel (KM3 SCIENTIFIC CORP., Particle size 45-75 μm) (DCM/acetone=10/1), to afford intermediate I-b3-1 (0.11 g, 84%).

To a solution of I-b3-1 (0.104 g, 0.20 mmol), I-A (0.079 g, 0.20 mmol), KI (0.1 g, 3.0 eq) and DIPEA (0.17 mL, 5.0 eq) in dry CH₃CN (ACN) (2 mL) was heated to 75° C. for 17 h. After the reaction completed, the reaction mixture was diluted with ethyl acetate (10 mL) and basified with saturated Na₂CO₃ to pH 10. The organic layer was washed with brine (5 mL), dried over Na₂SO₄ (s), concentrated and purified by column chromatography on silica gel (KM3 SCIENTIFIC CORP., Particle size 45-75 μm) to get Compound 1 (0.095 g, 58%).

### Synthetic Method B—Synthesis of Compound 2:

### (1) Preparation of Intermediate I-C

The residue I-C was first prepared from commercially available 4-(4,4,5,5-tetramethyl-[1,3,2]dioxaborolan-2-yl)-3,6-dihydro-2H-pyridine-1-carboxylic acid tert-butyl ester via the route shown below:

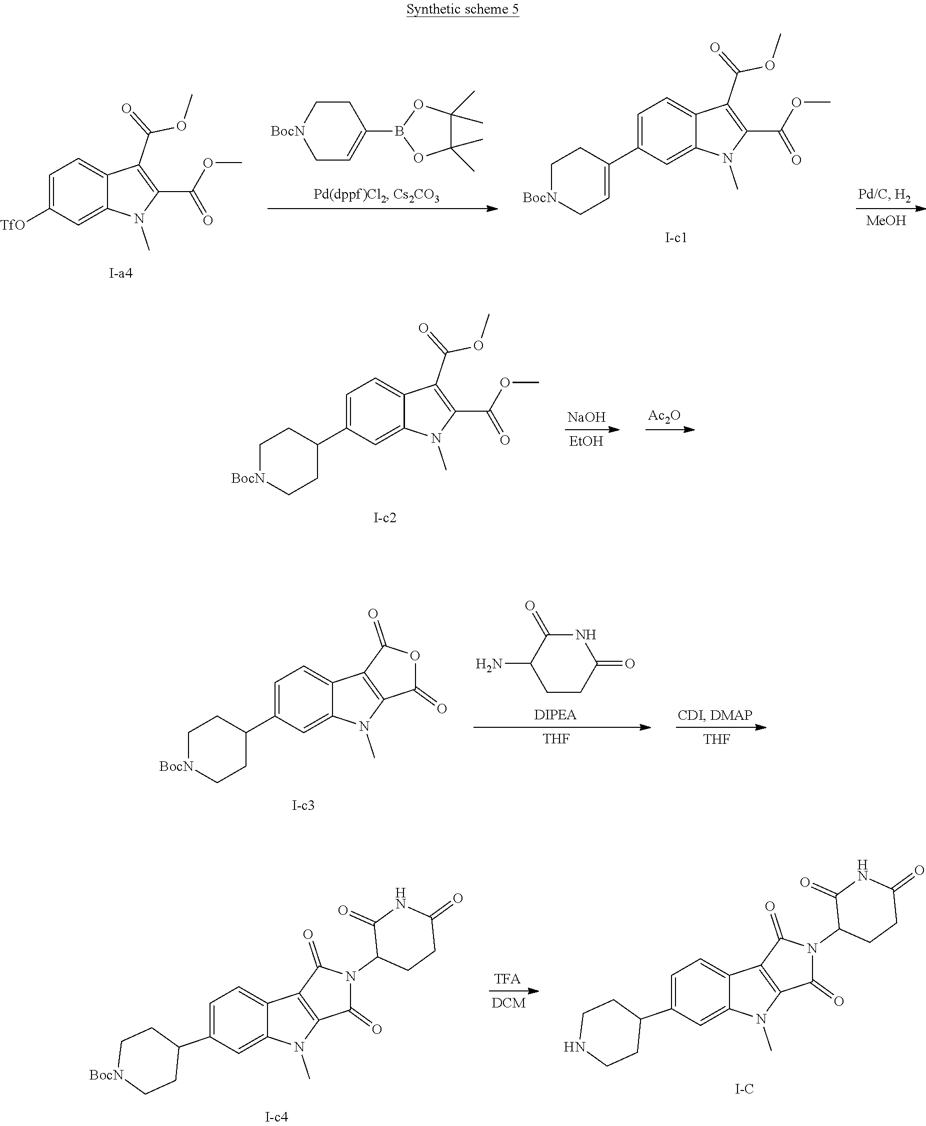

To a stirred solution of 1-methyl-6-trifluoromethanesulfonyloxy-1H-indole-2,3-dicarboxylic acid dimethyl ester I-a4 (683 mg, 1.72 mmol, 1.0 eq) in 1,4-dioxane (17 mL), 4-(4,4,5,5-tetramethyl-[1,3,2]dioxaborolan-2-yl)-3,6-dihydro-2H-pyridine-1-carboxylic acid tert-butyl ester (797 mg, 2.58 mmol, 1.5 eq) and Cs₂CO₃ (1.06 g, 3.26 mmol, 1.9 eq) were added at 25° C. The mixture was degassed with N₂ for 15 minutes and Pd(dppf)Cl₂ (63 mg, 0.09 mmol, 5 mol %) was added at 25° C. The reaction was stirred at 25° C. for 5 h until no starting material left. The reaction solution was diluted with ethyl acetate (30 mL) and water (30 mL). Layers were separated and the aqueous layer was extracted with ethyl acetate (15 mL×2). The combined organic layers were washed with brine, dried over MgSO₄, filtered, and concentrated to give a crude material. The crude material was purified by column chromatography on silica gel (KM3 SCIENTIFIC CORP., Particle size 45-75 μm) eluting with 10% to 15% ethyl acetate in hexane to give intermediate I-c1 (470 mg, 64%, R_{f}=0.21 in EA/Hex=1/5).

To a solution of intermediate I-c1 (2.0 g) in MeOH (20 mL), Pd/C (0.2 g) was added and stirred under hydrogen atmosphere for 2 h. After the reaction was completed, the mixture was passed through a Celite pad and rinsed with EtOAc (10 mL), and the organic solvent was collected and removed on a rotor to afford an intermediate I-c2 (2.0 g, 100%).

The intermediate I-c2 (0.65 g, 1.51 mmol) was treated with a solution of NaOH (0.6 g, 15.1 mmol) in EtOH (30 mL) and heated to reflux. After 4 h, the reaction mixture was cooled to ambient temperature, removed excess EtOH, diluted with ethyl acetate (30 mL) and acidified with 1 M HCl to pH 3. The aqueous layer was then extracted with ethyl acetate (30 mL) and the organic layers were combined, washed with brine, dried (with MgSO₄), filtered and concentrated to afford a white solid. Then the white solid and Ac₂O (5 mL) were combined and heated to 140° C. After 2 h, a mono-ester was found by LC-MASS, and the reaction mixture was cooled to ambient temperature. Then excess Ac₂O was removed, and then dried toluene (5 mL) was added and concentrated. The intermediate I-c3 was thus produced and then used without any further purification.

A solution of 3-aminopiperidine-2,6-dione hydrochloride (0.22 g, 2 eq) and N,N-diisopropylethylamine (DIPEA) (0.46 mL, 4 eq) in THF (2 mL) was stirred at room temperature for 30 min, then a solution of the intermediate I-c3 (0.26 g, 1 eq) in THF (2 mL) was added. The reaction was stirred at room temperature for 50 min, then the mixture was diluted with ethyl acetate (20 mL) and water (20 mL). 1N HCl_{(aq)} was then added until the pH value of aqueous layer was less than 2 (<2). The aqueous layer was extracted with ethyl acetate (2×20 mL), and the organic layers were combined, washed with brine (20 mL), and dried with MgSO₄. The solvent was removed to give a crude material, and the crude material was used in the next step without purification. The above crude material was dissolved in THF (8 mL), and CDI (0.22 g, 2 eq) and DMAP (8 mg, 0.1 eq) were added. The reaction mixture was stirred at 50° C. for 2 h. After cooling down, the reaction mixture was diluted with ethyl acetate (20 mL) and water (20 mL). 1N HCl_{(aq)} was then added until the pH value of aqueous layer was less than 2, and the aqueous layer was then extracted with ethyl acetate (2×20 mL). The organic layers were collected and combined, washed with brine (20 mL), and dried over Na₂SO₄. The solvent was removed to give a crude product, and the crude product was purified by flash column chromatography with an eluent (hexane/EtOAc=1/1 to 1/2) to afford an intermediate I-c4 (0.26 g, 80%). Then the intermediate I-c4 and CF₃COOH (2.5 ml) were combined and stirred in DCM (10 mL) at room temperature for 4 h. After reaction completed, the excess CF₃COOH was removed under vacuum to give a residue. Then the residue was diluted with DCM (10 mL) and basified with Na₂CO₃ to pH 10, and the organic layer was washed with brine (5 mL), dried over Na₂SO₄, filtered and concentrated to afford an intermediate I-C, which was directly used without any purification.

(2) Preparation of Compound 2 from Intermediate I-B and Intermediate I-C

Compound 2 was prepared via the route shown below:

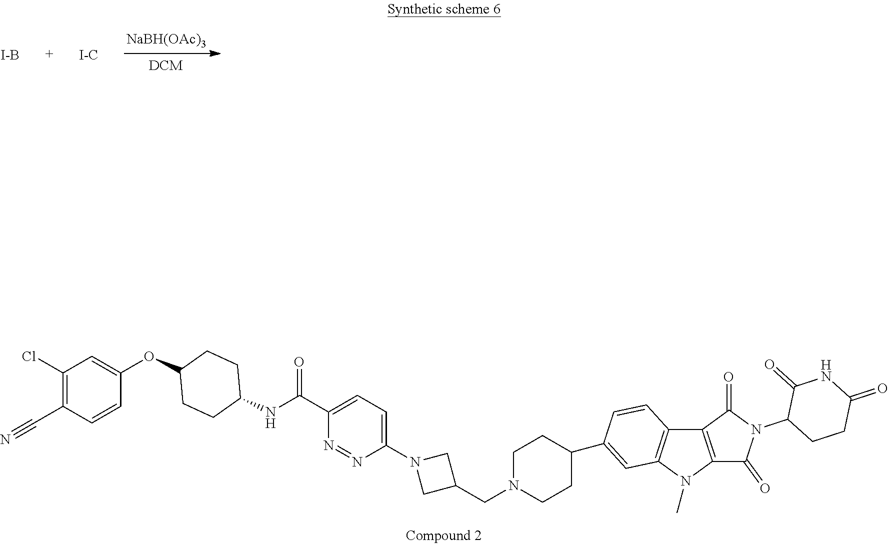

To a mixture of the intermediate I-B (112 mg, 0.254 mmol) and I-C (100 mg, 0.254 mmol) in DCM (1 mL), NaBH(OAc)₃ (107 mg, 0.508 mmol) was added. The resulting solution was stirred for 17 h at room temperature. After reaction was completed, water was added to give a solution, and the solution was diluted with DCM (10 mL). The organic layer was washed with brine (5 mL), dried over Na₂SO₄, filtered and concentrated to afford a crude product. The crude product was purified by column chromatography on silica gel (KM3 SCIENTIFIC CORP., Particle size 45-75 μm) to give Compound 2 (156 mg, 75%). MS: 841.5 (M⁺+23); ¹H NMR (DMSO-d₆) δ 11.07 (s, 1H), 8.56 (d, 1H), 7.87-7.83 (m, 2H), 7.69-7.65 (m, 2H), 7.39 (d, 1H), 7.31 (d, 1H), 7.14 (dd, 1H), 6.87 (d, 1H), 5.00 (dd, 1H), 4.57-4.52 (m, 1H), 4.24 (bs, 2H), 4.00 (s, 3H), 3.89-3.81 (m, 3H), 3.12-3.05 (m, 1H), 3.04-2.98 (m, 1H), 2.93-2.87 (m, 1H), 2.73-2.64 (m, 2H), 2.63-2.50 (m, 6H), 2.13-2.07 (m, 2H), 2.07-2.01 (m, 1H), 1.94-1.88 (m, 2H), 1.87-1.74 (m, 4H), 1.68-1.62 (m, 2H), 1.55-1.49 (m, 2H).

### Synthetic Method C—Synthesis of Chiral Intermediate I-D:

The intermediate I-D was prepared from I-a6 via the route shown below:

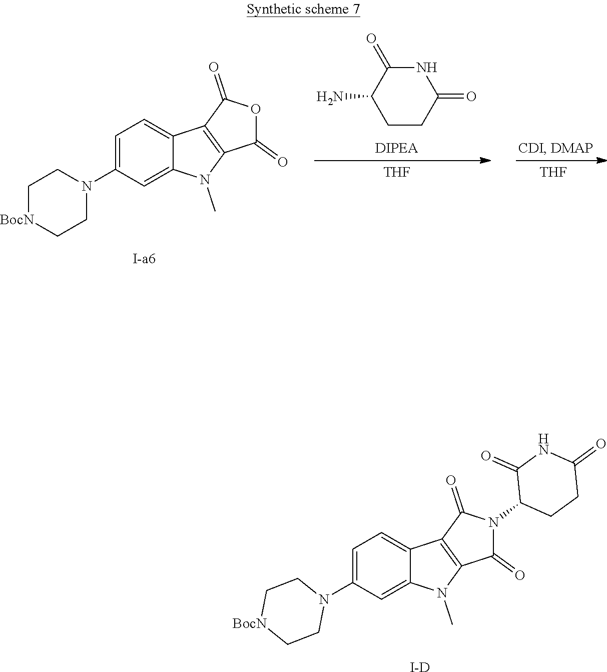

A solution of (S) 3-aminopiperidine-2,6-dione hydrochloride (0.11 g, 2 eq) and N,N-diisopropylethylamine (DIPEA) (0.23 mL, 4 eq) in THF (2 mL) was stirred at room temperature for 30 min, then a solution of the compound I-a6 (0.13 g, 1 eq) in THF (2 mL) was added. The reaction was stirred at room temperature for 50 min, then the mixture was diluted with ethyl acetate (10 mL) and water (10 mL). 1N HCl_{(aq)} was then added until the pH value of aqueous layer was less than 2 (<2). The aqueous layer was extracted with ethyl acetate (2×10 mL), and the organic layers were combined, washed with brine (10 mL), and dried with MgSO₄. The solvent was removed to give a crude material, and the crude material was used in the next step without purification. The above crude material was dissolved in THF (4 mL), and CDI (0.11 g, 2 eq) and

DMAP (4 mg, 0.1 eq) were added. The reaction mixture was stirred at 50° C. for 2 h. After cooling down, the reaction mixture was diluted with ethyl acetate (10 mL) and water (10 mL). 1N HCl_{(aq)} was then added until the pH value of aqueous layer was less than 2 (<2), and the aqueous layer was then extracted with ethyl acetate (2×10 mL). The organic layers were collected, washed with brine (10 mL), and dried over Na₂SO₄. The solvent was removed to give a crude product, and the crude product was purified by flash column chromatography with an eluent (Hexane/EtOAc=1/1 to 1/2) to afford a chiral intermediate I-D (0.15 g, 90%). The chiral intermediate I-D can be used to replace I-A or I-C to give a chiral compound of the present invention.

The preparation of Compounds 1 and 2 of the present invention is exemplified above. Compounds 3-51 in the present invention can be synthesized by similar methods shown in Synthetic Method A (Synthetic schemes 1 to 4), Synthetic Method B (Synthetic schemes 5 to 6) or Synthetic Method C or by any known synthesis methods based on the general knowledge of organic chemistry, with changing one or more starting materials to obtain the desired products.

Compound 3: MS: m/z 855.6 (M⁺+23); ¹H NMR (DMSO-d₆) δ 11.05 (s, 1H), 8.55 (d, 1H), 7.87-7.82 (m, 2H), 7.56 (d, 1H), 7.39 (d, 1H), 7.17-7.12 (m, 3H), 6.87 (d, 1H), 4.96 (dd, 1H), 4.56-4.51 (m, 1H), 4.41 (q, 2H), 4.26-4.24 (m, 2H), 3.89-3.82 (m, 3H), 3.29 (bs, 4H), 3.13-3.08 (m, 1H), 2.92-2.86 (m, 1H), 2.72-2.70 (m, 2H), 2.60 (bs, 4H), 2.60-2.49 (m, 2H), 2.12-2.09 (m, 2H), 2.06-2.03 (m, 1H), 1.92-1.89 (m, 2H), 1.67-1.61 (m, 2H), 1.55-1.49 (m, 2H), 1.41 (t, 3H).

Compound 4: MS: m/z 882.6 (M⁺+23); ¹H NMR (DMSO-d₆) δ 11.04 (s, 1H), 8.57 (m, 1H), 7.98-7.93 (m, 2H), 7.85 (m, 1H), 7.56 (m, 1H), 7.39 (m, 1H), 7.16-7.10 (m, 3H), 6.44 (m, 1H), 4.98-4.95 (m, 1H), 4.54 (m, 1H), 4.40 (m, 2H), 3.79 (m, 1H), 3.71 (m, 1H), 3.58 (m, 1H), 3.33-3.26 (m, 5H), 3.03 (m, 1H), 2.91-2.86 (m, 1H), 2.64-2.50 (m, 5H), 2.45-2.39 (m, 1H), 2.30 (m, 1H), 2.16-2.04 (m, 4H), 1.90 (m, 2H), 1.66 (m, 3H), 1.54-1.46 (m, 4H), 1.40 (m, 3H), 1.24 (m, 2H).

Compound 5: MS: m/z 882.9 (M⁺+23); ¹H NMR (DMSO-d₆) δ 11.05 (s, 1H), 8.59 (d, 1H), 8.02 (d, 1H), 7.94 (dd, 1H), 7.86 (d, 1H), 7.56 (d, 1H), 7.39 (m, 1H), 7.16-7.14 (m, 2H), 7.12 (s, 1H), 6.83 (d, 1H), 4.96 (dd, 1H), 4.57-4.53 (m, 1H), 4.43-4.38 (m, 4H), 3.84-3.78 (m, 1H), 3.28 (bs, 4H), 2.92-2.86 (m, 3H), 2.62-2.58 (m, 1H), 2.55 (bs, 4H), 2.52-2.49 (m, 1H), 2.23-2.22 (m, 2H), 2.12-2.10 (m, 2H), 2.07-2.02 (m, 1H), 1.94-1.85 (m, 3H), 1.85-1.80 (m, 2H), 1.56-1.46 (m, 4H), 1.41 (t, 3H), 1.12-1.07 (m, 2H).

Compound 6: MS: m/z 910.7 (M⁺+23); ¹H NMR (DMSO-d₆) δ 11.05 (s, 1H), 8.62 (d, 1H), 7.94 (dd, 1H), 7.91 (d, 1H), 7.57-7.54 (m, 2H), 7.21 (d, 1H), 7.16 (d, 1H), 7.12 (s, 1H), 7.00 (dd, 1H), 6.47 (d, 1H), 4.96 (dd, 1H), 4.41 (q, 2H), 4.31 (s, 1H), 4.06 (d, 1H), 3.73 (m, 1H), 3.63-3.57 (m, 1H), 3.33-3.29 (m, 1H), 3.29 (bs, 4H), 3.07-3.04 (m, 2H), 2.92-2.85 (m, 1H), 2.62-2.54 (m, 5H), 2.47-2.41 (m, 1H), 2.34-2.28 (m, 1H), 2.19-2.14 (m, 1H), 2.07-2.02 (m, 1H), 1.71-1.64 (m, 3H), 1.41 (t, 3H), 1.28-1.21 (m, 2H), 1.22 (s, 6H), 1.12 (s, 6H).

Compound 7: MS: m/z 910.7 (M⁺+23); ¹H NMR (DMSO-d₆) δ 11.05 (s, 1H), 8.63 (d, 1H), 7.94 (d, 1H), 7.91 (d, 1H), 7.60-7.56 (m, 2H), 7.22 (d, 1H), 7.16 (d, 1H), 7.12 (s, 1H), 7.01 (dd, 1H), 6.86 (d, 1H), 4.96 (dd, 1H), 4.44-4.40 (m, 4H), 4.31 (s, 1H), 4.06 (d, 1H), 3.28 (bs, 4H), 3.08-3.04 (m, 2H), 2.92-2.86 (m, 3H), 2.62-2.60 (m, 1H), 2.55 (bs, 4H), 2.53-2.49 (m, 1H), 2.26-2.21 (m, 2H), 2.07-2.01 (m, 1H), 1.94-1.87 (m, 1H), 1.84-1.82 (m, 2H), 1.41 (t, 3H), 1.28-1.21 (m, 2H), 1.22 (s, 6H), 1.12 (s, 6H).

Compound 8: MS: m/z 912.0 (M⁺+23); ¹H NMR (DMSO-d₆) δ 11.05 (s, 1H), 8.22 (d, 1H), 7.91 (d, 1H), 7.84 (d, 1H), 7.56 (d, 1H), 7.26 (d, 1H), 7.17 (d, 1H), 7.12 (s, 1H), 7.04 (dd, 1H), 6.98 (d, 1H), 4.96 (dd, 1H), 4.46 (s, 1H), 4.41 (q, 2H), 4.01 (d, 1H), 3.89-3.64 (m, 2H), 3.32-3.24 (m, 6H), 2.93-2.86 (m, 1H), 2.65-2.55 (m, 5H), 2.47-2.42 (m, 1H), 2.40-2.33 (m, 1H), 2.24-2.18 (m, 1H), 2.07-2.02 (m, 1H), 1.74-1.67 (m, 3H), 1.41 (t, 3H), 1.29-1.21 (m, 2H), 1.22 (s, 6H), 1.15 (s, 6H).

Compound 9: MS: m/z 889.9 (M′+1); ¹H NMR (DMSO-d₆) δ 11.05 (s, 1H), 8.24 (d, 1H), 7.91 (d, 1H), 7.83 (d, 1H), 7.56 (d, 1H), 7.38 (d, 1H), 7.26 (d, 1H), 7.16 (d, 1H), 7.12 (s, 1H), 7.04 (dd, 1H), 4.96 (dd, 1H), 4.53-4.50 (m, 2H), 4.47 (s, 1H), 4.42-4.39 (m, 2H), 4.01 (d, 1H), 3.28 (bs, 4H), 3.08-3.04 (m, 2H), 2.92-2.86 (m, 1H), 2.62-2.60 (m, 1H), 2.56 (bs, 4H), 2.53-2.48 (m, 1H), 2.26-2.24 (m, 2H), 2.08-2.02 (m, 1H), 2.00-1.93 (m, 1H), 1.88-1.86 (m, 2H), 1.41 (t, 3H), 1.22 (s, 6H), 1.14 (s, 6H), 1.20-1.12 (m, 2H).

Compound 10: MS: m/z 854.8 (M⁺+23); ¹H NMR (DMSO-d₆) δ 11.07 (s, 1H), 8.55 (d, 1H), 7.87-7.83 (m, 2H), 7.72 (s, 1H), 7.67 (d, 1H), 7.39 (d, 1H), 7.30 (d, 1H), 7.14 (dd, 1H), 6.87 (d, 1H), 5.00 (dd, 1H), 4.57-4.52 (m, 1H), 4.48 (q, 2H), 4.26-4.23 (m, 2H), 3.89-3.82 (m, 3H), 3.12-3.05 (m, 1H), 3.04-2.98 (m, 1H), 2.93-2.87 (m, 1H), 2.73-2.64 (m, 3H), 2.63-2.50 (m, 4H), 2.15-2.04 (m, 3H), 1.94-1.88 (m, 2H), 1.87-1.74 (m, 4H), 1.67-1.62 (m, 2H), 1.55-1.49 (m, 2H), 1.44 (t, 3H).

Compound 11: MS: m/z 860.9 (M⁺+1); ¹H NMR (DMSO-d₆) δ 11.07 (s, 1H), 8.59 (d, 1H), 7.86 (d, 1H), 7.82 (d, 1H), 7.71-7.66 (m, 2H), 7.39 (d, 1H), 7.34 (d, 1H), 7.30 (d, 1H), 7.14 (dd, 1H), 5.00 (dd, 1H), 4.56-4.46 (m, 5H), 3.89-3.83 (m, 1H), 3.06-2.95 (m, 3H), 2.93-2.86 (m, 1H), 2.72-2.64 (m, 1H), 2.63-2.56 (m, 1H), 2.55-2.50 (m, 4H), 2.23-2.17 (m, 2H), 2.12-2.09 (m, 2H), 2.08-1.98 (m, 3H), 1.92-1.76 (m, 7H), 1.67-1.62 (m, 2H), 1.55-1.49 (m, 2H), 1.44 (t, 3H), 1.19-1.10 (m, 2H).

Compound 12: MS: m/z 861.0 (M⁺+1); ¹H NMR (DMSO-d₆) δ 11.07 (s, 1H), 8.52 (d, 1H), 7.87-7.83 (m, 3H), 7.74-7.65 (m, 2H), 7.39 (d, 1H), 7.30 (d, 1H), 7.14 (dd, 1H), 6.95 (d, 1H), 5.00 (dd, 1H), 4.60-4.52 (m, 1H), 4.48 (q, 2H), 3.89-3.83 (m, 1H), 3.82-3.62 (m, 2H), 3.22-3.10 (m, 1H), 3.10-2.96 (m, 2H), 2.93-2.86 (m, 1H), 2.64-2.59 (m, 1H), 2.58-2.50 (m, 5H), 2.42-2.33 (m, 1H), 2.26-2.16 (m, 1H), 2.14-2.09 (m, 2H), 2.08-1.98 (m, 3H), 1.92-1.88 (m, 2H), 1.88-1.75 (m, 4H), 1.75-1.61 (m, 5H), 1.56-1.49 (m, 2H), 1.44 (t, 3H), 1.29-1.24 (m, 2H).

Compound 13: MS: m/z 886.8 (M⁺+1)

Compound 14: MS: m/z 900.8 (M⁺+1);

Compound 15: MS: m/z 909.5 (M⁺+23); ¹H NMR (DMSO-d₆) δ 11.05 (s, 1H), 7.91 (d, 1H), 7.75 (d, 1H), 7.56 (d, 1H), 7.38 (d, 1H), 7.21 (d, 1H), 7.17 (d, 1H), 7.12 (s, 1H), 7.01 (dd, 1H), 6.54 (d, 1H), 4.96 (dd, 1H), 4.46 (s, 1H), 4.41 (q, 2H), 4.32 (s, 2H), 4.06 (d, 1H), 3.54-3.51 (m, 1H), 3.41-3.37 (m, 2H), 3.29 (bs, 4H), 2.97-2.94 (m, 1H), 2.92-2.85 (m, 1H), 2.61-2.54 (m, 5H), 2.47-2.42 (m, 1H), 2.35-2.31 (m, 1H), 2.24-2.18 (m, 1H), 2.19-2.17 (m, 1H), 2.06-2.03 (m, 1H), 1.70-1.65 (m, 3H), 1.41 (t, 3H), 1.28-1.23 (m, 2H), 1.22 (s, 6H), 1.13 (s, 6H).

Compound 16: MS: m/z 888.0 (M⁺+1)

Compound 17: MS: m/z 847.9 (M⁺+1); ¹H NMR (DMSO-d₆) δ 11.05 (s, 1H), 8.52 (d, 1H), 7.86 (d, 1H), 7.82 (d, 1H), 7.56 (d, 1H), 7.39 (d, 1H), 7.17-7.12 (m, 3H), 6.96 (d, 1H), 4.96 (dd, 1H), 4.57-4.52 (m, 1H), 4.41 (q, 2H), 3.89-3.83 (m, 1H), 3.80-3.60 (m, 2H), 3.57-3.45 (m, 1H), 3.32-3.26 (m, 5H), 2.92-2.86 (m, 1H), 2.71-2.55 (m, 5H), 2.47-2.45 (m, 2H), 2.19-2.14 (m, 1H), 2.12-2.09 (m, 2H), 2.06-2.02 (m, 1H), 1.92-1.91 (m, 2H), 1.82-1.76 (m, 1H), 1.67-1.61 (m, 2H), 1.55-1.49 (m, 2H), 1.41 (t, 3H), 1.29-1.24 (m, 2H).

Compound 18: MS: m/z 846.6 (M⁺+1); ¹H NMR (DMSO-d₆) δ 11.07 (s, 1H), 8.51 (d, 1H), 7.86 (d, 1H), 7.83 (d, 1H), 7.72 (s, 1H), 7.67 (d, 1H), 7.40 (d, 1H), 7.31 (d, 1H), 7.14 (dd, 1H), 6.96 (d, 1H), 5.00 (dd, 1H), 4.57-4.53 (m, 1H), 4.49 (q, 2H), 3.89-3.83 (m, 1H), 3.79-3.57 (m, 2H), 3.56-3.46 (m, 1H), 3.14-3.07 (m, 1H), 3.06-3.00 (m, 1H), 2.93-2.87 (m, 1H), 2.72-2.63 (m, 2H), 2.63-2.59 (m, 1H), 2.58-2.53 (m, 3H), 2.44-2.40 (m, 2H), 2.19-2.14 (m, 1H), 2.13-2.10 (m, 2H), 2.08-2.04 (m, 2H), 1.93-1.90 (m, 2H), 1.87-1.76 (m, 5H), 1.67-1.62 (m, 2H), 1.55-1.49 (m, 2H), 1.44 (t, 3H), 1.29-1.24 (m, 2H).

Compound 19: MS: m/z 887.4 (M⁺+1).

Compound 20: MS: m/z 901.5 (M⁺+1).

Compound 21: MS: m/z 869.7 (M⁺+23).

Compound 22: MS: m/z 869.3 (M⁺+23); ¹H NMR (DMSO-d₆) δ 11.05 (s, 1H), 8.52 (d, 1H), 7.86 (d, 1H), 7.82 (d, 1H), 7.56 (d, 1H), 7.39 (d, 1H), 7.17 (d, 1H), 7.14 (dd, 1H), 7.07 (bs, 1H), 6.94 (d, 1H), 4.96 (dd, 1H), 4.56-4.51 (m, 1H), 3.93 (s, 3H), 3.88-3.83 (m, 1H), 3.50-3.42 (m, 2H), 3.42-3.30 (m, 1H), 3.29 (bs, 4H), 3.18-3.11 (m, 1H), 2.92-2.86 (m, 1H), 2.62-2.55 (m, 5H), 2.48-2.41 (m, 1H), 2.40-2.33 (m, 1H), 2.24-2.18 (m, 1H), 2.12-2.09 (m, 2H), 2.06-2.01 (m, 1H), 1.94-1.89 (m, 2H), 1.75-1.61 (m, 5H), 1.55-1.49 (m, 2H), 1.29-1.21 (m, 2H).

Compound 23: MS: m/z 883.8 (M⁺+23); ¹H NMR (DMSO-d₆) δ 11.05 (s, 1H), 8.59 (d, 1H), 7.86 (d, 1H), 7.81 (d, 1H), 7.56 (d, 1H), 7.39 (d, 1H), 7.34 (d, 1H), 7.17-7.12 (m, 3H), 4.96 (dd, 1H), 4.56-4.48 (m, 3H), 4.41 (q, 2H), 3.87-3.86 (m, 1H), 3.28 (bs, 4H), 3.06-3.02 (m, 2H), 2.92-2.86 (m, 1H), 2.62-2.60 (m, 1H), 2.55 (bs, 4H), 2.53-2.48 (m, 1H), 2.26-2.21 (m, 2H), 2.12-2.09 (m, 2H), 2.06-2.02 (m, 1H), 1.97-1.85 (m, 5H), 1.67-1.61 (m, 2H), 1.55-1.49 (m, 2H), 1.41 (t, 3H), 1.18-1.12 (m, 2H).

Compound 24: MS: m/z 908.6 (M⁺+23); ¹H NMR (DMSO-d₆) δ 11.07 (s, 1H), 7.92 (d, 1H), 7.75 (d, 2H), 7.72-7.66 (m, 2H), 7.39 (d, 1H), 7.30 (d, 1H), 7.21 (d, 1H), 7.01 (dd, 1H), 6.55 (d, 1H), 5.00 (dd, 1H), 4.47 (q, 2H), 4.33 (s, 1H), 4.07 (d, 1H), 3.54-3.51 (m, 1H), 3.43-3.34 (m, 2H), 3.12-3.00 (m, 1H), 2.99-2.94 (m, 1H), 2.93-2.87 (m, 1H), 2.65-2.59 (m, 1H), 2.59-2.49 (m, 5H), 2.36-2.31 (m, 1H), 2.21-2.15 (m, 1H), 2.09-2.02 (m, 2H), 1.94-1.77 (m, 4H), 1.72-1.63 (m, 2H), 1.44 (t, 3H), 1.28-1.22 (m, 2H), 1.22 (s, 6H), 1.13 (s, 6H).

Compound 25: MS: m/z 883.5 (M⁺+23); ¹H NMR (DMSO-d₆) δ 11.05 (s, 1H), 8.75 (bs, 2H), 8.11 (d, 1H), 7.87 (d, 1H), 7.56 (d, 1H), 7.40 (d, 1H), 7.17-7.14 (m, 2H), 7.12 (bs, 1H), 4.96 (dd, 1H), 4.75-4.73 (m, 2H), 4.58-4.53 (m, 1H), 4.42-4.39 (m, 2H), 3.83-3.77 (m, 1H), 3.28 (bs, 4H), 3.00-2.96 (m, 2H), 2.92-2.86 (m, 1H), 2.62-2.59 (m, 2H), 2.55 (bs, 4H), 2.24-2.22 (m, 2H), 2.12-2.09 (m, 2H), 2.07-2.01 (m, 1H), 1.94-1.89 (m, 3H), 1.84-1.83 (m, 2H), 1.55-1.47 (m, 4H), 1.41 (t, 3H), 1.10-1.03 (m, 2H).

Compound 26: MS: m/z 883.5 (M⁺+23); ¹H NMR (DMSO-d₆) δ 11.05 (s, 1H), 8.75 (bs, 2H), 8.12 (d, 1H), 7.86 (d, 1H), 7.56 (d, 1H), 7.39 (s, 1H), 7.17-7.12 (m, 3H), 4.96 (dd, 1H), 4.59-4.52 (m, 1H), 4.42-4.39 (m, 2H), 3.86-3.75 (m, 2H), 3.30-3.25 (m, 1H), 3.13-3.10 (m, 1H), 2.92-2.86 (m, 1H), 2.62-2.52 (m, 6H), 2.33-2.67 (m, 1H), 2.19-2.07 (m, 3H), 2.07-2.01 (m, 1H), 1.94-1.87 (m, 2H), 1.72-1.62 (m, 3H), 1.55-1.47 (m, 4H), 1.40 (t, 3H), 1.28-1.23 (m, 2H).

Compound 27: MS: m/z 874.8 (M⁺+1); ¹H NMR (DMSO-d₆) δ 11.05 (s, 1H), 8.62 (bs, 1H), 7.94 (dd, 1H), 7.90 (d, 1H), 7.57-7.54 (m, 2H), 7.21 (d, 1H), 7.17 (d, 1H), 7.06 (s, 1H), 7.01 (dd, 1H), 6.47 (d, 1H), 4.96 (dd, 1H), 4.31 (s, 1H), 4.06 (d, 1H), 3.93 (s, 3H), 3.75-3.68 (m, 1H), 3.63-3.56 (m, 1H), 3.31-3.24 (m, 5H), 3.07-3.04 (m, 1H), 2.92-2.86 (m, 1H), 2.61-2.54 (m, 5H), 2.46-2.42 (m, 1H), 2.34-2.28 (m, 1H), 2.20-2.14 (m, 1H), 2.04-2.00 (m, 1H), 1.69-1.64 (m, 3H), 1.28-1.21 (m, 2H), 1.22 (s, 6H), 1.12 (s, 6H).

Compound 28: MS: m/z 896.7 (M⁺+23); ¹H NMR (DMSO-d₆) δ 11.05 (s, 1H), 8.62 (bs, 1H), 7.94 (d, 1H), 7.91 (d, 1H), 7.60-7.55 (m, 2H), 7.22 (s, 1H), 7.17 (d, 1H), 7.06 (s, 1H), 7.01 (d, 1H), 6.86 (d, 1H), 4.96 (dd, 1H), 4.44-4.40 (m, 2H), 4.31 (s, 1H), 4.06 (d, 1H), 3.93 (s, 3H), 3.35-3.33 (m, 2H), 3.29 (bs, 4H), 2.97-2.86 (m, 3H), 2.61-2.54 (m, 6H), 2.24-2.22 (m, 2H), 2.06-2.00 (m, 1H), 1.94-1.87 (m, 1H), 1.86-1.81 (m, 2H), 1.26-1.22 (m, 2H), 1.22 (s, 6H), 1.13 (s, 6H).

Compound 29: MS: m/z 846.3 (M⁺+1); ¹H NMR (DMSO-d₆) δ 11.07 (s, 1H), 8.59 (d, 1H), 7.86 (d, 1H), 7.81 (d, 1H), 7.67-7.64 (m, 2H), 7.39 (d, 1H), 7.34 (d, 1H), 7.31 (d, 1H), 7.14 (dd, 1H), 5.00 (dd, 1H), 4.57-4.48 (m, 3H), 4.00 (s, 3H), 3.90-3.83 (m, 1H), 3.05-2.98 (m, 3H), 2.93-2.87 (m, 1H), 2.72-2.64 (m, 1H), 2.63-2.56 (m, 1H), 2.55-2.50 (m, 4H), 2.24-2.17 (m, 2H), 2.12-2.09 (m, 2H), 2.07-1.99 (m, 3H), 1.92-1.76 (m, 7H), 1.68-1.62 (m, 2H), 1.55-1.49 (m, 2H), 1.19-1.10 (m, 2H).

Compound 30: MS: m/z 846.6 (M⁺+1); ¹H NMR (DMSO-d₆) δ 11.07 (s, 1H), 8.52 (d, 1H), 7.86 (d, 1H), 7.83 (d, 1H), 7.71-7.65 (m, 1H), 7.63 (bs, 1H), 7.39 (d, 1H), 7.30 (d, 1H), 7.14 (dd, 1H), 6.95 (d, 1H), 5.00 (dd, 1H), 4.57-4.52 (m, 1H), 4.00 (s, 3H), 3.89-3.83 (m, 1H), 3.55-3.40 (m, 2H), 3.20-2.98 (m, 3H), 2.93-2.87 (m, 1H), 2.75-2.64 (m, 1H), 2.63-2.50 (m, 4H), 2.45-2.32 (m, 2H), 2.25-2.17 (m, 1H), 2.12-2.10 (m, 2H), 2.07-1.98 (m, 3H), 1.92-1.88 (m, 3H), 1.88-1.74 (m, 4H), 1.74-1.62 (m, 4H), 1.55-1.49 (m, 2H).

Compound 31: MS: m/z 886.8 (M⁺+1).

Compound 32: MS: m/z 900.8 (M⁺+1).

Compound 33: MS: m/z 884.0 (M⁺+23); ¹H NMR (DMSO-d₆) δ 11.05 (s, 1H), 8.52 (d, 1H), 7.86 (d, 1H), 7.82 (d, 1H), 7.56 (d, 1H), 7.39 (d, 1H), 7.17-7.12 (m, 3H), 6.94 (d, 1H), 4.96 (dd, 1H), 4.56-4.51 (m, 1H), 4.42-4.39 (m, 2H), 3.88-3.83 (m, 1H), 3.31-3.27 (m, 8H), 3.06-3.02 (m, 2H), 2.92-2.86 (m, 1H), 2.62-2.60 (m, 1H), 2.65-2.55 (m, 5H), 2.48-2.43 (m, 1H), 2.37-2.34 (m, 1H), 2.21-2.17 (m, 1H), 2.12-2.09 (m, 2H), 2.06-2.02 (m, 1H), 1.97-1.89 (m, 2H), 1.74-1.61 (m, 5H), 1.55-1.49 (m, 2H), 1.41 (t, 3H), 1.26-1.23 (m, 2H).

Compound 34: MS: m/z 832.4 (M⁺+1); ¹H NMR (DMSO-d₆) δ 11.07 (s, 1H), 8.51 (d, 1H), 7.86 (d, 1H), 7.83 (d, 1H), 7.68-7.65 (m, 2H), 7.40 (d, 1H), 7.32 (d, 1H), 7.14 (dd, 1H), 6.96 (d, 1H), 5.00 (dd, 1H), 4.57-4.53 (m, 1H), 4.00 (s, 3H), 3.89-3.83 (m, 1H), 3.78-3.59 (m, 2H), 3.55-3.45 (m, 1H), 3.14-3.07 (m, 1H), 3.06-2.99 (m, 1H), 2.93-2.87 (m, 1H), 2.74-2.63 (m, 2H), 2.63-2.59 (m, 1H), 2.58-2.53 (m, 3H), 2.46-2.39 (m, 2H), 2.19-2.04 (m, 5H), 1.95-1.88 (m, 2H), 1.87-1.76 (m, 5H), 1.67-1.61 (m, 2H), 1.55-1.49 (m, 2H).

Compound 35: MS: m/z 847.4 (M⁺+1); ¹H NMR (DMSO-d₆) δ 11.05 (s, 1H), 8.75 (s, 2H), 8.11 (d, 1H), 7.87 (d, 1H), 7.56 (d, 1H), 7.40 (d, 1H), 7.17-7.14 (m, 2H), 7.06 (bs, 1H), 4.96 (dd, 1H), 4.75-4.73 (m, 2H), 4.58-4.53 (m, 1H), 3.93 (s, 3H), 3.83-3.77 (m, 1H), 3.28 (bs, 4H), 3.00-2.96 (m, 2H), 2.92-2.86 (m, 1H), 2.62-2.59 (m, 1H), 2.55 (bs, 4H), 2.55-2.49 (m, 1H), 2.24-2.22 (m, 2H), 2.12-2.08 (m, 2H), 2.06-2.00 (m, 1H), 1.95-1.88 (m, 3H), 1.84-1.83 (m, 2H), 1.55-1.46 (m, 4H), 1.10-1.03 (m, 2H).

Compound 36: MS: m/z 861.7 (M⁺+1); ¹H NMR (DMSO-d₆) δ 11.05 (s, 1H), 8.59 (d, 1H), 7.86 (d, 1H), 7.81 (d, 1H), 7.56 (d, 1H), 7.40 (d, 1H), 7.34 (d, 1H), 7.17-7.12 (m, 3H), 4.96 (dd, 1H), 4.57-4.48 (m, 3H), 4.41 (q, 2H), 3.89-3.83 (m, 1H), 3.28 (bs, 4H), 3.06-3.02 (m, 2H), 2.92-2.86 (m, 1H), 2.62-2.60 (m, 1H), 2.58-2.52 (m, 5H), 2.26-2.22 (m, 2H), 2.13-2.09 (m, 2H), 2.06-2.02 (m, 1H), 1.97-1.85 (m, 5H), 1.67-1.62 (m, 2H), 1.55-1.48 (m, 2H), 1.41 (t, 3H), 1.18-1.12 (m, 2H).

Compound 37: MS: m/z 886.8 (M⁺+1).

Compound 38: MS: m/z 900.8 (M⁺+1).

Compound 39: MS: m/z 862.0 (M⁺+1); ¹H NMR (DMSO-d₆) δ 11.05 (s, 1H), 8.52 (d, 1H), 7.86 (d, 1H), 7.82 (d, 1H), 7.56 (d, 1H), 7.39 (d, 1H), 7.17-7.12 (m, 3H), 6.94 (d, 1H), 4.96 (dd, 1H), 4.57-4.52 (m, 1H), 4.41 (q, 2H), 3.88-3.83 (m, 1H), 3.82-3.60 (m, 2H), 3.50-3.41 (m, 1H), 3.31-3.27 (m, 4H), 3.18-3.09 (m, 1H), 2.92-2.86 (m, 1H), 2.62-2.54 (m, 5H), 2.48-2.43 (m, 1H), 2.39-2.32 (m, 1H), 2.23-2.17 (m, 1H), 2.12-2.09 (m, 2H), 2.06-2.02 (m, 1H), 1.93-1.89 (m, 2H), 1.74-1.61 (m, 5H), 1.55-1.49 (m, 2H), 1.41 (t, 3H), 1.29-1.24 (m, 2H).

Compound 40: MS: m/z 860.9 (M⁺+1); ¹H NMR (DMSO-d₆) δ 11.04 (s, 1H), 9.02 (d, 1H), 8.11 (d, 1H), 7.86 (d, 1H), 7.84 (d, 1H), 7.53 (d, 1H), 7.40 (d, 1H), 7.15 (dd, 1H), 6.78 (d, 1H), 6.61 (s, 1H), 4.94 (dd, 1H), 4.57-4.52 (m, 1H), 4.36 (q, 2H), 3.95-3.89 (m, 1H), 3.56-3.55 (m, 1H), 3.46-3.43 (m, 1H), 3.10-2.95 (m, 3H), 2.92-2.85 (m, 1H), 2.62-2.54 (m, 2H), 2.54-2.49 (m, 4H), 2.46-2.40 (m, 1H), 2.38-2.32 (m, 1H), 2.23-2.17 (m, 1H), 2.15-2.00 (m, 5H), 1.97-1.82 (m, 6H), 1.74-1.64 (m, 5H), 1.56-1.50 (m, 2H), 1.40 (t, 3H).

Compound 41: MS: m/z 869.9 (M⁺+23); ¹H NMR (DMSO-d₆) δ 11.06 (s, 1H), 8.59 (d, 1H), 7.86 (d, 1H), 7.81 (d, 1H), 7.63 (d, 1H), 7.40 (d, 1H), 7.34 (d, 1H), 7.28 (dd, 1H), 7.14 (dd, 1H), 7.03 (bs, 1H), 4.98 (dd, 1H), 4.57-4.52 (m, 1H), 4.52-4.46 (m, 2H), 3.96 (s, 3H), 3.90-3.83 (m, 1H), 3.18 (bs, 4H), 3.05-3.01 (m, 2H), 2.93-2.87 (m, 1H), 2.62-2.52 (m, 6H), 2.24-2.22 (m, 2H), 2.12-2.08 (m, 2H), 2.06-2.01 (m, 1H), 1.97-1.88 (m, 3H), 1.87-1.83 (m, 2H), 1.68-1.62 (m, 2H), 1.55-1.49 (m, 2H), 1.18-1.12 (m, 2H).

Compound 42: MS: m/z 869.7 (M⁺+23); ¹H NMR (DMSO-d₆) δ 11.06 (s, 1H), 8.52 (d, 1H), 7.86 (d, 1H), 7.82 (d, 1H), 7.63 (d, 1H), 7.39 (d, 1H), 7.28 (dd, 1H), 7.14 (dd, 1H), 7.03 (bs, 1H), 6.94 (d, 1H), 4.98 (dd, 1H), 4.57-4.52 (m, 1H), 3.96 (s, 3H), 3.89-3.82 (m, 1H), 3.74-3.63 (m, 1H), 3.51-3.60 (m, 2H), 3.19 (bs, 4H), 3.16-3.09 (m, 1H), 2.92-2.86 (m, 1H), 2.62-2.54 (m, 5H), 2.47-2.41 (m, 1H), 2.39-2.32 (m, 1H), 2.22-2.17 (m, 1H), Compound 43: MS: m/z 886.8 (M⁺+1).

Compound 44: MS: m/z 900.6 (M⁺+1).

Compound 45: MS: m/z 861.4 (M⁺+1).

Compound 46: MS: m/z 846.8 (M⁺+1).

Compound 47: MS: m/z 841.6 (M⁺+23); ¹H NMR (DMSO-d₆) δ 11.06 (s, 1H), 8.56 (d, 1H), 7.87-7.82 (m, 2H), 7.63 (d, 1H), 7.39 (d, 1H), 7.29 (dd, 1H), 7.14 (dd, 1H), 7.04 (s, 1H), 6.87 (d, 1H), 4.99 (dd, 1H), 4.56-4.51 (m, 1H), 4.26-4.23 (m, 2H), 3.96 (s, 3H), 3.89-3.82 (m, 3H), 3.19 (bs, 4H), 3.12-3.07 (m, 1H), 2.93-2.87 (m, 1H), 2.72-2.70 (m, 2H), 2.60 (bs, 4H), 2.60-2.49 (m, 1H), 2.11-2.09 (m, 2H), 2.06-2.01 (m, 1H), 1.92-1.89 (m, 2H), 1.67-1.61 (m, 2H), 1.55-1.49 (m, 2H).

Compound 48: MS: m/z 858.5 (M⁺+1).

Compound 49: MS: m/z 858.5 (M⁺+1); ¹H NMR (DMSO-d₆) δ 11.08 (s, 1H), 8.56 (d, 1H), 7.87-7.83 (m, 3H), 7.70 (d, 1H), 7.52 (dd, 1H), 7.39 (d, 1H), 7.14 (dd, 1H), 6.87 (d, 1H), 6.35-6.32 (m, 1H), 5.01 (dd, 1H), 4.56-4.49 (m, 3H), 4.26-4.24 (m, 2H), 3.89-3.83 (m, 3H), 3.20-3.16 (m, 1H), 3.16-3.09 (m, 1H), 2.93-2.87 (m, 1H), 2.79-2.75 (m, 1H), 2.74-2.72 (m, 1H), 2.64-2.60 (m, 2H), 2.59-2.50 (m, 5H), 2.14-2.04 (m, 3H), 1.93-1.88 (m, 2H), 1.67-1.61 (m, 2H), 1.55-1.49 (m, 2H), 1.44 (t, 3H).

Compound 50: MS: m/z 886.8 (M⁺+1).

Compound 51: MS: m/z 902.7 (M⁺+1); ¹H NMR (DMSO-d₆) δ 11.05 (s, 1H), 8.55 (d, 1H), 7.85 (d, 1H), 7.80 (d, 1H), 7.54 (d, 1H), 7.37 (d, 1H), 7.32 (d, 1H), 7.15-7.11 (m, 2H), 7.09 (bs, 1H), 4.94 (dd, 1H), 4.55-4.50 (m, 1H), 4.48-4.43 (m, 2H), 4.38 (q, 2H), 3.89-3.82 (m, 1H), 3.22 (bs, 4H), 3.00-2.96 (m, 2H), 2.90-2.84 (m, 1H), 2.62-2.57 (m, 2H), 2.56-2.49 (m, 4H), 2.18-2.16 (bs, 2H), 2.11-2.07 (m, 2H), 2.06-2.00 (m, 1H), 1.92-1.87 (m, 2H), 1.86-1.75 (m, 3H), 1.66-1.60 (m, 2H), 4.54-1.48 (m, 2H), 1.39 (t, 3H), 1.16-1.09 (m, 2H).

In addition, compound ARV-110 was purchased from BLD Pharmatech Ltd. (Cat. No.: BD01398519; Lot No.: CKA112, Purity: 97%) for the following assays. ARV-110 is a well-known bifunctional compound for AR degradation.

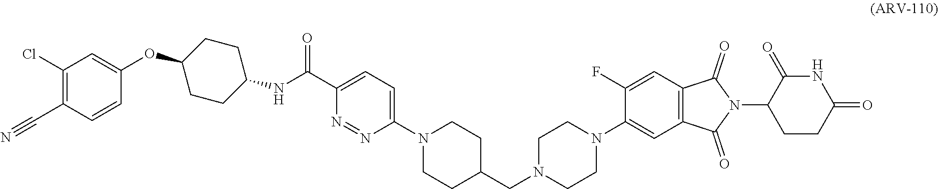 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

### Androgen Receptor Degradation Assay Using Western Blot Analysis

LNCaP.FGC cells (Cat. 60088, Bioresource Collection and Research Center, HsinChu City, Taiwan R.O.C.) grown in RPMI 1640 (Cat. 31800022, Thermo Fisher Scientific, Waltham, Massachusetts, United States) medium supplemented with 10% FBS (Cat. 10437028, Thermo Fisher Scientific, Waltham, Massachusetts, United States), 10 mM HEPES (Cat. 15630080, Thermo Fisher Scientific, Waltham, Massachusetts, United States) and 1 mM sodium pyruvate (Cat. 11360070, Thermo Fisher Scientific, Waltham, Massachusetts, United States) were seeded at 2×10⁵ cells per well in 24-well tissue culture plates. Cells were incubated at 37° C., 5% CO₂ for 24 hours (hr), then treated with 100 nanomolar concentrations (nM) any of the compounds 1 to 51 or ARV-110 for 24 hr. After the treatment, the cells were harvested, washed by PBS, and lysed with RIPA lysis and extraction buffer (Cat. 89900, Thermo Fisher Scientific, Waltham, Massachusetts, United States) supplemented with Halt Protease Inhibitor Cocktail (Cat. 78430, Thermo Fisher Scientific, Waltham, Massachusetts, United States) to collect protein samples.

The protein samples were separated by polyacrylamide gel electrophoresis, and then transferred to a piece of Immuno-Blot PVDF membrane (Cat. 1620177, Bio-Rad Laboratories, Hercules, California, United States). The presence of androgen receptor in the protein samples was detected by standard Western blotting procedure using an anti-AR antibody (1:2000 dilution) (Cat. 5153, Cell Signaling Technology Inc., Danvers, Massachusetts, United States) and a goat anti-rabbit HRP-conjugated secondary antibody (1:5000 dilution) (C04003, Croyez Bioscience Co., Ltd., Taipei City, Taiwan R.O.C.). The internal loading control GAPDH was detected using a mouse monoclonal antibody (1:5000) (GTX627408, GeneTex International Corp., HsinChu City, Taiwan R.O.C.) and a goat anti-mouse HRP-conjugated secondary antibody (1:5000 dilution) (C04001, Croyez Bioscience Co., Ltd., Taipei City, Taiwan R.O.C.). Chemiluminescence signals were developed using Clarity Western ECL substrate (Cat. 1705061, Bio-Rad Laboratories, Hercules, California, United States) and detected with digital imager iBright FL1500 (Invitrogen Corp., Carlsbad, California, United States).

Several compounds were selected and serial diluted (10×) with RPMI medium for treating LNCaP.FGC cells. A calibration curve was determined by the serial diluted samples for each compound, and the concentration needed for 50% AR degradation (AR DC₅₀) for each compound was calculated. The results are shown in Table 2.

In Table 2, the result marks “A” when AR DC₅₀ of the compound is less than 10 nM; the result marks “B” when AR DC₅₀ of the compound is equal to or greater than 10 nM and less than 25 nM; the result marks “C” when AR DC₅₀ of the compound is equal to or greater than 25 nM and less than 100 nM and the result marks “D” when AR DC₅₀ of the compound is equal to or greater than 100 nM. It is clear that most of the compounds of the present invention have good effect in AR degradation. Under the same operation conditions, many compounds of the present invention result in greater AR degradation than ARV-100 at the same concentration (100 nM), and they generally have a lower AR DC₅₀ (nM).

TABLE 2
AR DC50 (nM) of Compounds of the Present Invention

| | AR DC₅₀ |
|---|---|
| Cpd. No. | (nM) |
| 1 | A |
| 2 | A |
| 3 | A |
| 4 | C |
| 5 | C |
| 6 | C |
| 7 | C |
| 8 | C |
| 9 | C |
| 10 | A |
| 11 | A |
| 12 | B |
| 15 | C |
| 16 | D |
| 17 | B |
| 18 | B |
| 21 | A |
| 22 | B |
| 23 | B |
| 24 | C |
| 25 | B |
| 26 | D |
| 27 | B |
| 28 | C |
| 29 | B |
| 30 | A |
| 33 | A |
| 34 | B |
| 35 | B |
| 36 | B |
| 39 | B |
| 40 | B |
| 41 | D |
| 42 | C |
| 47 | C |
| 48 | B |
| 49 | B |
| 51 | A |
| ARV-110 | C |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1.-18. (canceled)

19. A bifunctional compound, or a pharmaceutically acceptable salt, hydrate, solvate, metabolite or prodrug thereof, wherein the bifunctional compound is represented by Formula (I): 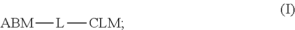
wherein:
ABM is an androgen receptor binding moiety;
-L- is a linking moiety; and
CLM is a cereblon E3 ubiquitin ligase binding moiety represented by Formula (II)-1: 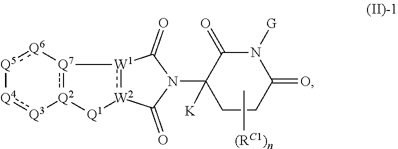
wherein: [symbol]
represents a single bond or a double bond;
one end of the -L- is covalently joined to Q₅, Q₄, Q₅ or Q₆; and the other end of the -L- is covalently joined to the ABM;
W¹ and W² are each independently CR^{C2} or N when a single bond is present between W¹ and W²; or, W¹ and W² are each C when a double bond is present between W¹ and W²;
G is selected from the group consisting of —H, —OH, —CH₂OH, —R^{C3}OC(═O)OR^{C4}, —R^{C3}OC(═O)NR^{C4}R^{C5}, and 2-(trimethylsilyl) ethoxymethyl group;
Q¹ is NR^{C6};
Q² and Q⁷ are each independently N or CR^{C2} when a single bond is present between Q² and Q⁷; or, Q² and Q⁷ are each C when a double bond is present between Q² and Q⁷;
when the one end of the -L- is covalently joined to any one atom selected from Q₃, Q₄, Q₅ and Q₆; the atom covalently joined with the -L- is CR^{C2} when two single bonds are each independently present between the C atom of CR^{C2} and its two adjacent atoms on the ring, or the atom covalently joined with the -L- is C when a double bond is present between the C atom and one of its two adjacent atoms on the ring; and, the other atoms selected from Q³, Q⁴, Q⁵ and Q⁶ which are not covalently joined with the -L- are each independently O, S, C(R^{C2})₂ or NR^{C2} when two single bonds are each independently present between the O atom, S atom, C atom of C(R^{C2})₂, or N atom of NR^{C2} and its two adjacent atoms on the ring, or the other atoms selected from Q³, Q⁴, Q⁵ and Q⁶ which are not covalently joined with the -L- are each Independently CR^{C2} when a double bond is present between the C atom of CR^{C2} and one of its two adjacent atoms on the ring;
K is selected from the group consisting of —H, an unsubstituted alkyl group, an alkyl group substituted by R^{C7}, an unsubstituted cycloalkyl group, and a cycloalkyl group substituted by R^{C7}; K is bound to the 6-membered ring with a stereospecific bond or a non-stereospecific bond;
R^{C1} is selected from the group consisting of an unsubstituted alkyl group, an alkyl group substituted by R^{C3}, an unsubstituted aryl group, an aryl group substituted by R^{C8}, an unsubstituted alkyl-aryl group, an alkyl-aryl group substituted by R^{C8}, an unsubstituted alkoxyl group, and an alkoxyl group substituted by R^{C8};
R^{C2} is selected from the group consisting of —H, -D, a halo group, —CH₂OH, —NR^{C4}R^{C5}, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, and an aryl group substituted by one or more halo groups;
R^{C3} is selected from the group consisting of an unsubstituted alkylene group, and an alkylene group substituted by R^{C7};
R^{C4} and R^{C5} are each independently selected from the group consisting of —H, an unsubstituted alkyl group, an alkyl group substituted by R^{C9}, an unsubstituted cycloalkyl group, a cycloalkyl group substituted by R^{C9}, an unsubstituted heterocyclyl group, a heterocyclyl group substituted by R^{C8}, an unsubstituted aryl group, an aryl group substituted by R^{C9}, an unsubstituted heteroaryl group, and a heteroaryl group substituted by R^{C9};
R^{C6} is selected from the group consisting of —CH₂OH, an unsubstituted C₁₋₆ alkyl group, and a C₁₋₆ alkyl group substituted by one or more halo groups;
R^{C7} is selected from the group consisting of a halo group, —CH₂OH, —NR^{C4}R^{C5}, 2-(trimethylsilyl) ethoxymethyl, an alkoxyl group, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C8} is selected from the group consisting of a halo group, —CH₂OH, —NR^{C4}R^{C5}, 2-(trimethylsilyl) ethoxymethyl, an unsubstituted cycloalkyl group, a cycloalkyl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C9} is selected from the group consisting of a halo group, —CH₂OH, 2-(trimethylsilyl) ethoxymethyl, and an alkoxyl group;
n is 0, 1, 2, 3 or 4; and
the -L- is represented by Formula (III): 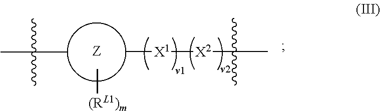
wherein
Z is selected from the group consisting of a 3- to 8-membered monocyclic ring, a 5- to 12-membered bicyclic ring, a 8- to 15-membered tricyclic ring and a 6- to 12-membered spiro bicyclic ring, each independently having 0 to 4 heteroatoms;
R^{L1} is selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, an unsubstituted C₁₋₆ alkoxyl group, a keto group, and an oxide group;
X¹ is a methylene group or an ethylene group, each of which is unsubstituted or substituted by alkyl or cycloalkyl;
X² is selected from the group consisting of a 5- to 8-membered arylene group, a 5- to 8-membered heteroarylene group having 1 to 3 heteroatoms, a 3- to 7-membered cyclic alkylene group, a 3- to 7-membered heterocyclic alkylene group having 1 to 2 heteroatoms, a 3- to 8-membered cyclic alkenylene group, a 3- to 8-membered cyclic heteroalkenylene group having 1 to 3 heteroatoms, and a 6- to 12-membered spiro bicyclic bivalent group having 0 to 4 heteroatoms, each of which is unsubstituted or substituted by alkyl or cycloalkyl;
m is 0, 1, 2, 3, 4, 5 or 6; v1 is 1 or 2; and v2 is 1 or 2; and
the heteroatom is selected from N, O and S.

20. The bifunctional compound, or the pharmaceutically acceptable salt, hydrate, solvate, metabolite or prodrug thereof as claimed in claim 19, wherein the CLM is represented by Formula (II)-2: 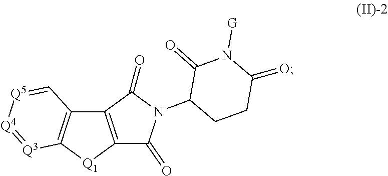
wherein
one end of the -L- is covalently joined to Q₅, Q₄ or Q₅;
G is selected from the group consisting of —H, —OH, and —CH₂OH;
Q¹ is NR^{C6}; when the one end of the -L- is covalently joined to any one atom selected from Q₃, Q₄ and Q₅; the atom attached with the -L- is C; and, the other atoms selected from Q³, Q⁴ and Q⁵ which are not attached with the -L- are each independently CR^{C2}; and
R^{C2} is selected from the group consisting of —H, -D, a halo group, an unsubstituted alkyl group, and an alkyl group substituted by one or more halo groups;
R^{C6} is selected from the group consisting of —CH₂OH, an unsubstituted C₁₋₆ alkyl group, a C₁₋₆ alkyl group substituted by one or more halo groups.

21. The bifunctional compound, or the pharmaceutically acceptable salt, hydrate, solvate, metabolite or prodrug thereof as claimed in claim 19, wherein the -L- is a linking moiety represented by Formula (III): 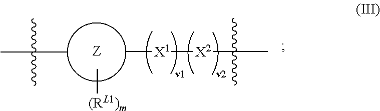
wherein
Z is selected from the group consisting of a 3- to 8-membered monocyclic ring, a 6- to 10-membered bicyclic ring and a 8- to 10-membered spiro bicyclic ring, each independently having 1 to 2 heteroatoms;
X¹ is an unsubstituted methylene group, or a methylene group substituted by alkyl or cycloalkyl;
X² is selected from the group consisting of a 3- to 7-membered heterocyclic alkylene group having 1 to 2 heteroatoms, a 3- to 8-membered cyclic heteroalkenylene group having 1 to 2 heteroatoms, and a 6- to 12-membered spiro bicyclic bivalent group having 1 to 2 heteroatoms, each of which is unsubstituted or substituted by alkyl or cycloalkyl;
m is 0, 1 or 2; v1 is 1 or 2; and v2 is 1.

22. The bifunctional compound, or the pharmaceutically acceptable salt, hydrate, solvate, metabolite, or prodrug thereof as claimed in claim 21, wherein the -L- is

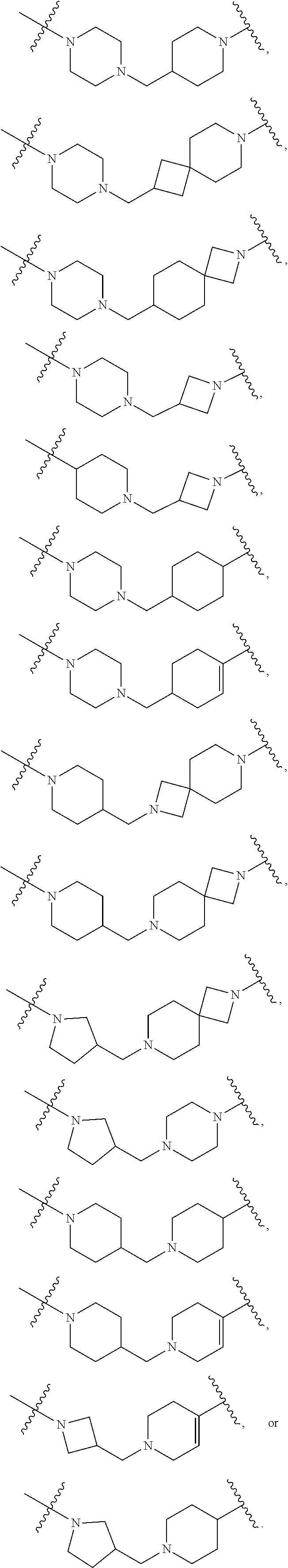

23. The bifunctional compound, or the pharmaceutically acceptable salt, hydrate, solvate, metabolite or prodrug thereof as claimed in claim 19, wherein the ABM is an androgen receptor binding moiety represented by 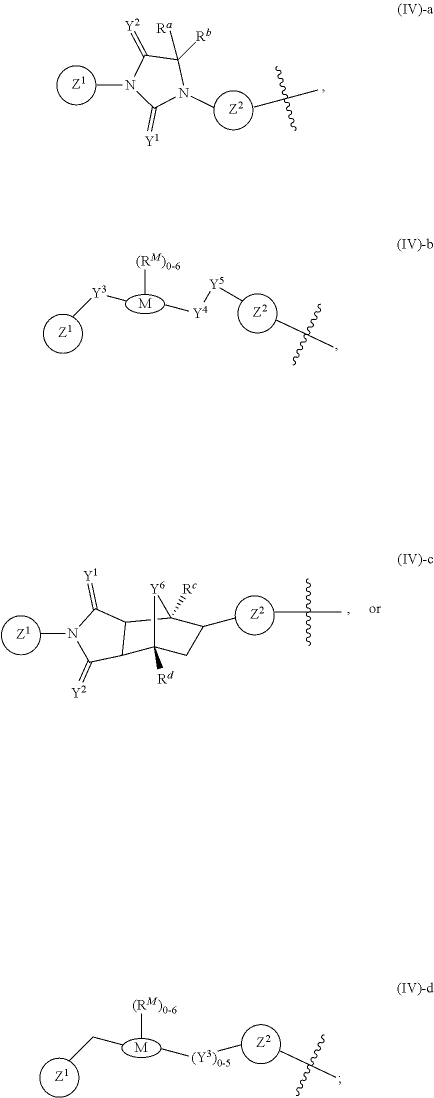
wherein:
Z¹ is selected from the group consisting of an aryl group, a heteroaryl group, a bicyclic group, or a bi-heterocyclic group, each independently substituted by one or more substituents independently selected from the group consisting of a halo group, a hydroxyl group, a nitro group, —CN, —C≡CH, an unsubstituted C₁₋₆ alkyl group, a C₁₋₆ alkyl group substituted by a C₁₋₆ alkoxyl group, a C₁₋₆ alkyl group substituted by one or more halo groups, an unsubstituted C₁₋₆ alkoxyl group, a C₁₋₆ alkoxyl group substituted by one or more halo groups, an unsubstituted C₂₋₆ alkenyl, a C₂₋₆ alkenyl substituted by one or more halo groups, an unsubstituted C₂₋₆ alkynyl, a C₂₋₆ alkynyl substituted by one or more halo groups, and any combinations thereof;
Y¹ and Y² are each independently NR^{Y1}, O or S;
Y³, Y⁴ and Y⁵ are each independently selected from the group consisting of a bond,
—O—, —NR^{Y2}—, —C(—R^{Y1})(—R^{Y2})—, —C(═O)—, —C(═S)—, —S(═O)—, —SO₂—, a heteroarylene group, and an arylene group;
Y⁶ is —N(—R^{Y1})—, —O— or —S—;
M is a 3- to 6-membered ring having 0 to 4 heteroatoms, which is unsubstituted or substituted by 0 to 6 R^{M} groups;
each R^{M} group is independently selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, and a C₁₋₆ alkoxyl group; or two R^{M} groups are taken together with the atom they attach to and form a 3- to 8-membered ring system containing 0 to 2 heteroatoms;
R^{a}, R^{b}, R^{c}, R^{d}, R^{Y1} and R^{Y2} are each independently selected from the group consisting of —H, an unsubstituted C14 alkyl group, a C₁₋₆ alkyl group substituted by a C₁₋₆ alkoxyl group, a Cie alkyl group substituted by one or more halo groups, a halo group, a C₁₋₆ alkoxyl group, a cyclic group, and a heterocyclic group; or R^{a}, R^{b} are taken together with the atom they attach to and form a 3- to 8-membered ring system containing 0 to 2 heteroatoms;
Z² is selected from the group consisting of a bond, a C₁₋₆ alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each or which is unsubstituted or substituted by 1 to 10 R^{Z2} groups;
each RZ group is independently selected from the group consisting of —H, a halo group, an unsubstituted C₁₋₆ alkyl group, a C₁₋₆ alkyl group substituted by one or more —F, —OR^{Z2A}, a C₃₋₆ cycloalkyl group, a C₄₋₆ cycloheteroalkyl group, an unsubstituted C₁₋₆ alkyl group, a C₁₋₆ alkyl group substituted by a C₁₋₃ alkyl group, a C₁₋₆ alkyl group substituted by a C₁₋₆ alkoxyl group, a C₁₋₆ alkyl group substituted by one or more halo groups, an unsubstituted heterocyclic group, a heterocyclic group substituted by a C₁₋₃ alkyl group, a heterocyclic group substituted by a C₁₋₆ alkoxyl group, a heterocyclic group substituted by one or more halo groups, an unsubstituted aryl group, an aryl group substituted by a Cis alkyl group, an aryl group substituted by a C₁₋₆ alkoxyl group, an aryl group substituted by one or more halo groups, an unsubstituted heteroaryl group, a heteroaryl group substituted by a Cis alkyl group, a heteroaryl group substituted by a C₁₋₆ alkoxyl group, a heteroaryl group substituted by one or more halo groups, a bicyclic heteroaryl group, an unsubstituted Cis alkoxyl group, and a C₁₋₃ alkoxyl substituted by one or more groups selected from —F, —OH, —NH₂, —NR^{Y1}R^{Y2} and —CN; and
R^{Z2A} is selected from the group consisting of H, a C₁₋₆ alkyl group, and a C₁₋₆ heteroalkyl group, each of which is unsubstituted or substituted by a cycloalkyl group, a cycloheteroalkyl group, an aryl group, a heterocyclic group, a heteroaryl group, a halo group, or a C₁₋₃ alkoxyl group.

24. The bifunctional compound, or the pharmaceutically acceptable salt, hydrate, solvate, metabolite or prodrug thereof as claimed in claim 23, wherein Z¹ is selected from the group consisting of

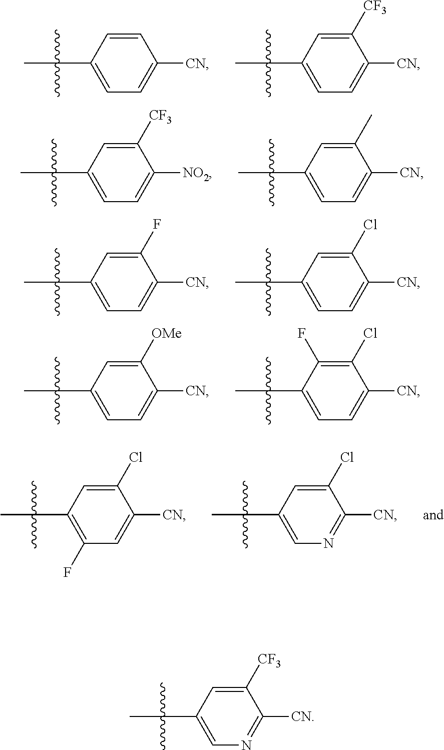

25. The bifunctional compound, or the pharmaceutically acceptable salt, hydrate, solvate, metabolite or prodrug thereof as claimed in claim 23, wherein Z² is selected from the group consisting of

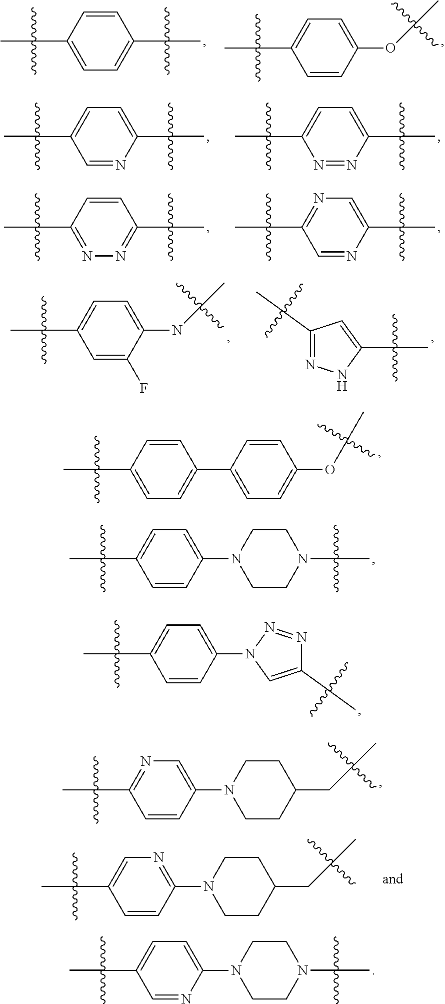

26. The bifunctional compound, or the pharmaceutically acceptable salt, hydrate, solvate, metabolite or prodrug thereof as claimed in claim 23, wherein the ABM is selected from the group consisting of 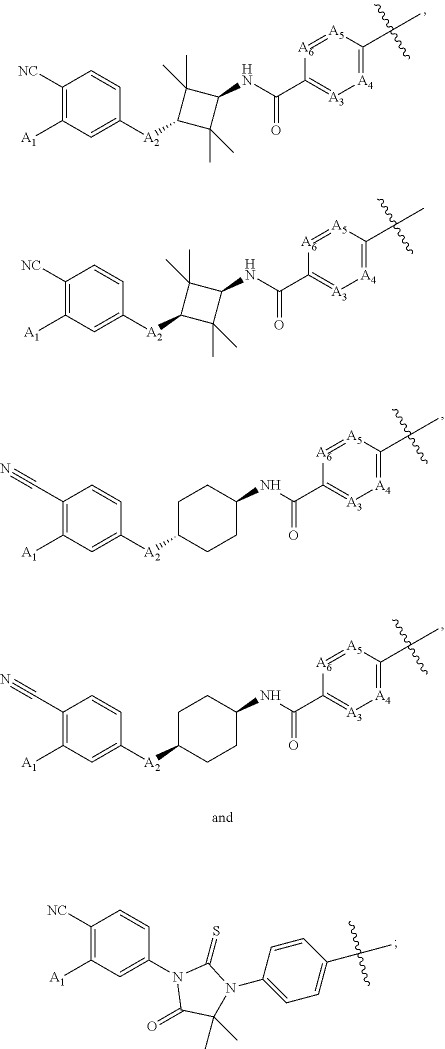
wherein:
A₁ is selected from —C1, —F, —Br or —CF₃;
A₂ is selected from —O—, —NH—, —N(-methyl)- or —N(-ethyl); and
A₃, A₄, A₅ and A₆ are each independently —CH— or —N—.

27. The bifunctional compound, or the pharmaceutically acceptable salt, hydrate, solvate, metabolite or prodrug thereof as claimed in claim 19, wherein the bifunctional compound is selected from the group consisting of:

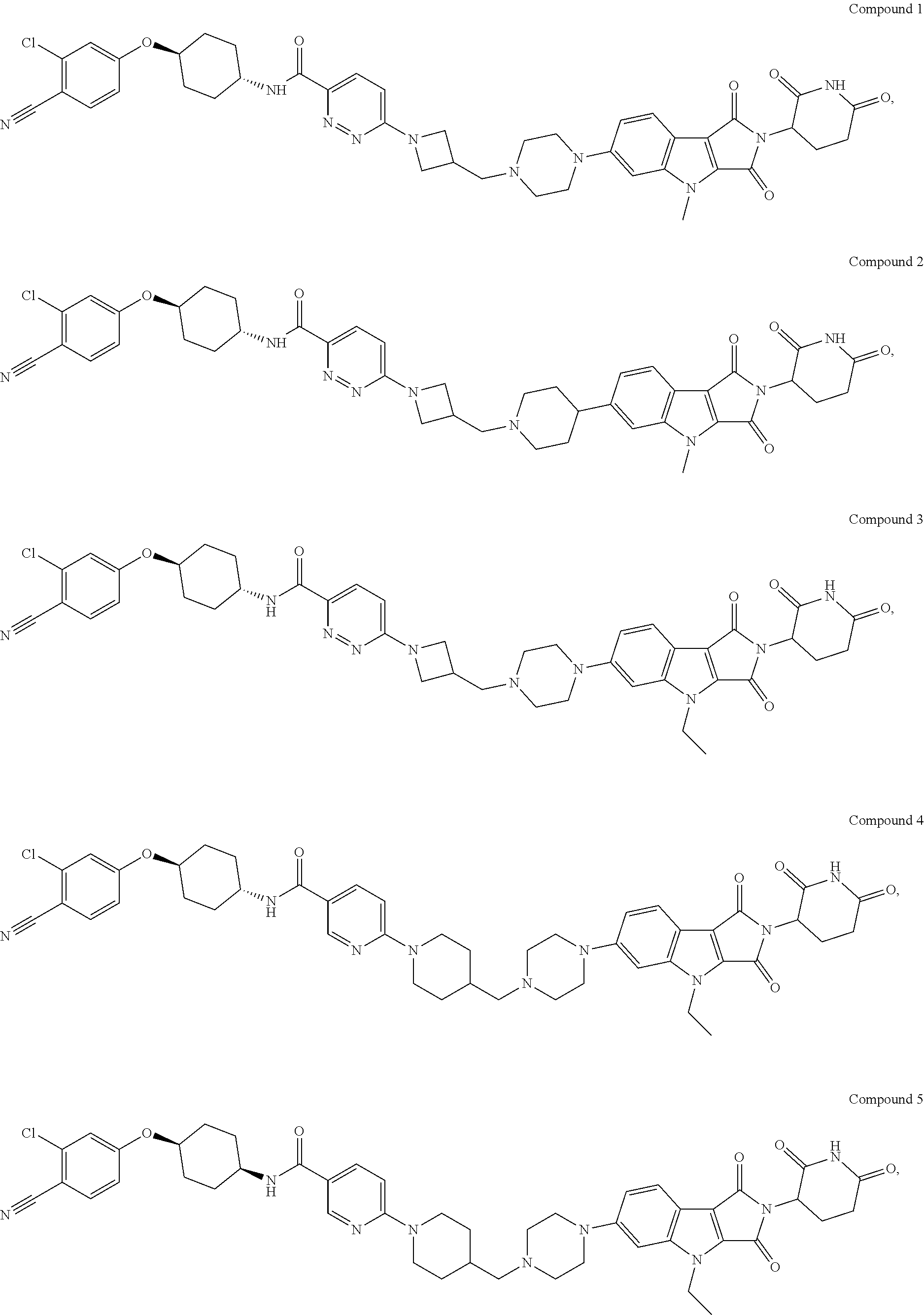

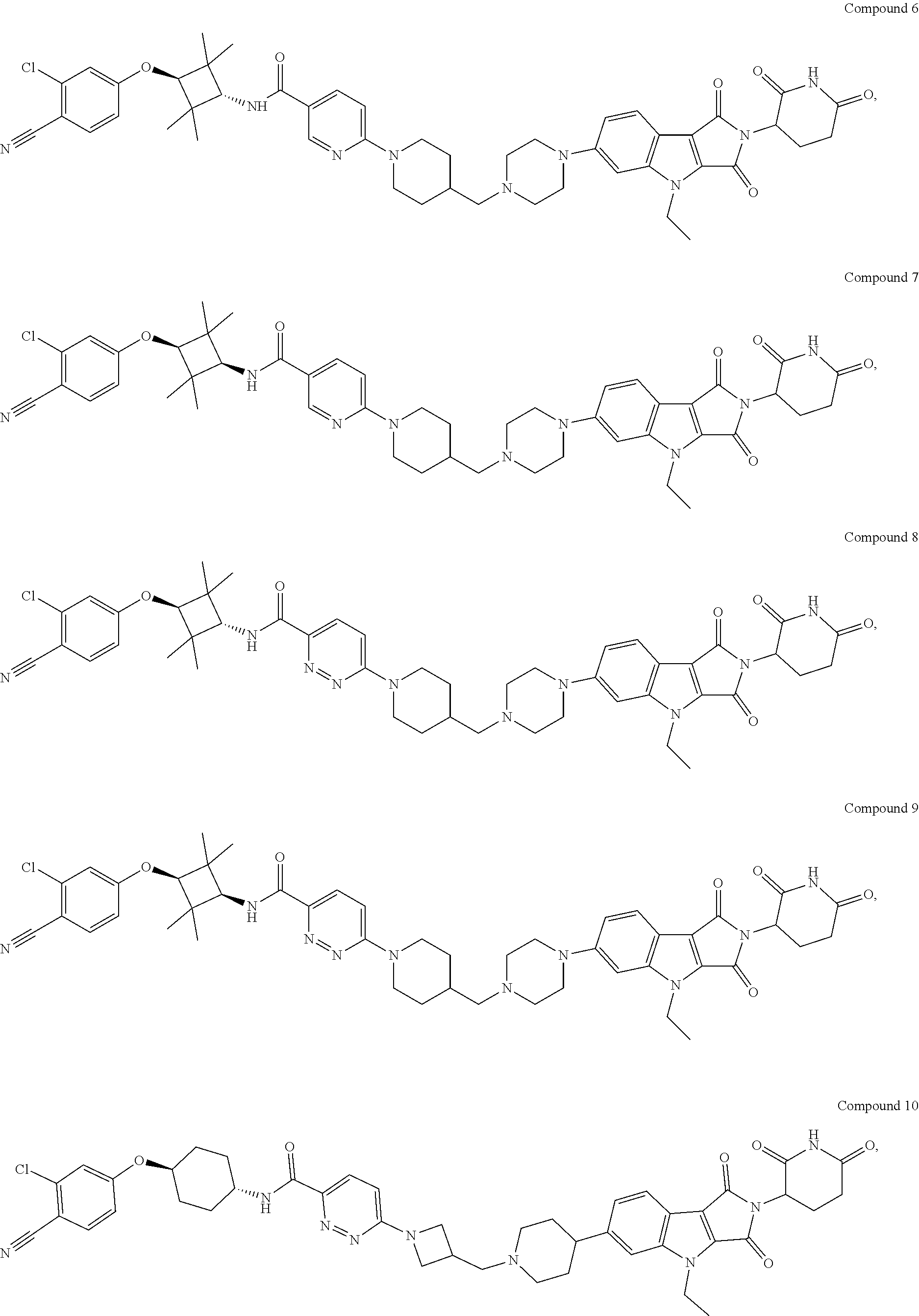

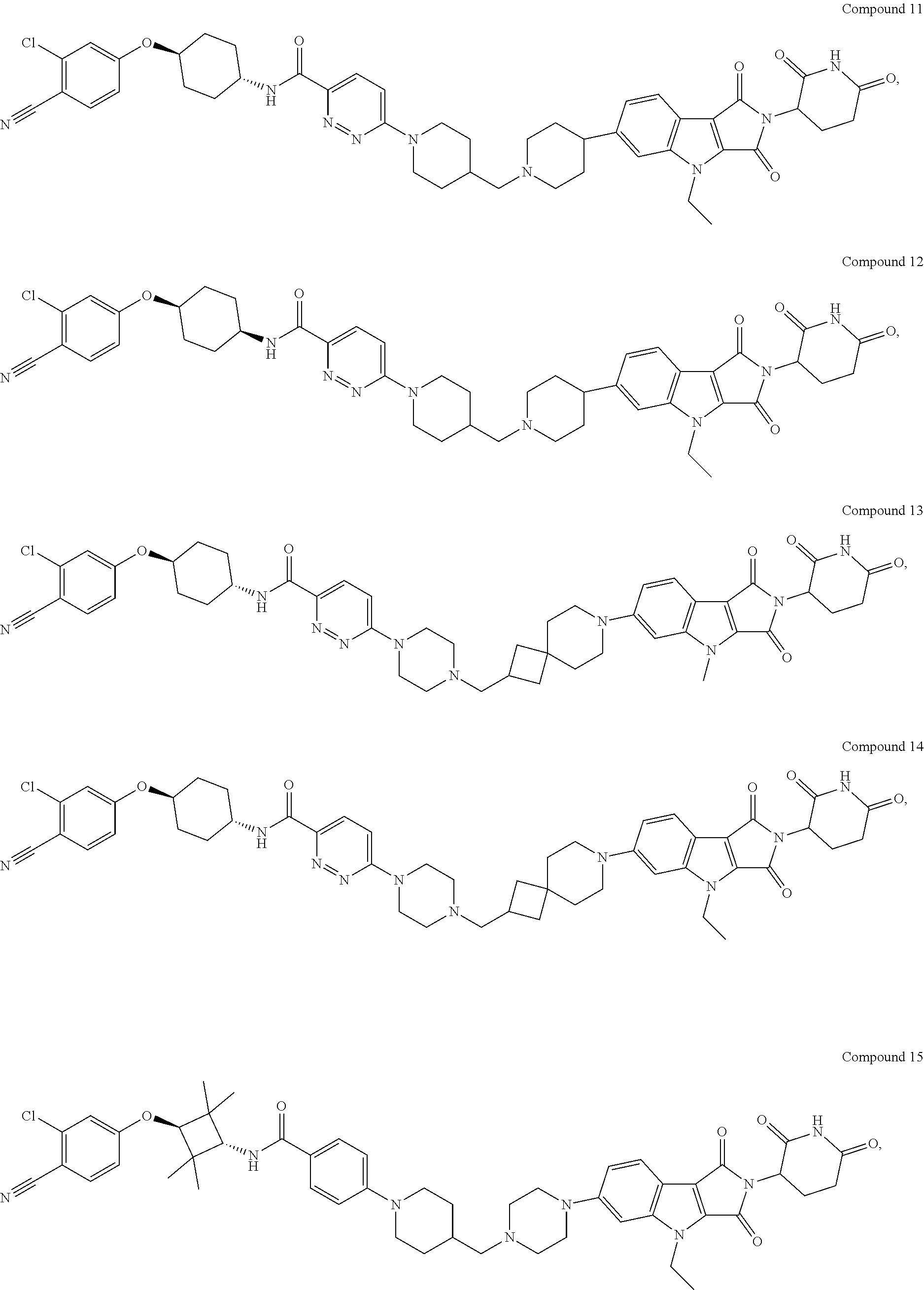

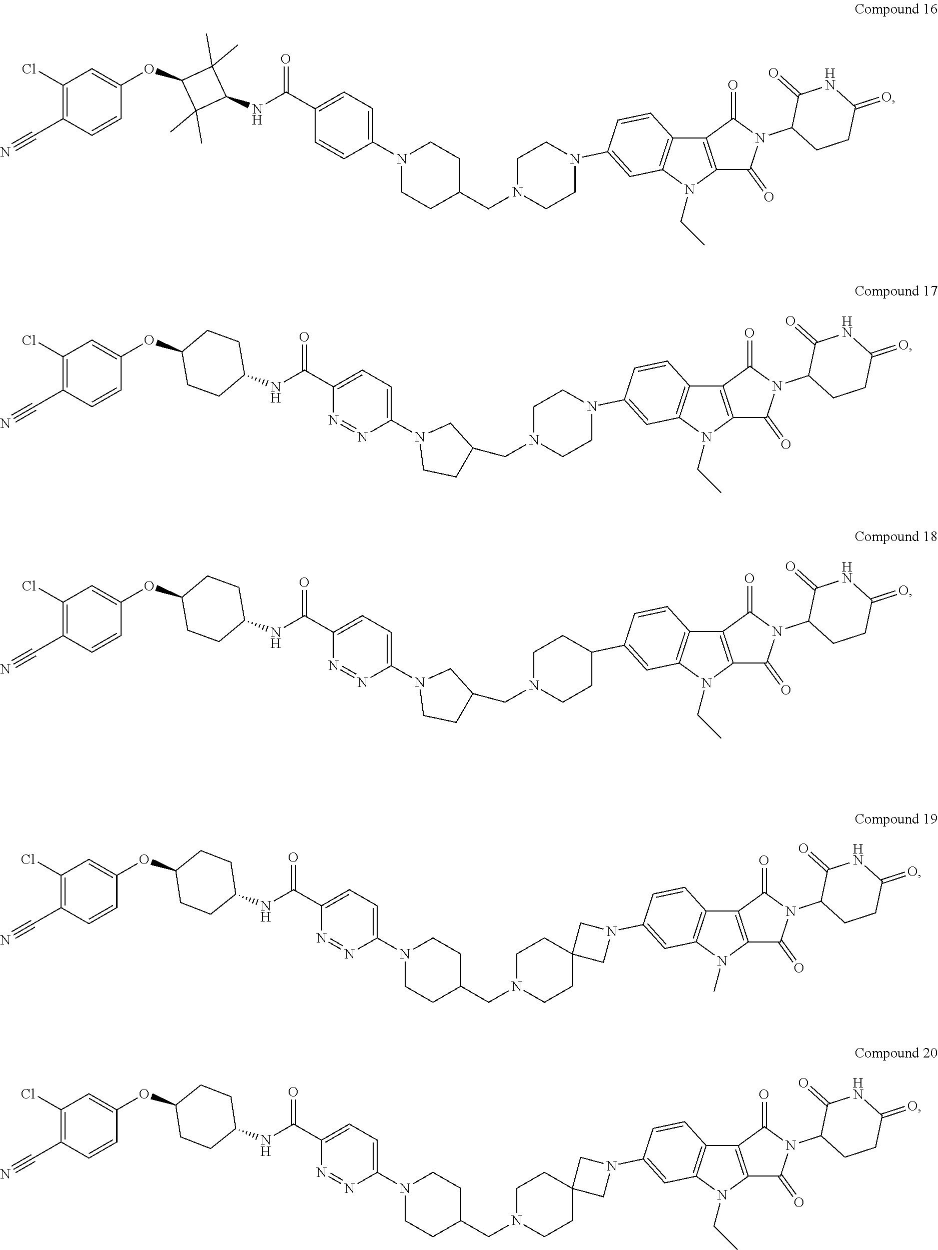

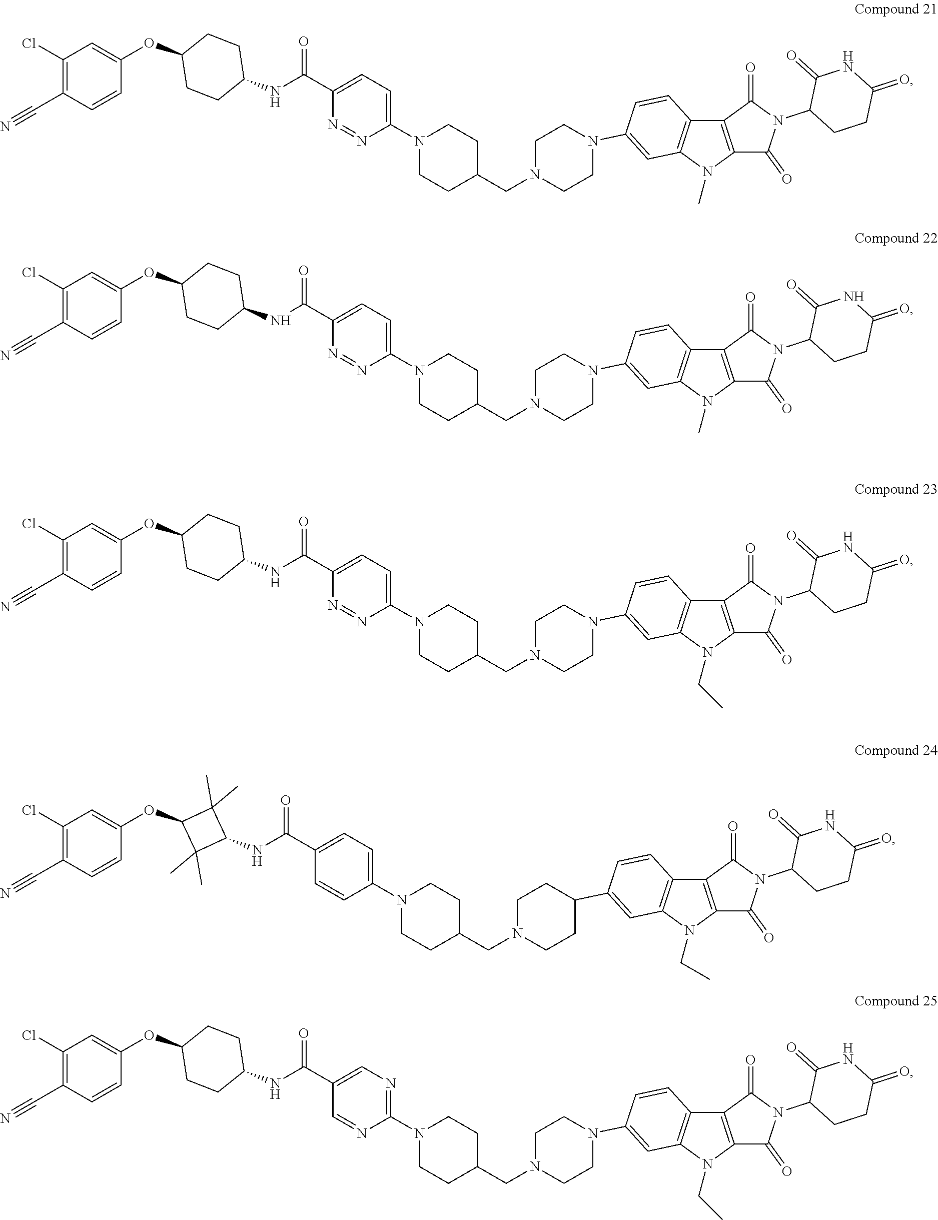

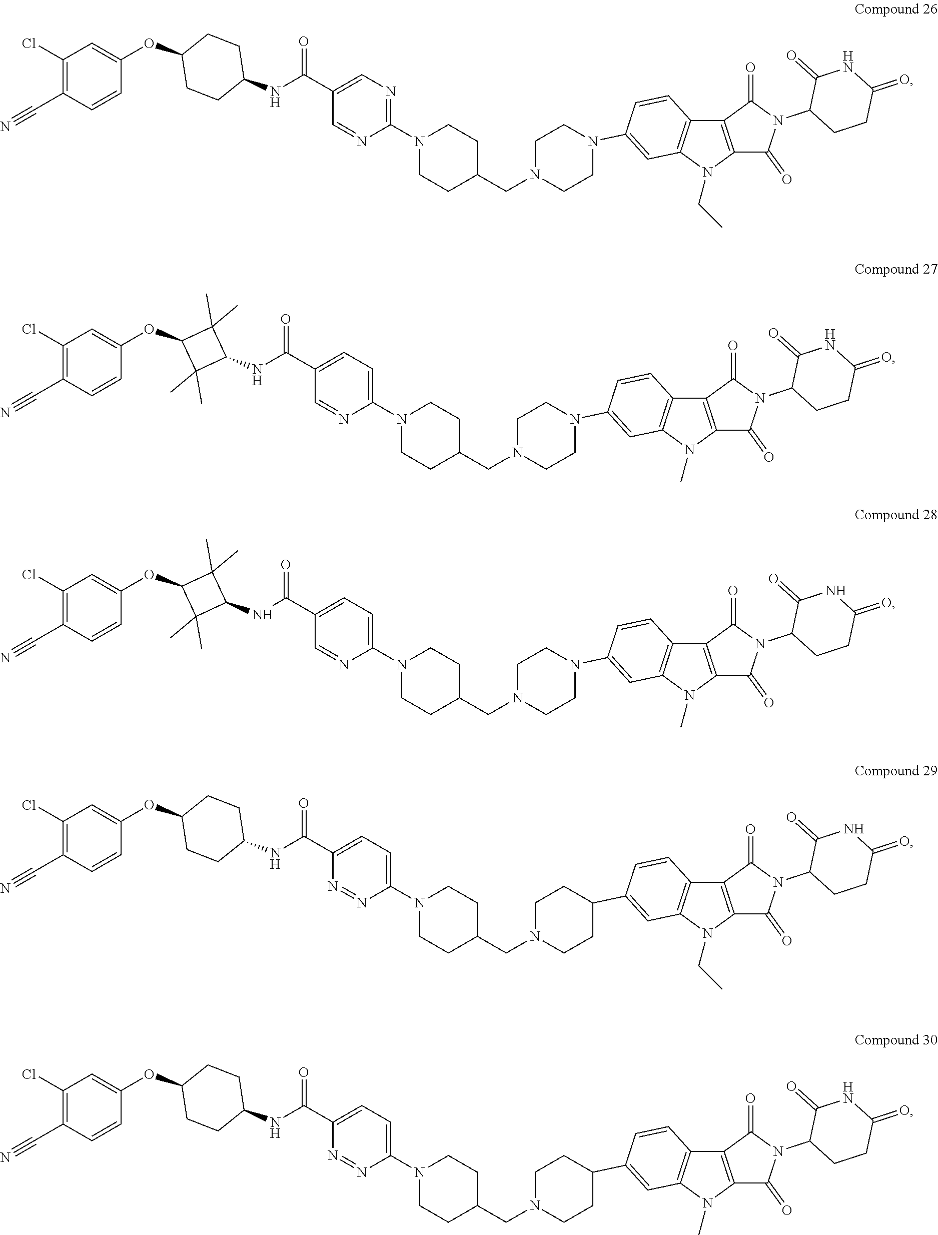

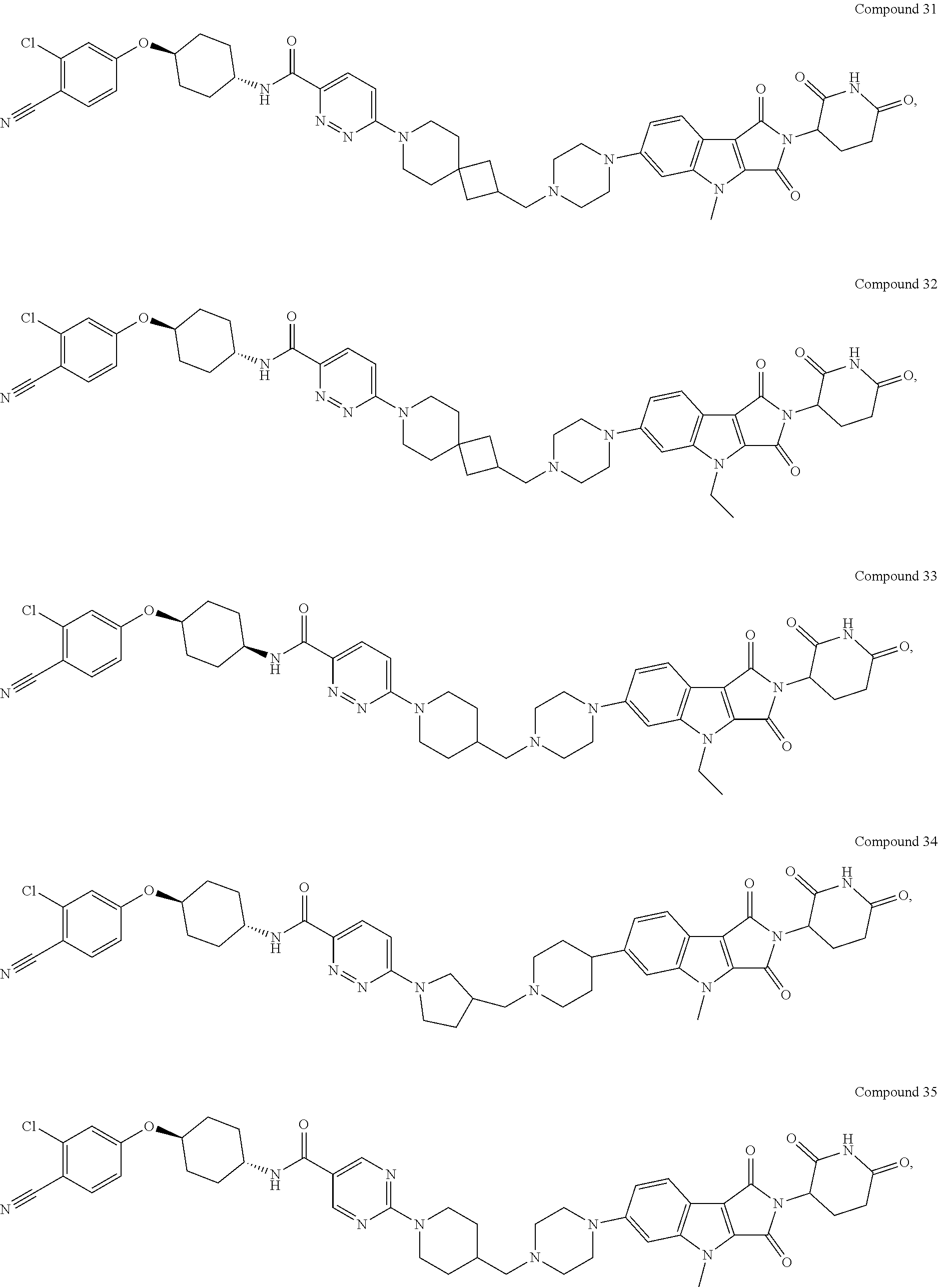

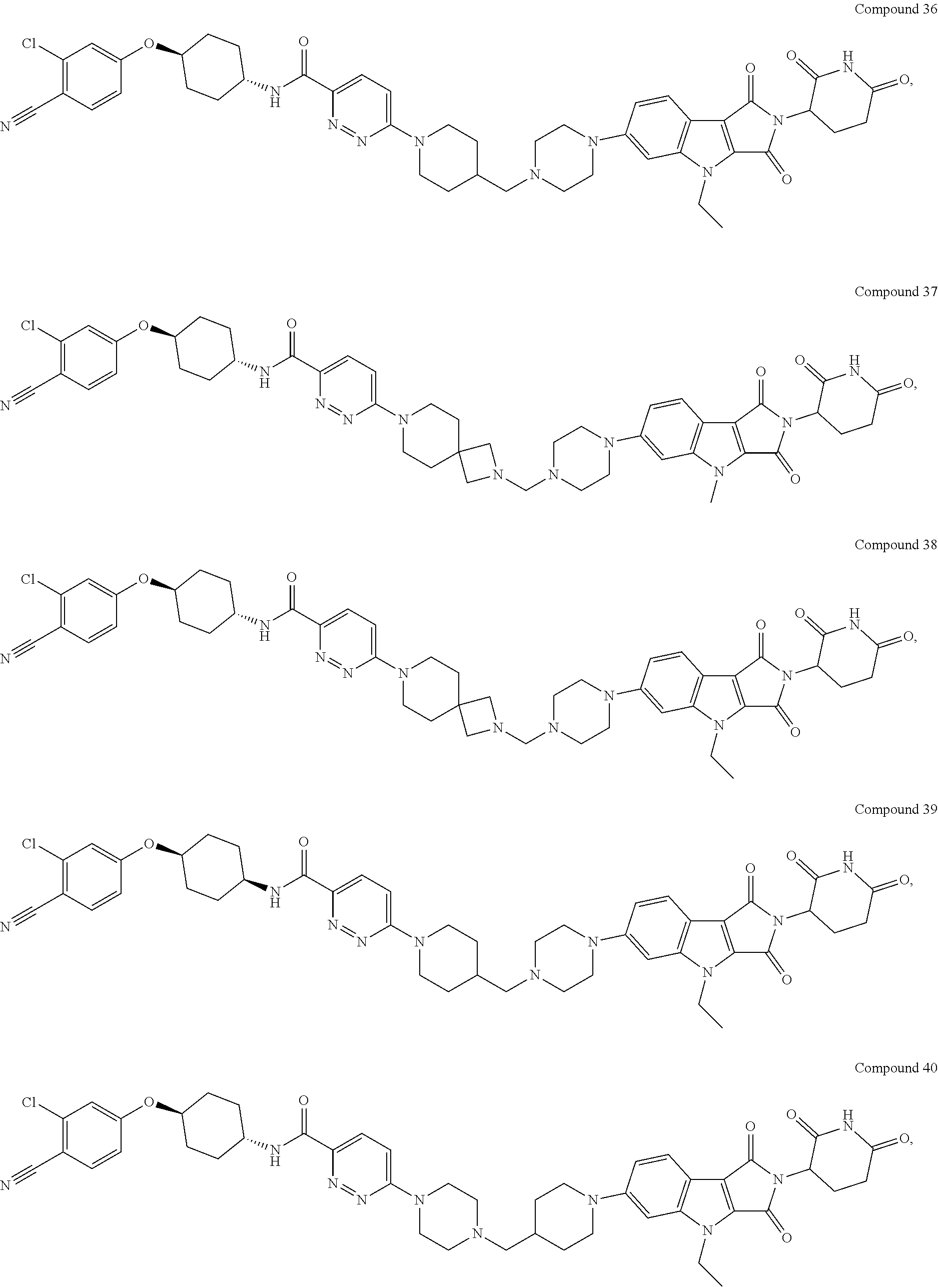

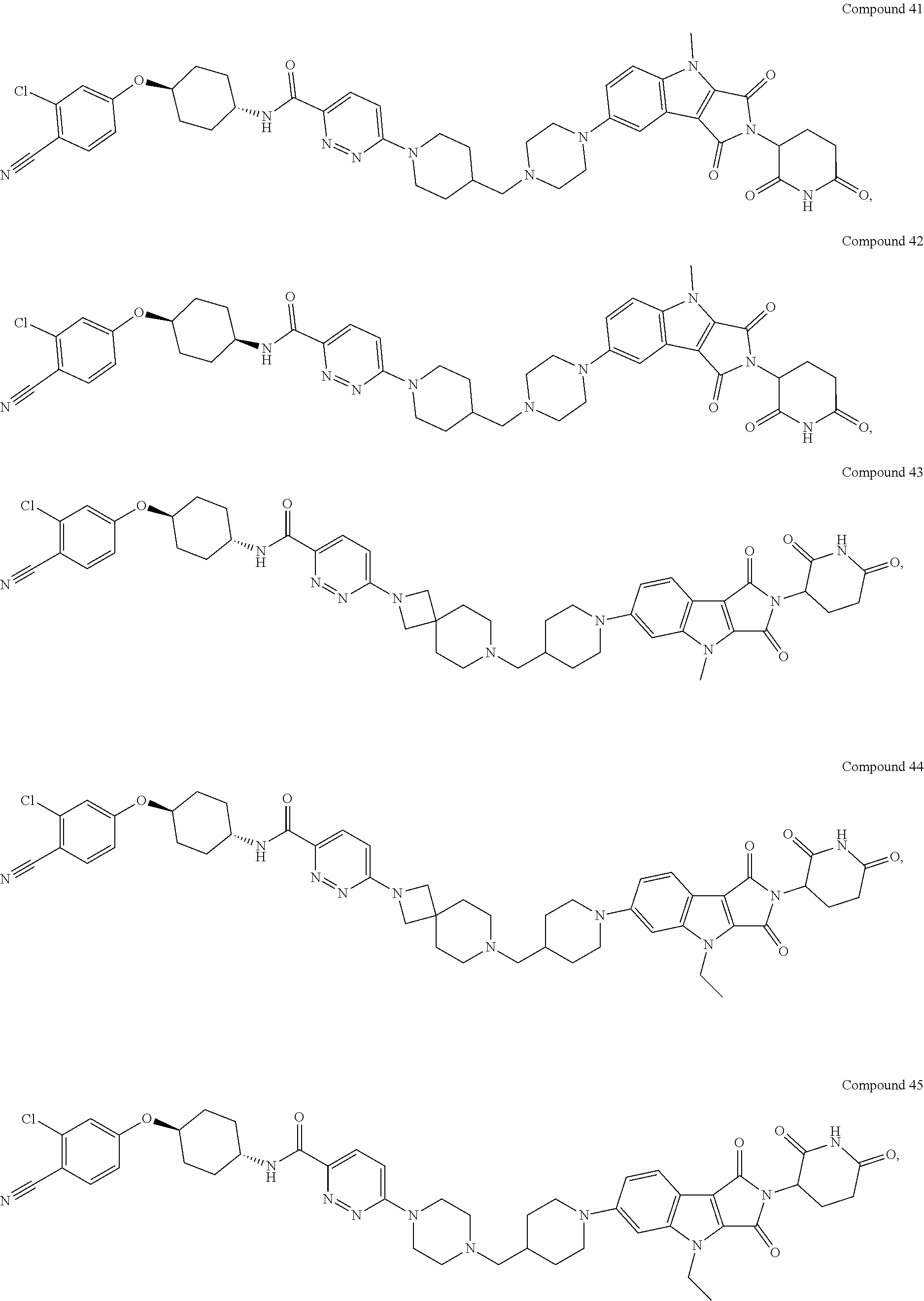

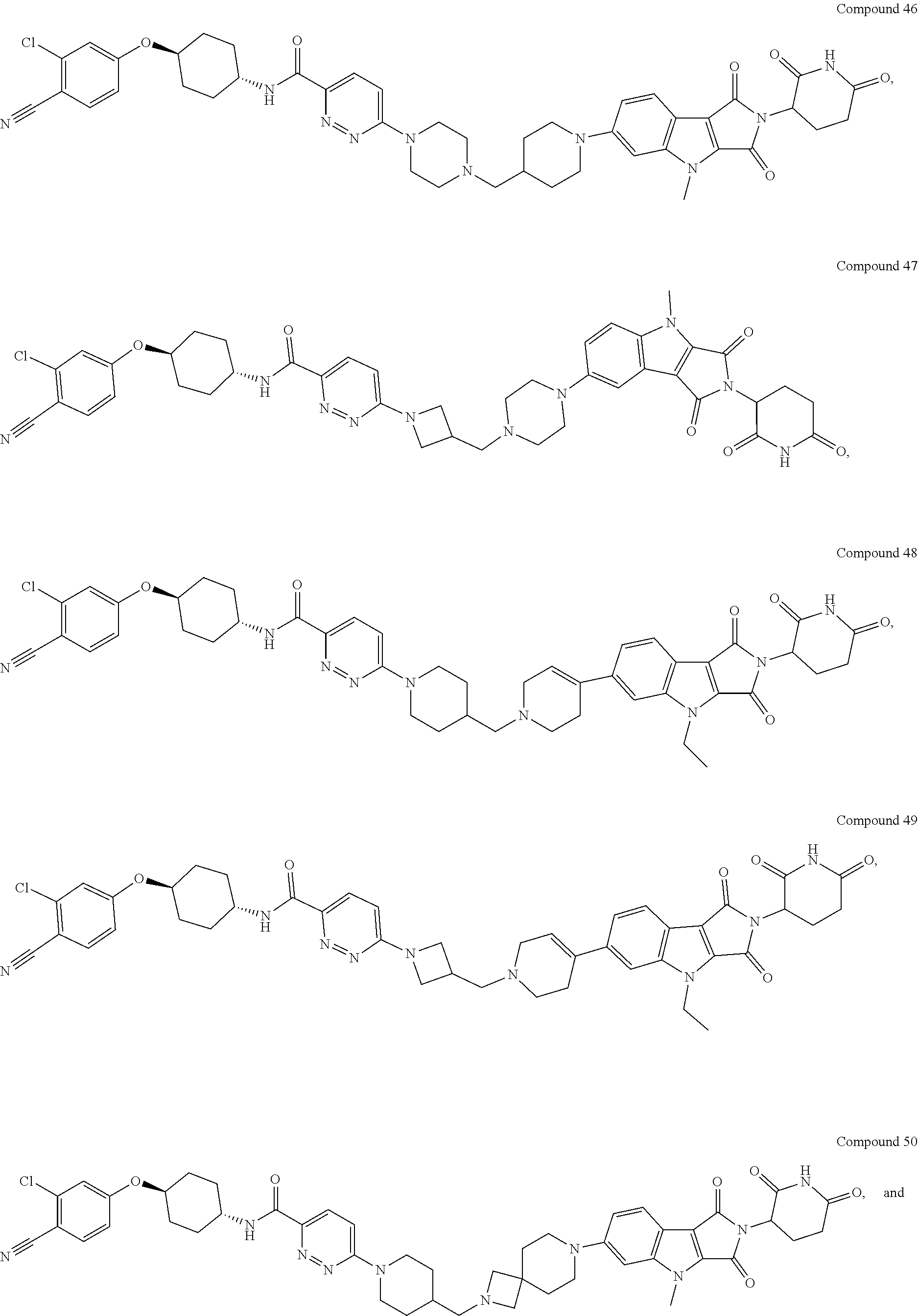

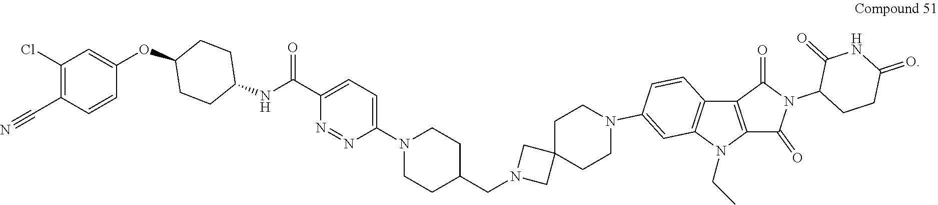 [SMILES: CCn1c2c(c3ccc(N4CCC5(CC4)CN(CC4CCN(c6ccc(C(=O)N[C@H]7CC[C@H](Oc8ccc(C#N)c(Cl)c8)CC7)nn6)CC4)C5)cc31)C(=O)N(C1CCC(=O)NC1=O)C2=O]

28. A pharmaceutical composition comprising an effective amount of the compound, or the pharmaceutically acceptable salt, hydrate, solvate, metabolite or prodrug thereof as claimed in claim 19; and a pharmaceutically acceptable carrier.

29. The pharmaceutical composition of claim 28, further comprising a second therapeutic agent.

30. A method for treating an androgen receptor related disease in a subject in need thereof, comprising administering an effective amount of the compound, or the pharmaceutically acceptable salt, hydrate, solvate, metabolite or prodrug thereof as claimed in claim 19 to the subject.

31. A method for treating an androgen receptor related disease in a subject in need thereof, comprising administering an effective amount of the pharmaceutical composition as claimed in claim 28 to the subject.

32. The method of claim 30, wherein the androgen receptor related disease is an androgen receptor related cancer or an androgen receptor related skin disease.

33. The method of claim 32, wherein the androgen receptor related cancer is breast cancer or prostate cancer.

34. The method of claim 33, wherein the prostate cancer is castration-resistant prostate cancer.

35. The method of claim 33, wherein the breast cancer is triple negative breast cancer.

36. The method of claim 32, wherein the androgen receptor related skin disease is androgenetic alopecia, acne, hidradenitis suppurativa, hirsutism, atopic dermatitis.

37. The method of claim 30, further comprising administering an effective amount of a second therapeutic agent.
